(19) United States
(12) Patent Application Publication
(10) Pub. No.: US 2019/0038524 A1
(43) Pub. Date: Feb. 7, 2019

(54) HAIR COSMETIC MATERIAL COMPOSITION AND OXIDIZING AGENT-CONTAINING COMPOSITION THEREOF, HAIR COSMETIC MATERIAL, AND HAIR COSMETIC PRODUCT

(71) Applicant: HOYU CO., LTD., Nagoya-shi (JP)

(72) Inventors: Yoshihiro KONNO, Nagakute-shi (JP); Shinya KANAYAMA, Nagakute-shi (JP); Takeshi KOMODA, Nagakute-shi (JP)

(73) Assignee: HOYU CO., LTD.

(21) Appl. No.: 16/156,524

(22) Filed: Oct. 10, 2018

Related U.S. Application Data
(62) Division of application No. 14/898,626, filed on Dec. 15, 2015.

(30) Foreign Application Priority Data
Jun. 28, 2013 (JP) 2013-136475
Jun. 28, 2013 (JP) 2013-136476
Jul. 2, 2013 (JP) 2013-139321
Jul. 5, 2013 (JP) 2013-142176

(51) Int. Cl. A61K 8/04; B65D 83/14; A61Q 5/08; A61Q 5/10; B65D 83/62; B65D 83/68; A45D 19/02

(52) U.S. Cl. CPC A61K 8/046; B65D 83/752; A61Q 5/08; A61Q 5/10; B65D 83/62; B65D 83/682; A45D 2019/025; A61K 2800/882; A45D 2200/058; A61K 2800/4324; A61K 2800/87; B65D 83/68

(57) ABSTRACT

A hair cosmetic material includes a first agent containing an alkali agent and a second agent containing an oxidizing agent. These first agent and second agent are used in a double structure container provided with a mechanism of separating the first agent and the second agent from each other and simultaneously discharging the both agents by a propellant. In the double structure container, a space for filling the first agent and a space for filing the second agent are each independently provided in the inside of a compressed gas filling space having a propellant for pressurization filled therein. Each of the first agent and the second agent is discharged in a liquid state, and each of the first agent and the second agent has a viscosity falling within the range of from 7,000 to 30,000 mPa·s at 25° C.

### CROSS-REFERENCE TO RELATED APPLICATIONS

This is a divisional application of Ser. No. 14/898,626 filed Dec. 15, 2015. It is to be noted that the present application claims priorities based on Japanese Patent Application No. 2013-136475 filed in Japan Patent Office on Jun. 28, 2013, Japanese Patent Application No. 2013-136476 filed in Japan Patent Office on Jun. 28, 2013, Japanese Patent Application No. 2013-139321 filed in Japan Patent Office on Jul. 2, 2013, and Japanese Patent Application No. 2013-142176 filed in Japan Patent Office on Jul. 5, 2013, and the entireties of Japanese Patent Application No. 2013-136475, Japanese Patent Application No. 2013-136476, Japanese Patent Application No. 2013-139321, and Japanese Patent Application No. 2013-142176 are incorporated in the present international application by reference.

### TECHNICAL FIELD

The invention disclosed in the present application relates to a hair cosmetic material composition. In detail, the present invention relates to an aerosol-type hair cosmetic material composition to be used upon being filled in an aerosol container that is a double structure container.

In addition, the invention disclosed in the present application relates to an oxidizing agent-containing composition of a hair cosmetic material composition. In detail, the invention relates to an oxidizing agent-containing composition that is a second agent to be used in a double structure container in which a first inner container for which a first agent that is an alkali-containing composition is filled and a second inner container for which the second agent that is an oxidizing agent-containing composition is filled are accommodated in the same outer container.

Furthermore, the invention as disclosed in the present application relates to a hair cosmetic material and a hair cosmetic material product. In detail, the invention relates to a hair cosmetic material containing a first agent and a second agent adapted especially for a double structure container provided with a specified mechanism for discharging the filled first agent and second agent; and a hair cosmetic material product which is constituted to include this hair cosmetic material and a double structure container, and in which the first agent and the second agent of the hair cosmetic material are filled in the double structure container.

### BACKGROUND ART

A first agent containing an alkali agent and a second agent containing an oxidizing agent are mixed at the time of use. In a so-called aerosol-type hair cosmetic material product utilizing a propellant, a first agent and a second agent of a hair cosmetic material composition are separately filled in an aerosol container and discharged from the aerosol container at the time of use.

The aerosol container in which the first agent, the second agent, and the propellant are filled in separate spaces is a double structure container. In the double structure container, the propellant provides a pressure for discharging the first agent filled in a first inner container and the second agent filled in a second inner container, and the inner containers cause elastic deformation due to the pressure, whereby a filled material becomes possible to be discharged in a cream state as it is. A compressed gas and a liquefied gas can be used as the propellant.

The following PTL 1 discloses a so-called “duplex can” type aerosol container (see PTL 1). In one specific example of this aerosol container, a propellant that is the compressed gas and a first agent or a second agent are filled in a first container and a second container, respectively, and it is possible to discharge each of the first agent and the second agent in a cream state as it is. As is clear from the structure of this aerosol container, the first container and the second container may be different from each other in terms of a propellant amount/pressure in the container. Since the propellant amount/pressure in the container can be freely set in each container, such an aerosol container is liable to cope with the case where the first agent and the second agent have different viscosity/viscousness from each other, or oxygen generated from an oxidizing agent (especially hydrogen peroxide) as the second agent.

The following PTL 2 discloses an aerosol container which can be used in a hair cosmetic material composition. The following PTL 2 discloses a double structure container in which a first inner container and a second inner container are existent in the same outer container.

The following PTL 3 discloses a double structure aerosol container in which a first inner container for filling a first undiluted solution and a second inner container for filling a second undiluted solution are accommodated in the same outer container.

The following PTL 4 discloses a second agent that is said to be excellent in mixing properties with a first agent, not sticky at the time of application to the hair, excellent in extensibility, good in compatibility with a hair, excellent in a bleaching power, uniform in an emulsified state at the time of preparation, and appropriate in viscosity.

There is proposed a double structure container provided with such a mechanism that not only a first agent and a second agent of a hair cosmetic material are separately filled, but also the both agents are simultaneously discharged from the same pressure discharge system (hereinafter referred to as “separate filling/same pressure discharge-type double structure container”). As the separate filling/same pressure discharge-type double structure container, for example, those having the following mechanisms can be exemplified.

That is, an opening of the outer container in which the propellant that is a compressed gas or liquefied gas for pressurization is filled therein (propellant filling space) is airtightly closed by a lid provided with a discharge passage and a valve for opening and closing this. In addition, two bag-like bodies that are inner containers in which the first agent and the second agent of the hair cosmetic material are filled, respectively (a space for filling the first agent and a space for filling the second agent) are placed in the inside of the outer container, and openings of these bag-like bodies are communicated in a liquid-tight manner with the discharge passage of the lid. In consequence, the first agent and the second agent filled in the bag-like bodies always receive a discharge pressure by the same propellant, and the simultaneous discharge of the first agent and the second agent can be controlled by a simple opening and closing operation of a discharge passage valve.

For example, the following PTLs 3 and 5 disclose a double structure container basically provided with such a mechanism.

### CITATION LIST

### Patent Literature

PTL 1: JP-A-2002-240873

PTL 2: JP-A-2001-122364

PTL 3: JP-A-2012-229318

PTL 4: JP-A-2007-217293

PTL 5: JP-A-2013-043659

PTL 6: JP-A-2010-235578

PTL 7: JP-A-2007-314442

### SUMMARY OF INVENTION

### Technical Problem

In the double structure container disclosed in the foregoing PTL 2, since the first inner container and the second inner container are existent in the same outer container, the first agent and the second agent are placed under the same pressure. Therefore, the first agent and the second agent cannot be placed under a different pressure from each other as in the duplex can-type aerosol container. Moreover, there is involved such a problem that after storing the aerosol-type hair cosmetic material product for a certain period of time, it is desired to discharge the first agent and the second agent in a desired ratio.

However, the double structure container has such advantages that the number of constituent components is small; and that it can be produced at low costs. In addition, the double structure container has such an advantage that a degree of freedom for design with respect to the shape of the outer container is high.

In the duplex can-type aerosol container, two double structure containers stand in line and are formed in a wide shape, and hence, from the viewpoint of easiness in grasping the aerosol container, there was room for improvement. The double structure container can be made easy for grasping by forming the outer container in an approximately columnar shape.

Taking the foregoing into consideration, the inventors of the present application determined to adopt a double structure container.

Commercially available duplex can-type inner containers are formed in a cylindrical shape. When the discharge of filled materials is continued, the right and left of the inner container cramp up, and the inner container is not crushed tightly. Therefore, though flow passages of the filled materials in the inner container are ensured, residual amounts of the filled materials which cannot be discharged become large.

The inventors of the present application thought that in a double structure container, if a pouch container formed by sticking elastic deformable sheet materials together is used as an inner container, when the discharge is continued, the sheet materials are associated together and crushed tightly, whereby the residual amounts of the filled materials can be lessened. However, at the stage where large amounts of the filled materials remain, the flow passages of the filled materials are plugged, and the movement of the filled agents becomes worse. As a result, the residual amounts in the pouch container could not be lessened.

The inventors of the present application made extensive and intensive investigations. As a result, it has been found that when an innermost layer of a pouch container is formed of a polyolefin resin layer, and a content ratio of a nonionic surfactant and a higher alcohol of an agent to be filled in the pouch container is allowed to fall within a fixed range, the residual amount of the filled material can be lessened.

One aspect of the present invention is to provide an aerosol-type hair cosmetic material composition capable of lessening the residual amount in a pouch container.

There is often the case where the first agent of the aerosol-type hair cosmetic material composition contains larger amounts of an oily component and a surfactant as compared with those in the second agent. Therefore, there is a tendency that the residual amount of the first agent in the inner container is larger than that of the second agent. The invention as disclosed in the present application is useful in view of the fact that the residual amount of the first agent can be lessened.

In commercially available aerosol-type hair cosmetic material products, an outer container is constituted of a light-impermeable material, such as aluminum, etc., and the residual amount cannot be visually recognized. Thus, the residual amount could not be precisely judged. For the purpose of preventing a situation that the hair cosmetic material composition is short on the way of a treatment operation, some persons coped with this situation by making up a plurality of the same products ready to hand.

Then, if the whole or a part of the outer container in the aerosol container is made light-permeable, the shape of the inner container having the first agent or second agent filled therein can be visually recognized. In addition, in the case where the inner container itself is light-permeable, the residual amount of the filled material can be visually recognized. Therefore, in the outer container and the inner container of the aerosol container, if the light-permeable material is used, the defect that in the aerosol-type hair cosmetic material product which has hitherto been made commercially available, the residual amount could not be visually recognized can be dissolved.

However, while hydrogen peroxide that is an oxidizing agent is excellent in a bleaching power of melamine in the hair, there is room for improvement in storage stability. In particular, the hydrogen peroxide becomes instable under irradiation with sunlight.

There is also involved such a defect that when the hydrogen peroxide is decomposed, not only its oxidizing power is lowered, but also generated oxygen moves into the propellant filling space. In addition, when the oxygen generated by decomposition of hydrogen peroxide resides in the second inner container, on the occasion of discharging the second agent from the double structure container, there is a possibility that the oxygen is released under atmospheric pressure simultaneously with the second agent.

In the foregoing PTL 4, the uniformity of emulsion and the appropriateness of viscosity at the time of preparation of the second agent are evaluated. However, in the foregoing PTL 4, the storage of the second agent under irradiation with sunlight is not discussed at all.

In the double structure container in which the first inner container and the second inner container are existent in the same outer container, if the outer container and the second inner container are constituted to include a light-permeable material, thereby making the residual amount of the second agent in the second container visible, the case where the second agent is placed under irradiation with sunlight is assumed, and an enhancement of the stability of hydrogen peroxide becomes important.

The matter that the hair cosmetic material product utilizing the aerosol container can be set aside in plural times and used is one of advantages. The hair cosmetic material product in an unopened state is frequently encased (in a light-shielded state), stored, and circulated. When the hair cosmetic material product is once opened, the aerosol container is frequently stored in the state exposed to sunlight.

Then, in order to enhance the stability of hydrogen peroxide in the second agent under irradiation with sunlight, the inventors of the present application made extensive and intensive investigations. As a result, it has been found that the stability of the hydrogen peroxide can be enhanced by emulsifying the second agent and controlling its average particle diameter at a fixed level or more.

Another aspect of the present invention is to provide a second agent having excellent stability of hydrogen peroxide, the second agent being an emulsion to be filled in a double structure container, in which a first inner container for filling a first agent and a second inner container for filling a second agent in the same outer container, and discharged and used. In addition, a still another aspect of the present invention is to provide an aerosol-type hair cosmetic material composition and an aerosol-type hair cosmetic material product, each of which is constituted to include the second agent.

Now, in view of the mechanism of the above-described separate filling/same pressure discharge-type double structure container, it is necessary to robustly constitute the outer container having a propellant sealed therein. In addition, if the reduced states of the first agent and the second agent filled in the bag-like bodies are viewable from the outside, such is convenient for a user, and hence, the outer container is constituted of, for example, a hard and transparent plastic material. Meanwhile, for the bag-like bodies for filling the first agent and the second agent, it is necessary to use a material that is relatively soft and readily deformable by pressurization such that the contents are surely discharged by a pressure of the propellant for pressurization.

In consequence, in many cases, the outer container and the bag-like bodies differ from each other in terms of impact resistance strength. For that reason, when the double structure container receives a large impact, for example, in the case where a double structure container is dropped from a hand during the use and collided on a hard floor surface, there may be a possibility that the bag-like bodies filled with the first agent and the second agent, respectively are broken. In addition, the liquid tightness (seal) in a connection portion between the opening of the above-described bag-like body and the discharge passage of the lid becomes loose, resulting in a possibility that the first agent or second agent filled in the bag-like body leaks out.

In a hair cosmetic material, such as an oxidation hair dyeing agent or a hair bleaching agent, by compounding an alkali agent and an oxidizing agent in the first agent and the second agent, respectively and uniformly mixing the both agents at the time of application to the hair, a hair dyeing effect or a hair bleaching effect is increased. Then, on the occasion of mixing the first agent and the second agent, a reactive gas, such as an oxygen gas (O₂), carbon dioxide, an ammonia gas, etc., is generated. In the case where the first agent and the second agent leak out due to the breakage of the above-described bag-like bodies, the both agents come into contact with each other to cause mixing to some extent, too. In consequence, the reactive gas is generated in the inside in the outer container, the amount of which is, however, small as compared with the case of artificially uniformly mixing the both agents at the time of application to the hair.

A yet another aspect of the present invention is to inhibit the generation of a reactive gas, such as an oxygen gas, etc., even when under special conditions that the first agent and the second agent leak out in the inside of the outer container from the bag-like bodies of the above-described double structure container, the first agent and the second agent come into contact with each other.

In a process of pursuing means for solving the foregoing problems, the inventors of the present application have obtained the following three findings.

(1) With respect to the generation of a reactive gas due to “intermixing” of the first agent and the second agent, it is important to consider intermixing of the both agents in a macroscopic meaning and intermixing in a microscopic meaning, namely mutual invasion or diffusion on the contact boundary between the both agents.

It is to be noted that the “intermixing” of the first agent and the second agent as referred to in the specification of the present application refers to both meanings including the intermixing in a macroscopic meaning and the intermixing in a microscopic meaning as described above, unless otherwise specifically indicated.

(2) In inhibiting the above-described intermixing, it is effective to regulate the viscosity of each of the first agent and the second agent within a certain range, and in order to achieve this, it is effective to control the content of the surfactant, the oily component, or the higher alcohol in the both agents.

(3) For the purpose of solving the problem under special conditions for “preventing an increase of a gas internal pressure of a small-capacity outer container”, the foregoing means are useful; however, its effect for inhibiting mixing properties is an extent of not giving an influence so much against artificial uniform mixing of the first agent and the second agent at the time of application to the hair.

Now, the separate filling/same pressure discharge-type double structure container is generally designed so as to simultaneously discharge the same amounts of the first agent and the second agent filled in the bag-like bodies. The first agent and the second agent to be filled therein are also discharged in the same amounts in amass ratio of 1/1 and then mixed, followed by application to the hair.

However, the separate filling/same pressure discharge-type double structure container has a mechanism so as to simultaneously discharge the first agent and the second agent by the same discharge pressure of the propellant. Therefore, in particular, in the case where the first agent and the second agent of the hair cosmetic material are a creamy preparation having a relatively high viscosity, unless properly regulating rheology properties (discharge properties by flowing at the time of pressurization) of each of the first agent and the second agent, actually, the equal amount discharge of the first agent and the second agent cannot be realized. In the case where the equal amount discharge properties are impaired to some degree or more, a commercial value itself of the hair cosmetic material product in which the first agent and the second agent are filled in the double structure container is affected.

Furthermore, in the case where the hair cosmetic material is, for example, a hair dyeing agent, such as a two-agent type oxidation hair dyeing agent or hair bleaching agent, etc., at least an alkali agent (furthermore, an oxidation dye) is compounded in the first agent, and an oxidizing agent is compounded in the second agent. Under such a restriction in view of composition, in order to achieve the equal amount discharge, it is not easy to decide what kind of category of rheology properties to be focused on and to grasp realization of the rheology properties through what kind of composition design of the first agent and the second agent.

The foregoing PTLs 6 and 7 are aimed to realize rheology properties with respect to a cosmetic material composition. However, PTL 6 discloses that with respect to a two-agent type hair dyeing agent to be discharged in a foam state from an aerosol foamer container, the dynamic viscoelasticity of a foam after discharge and mixing is regulated from the viewpoints of permeability after application to the hair, prevention of dripping from the hair, and the like. In addition, PTL 7 discloses that on review of spreading of a skin cosmetic material onto a skin at the time of application, or the like, an average emulsion particle diameter in an oil-in-water emulsified cosmetic material is regulated, thereby providing the cosmetic material with certain rheology properties. In consequence, PTLs 6 and 7 are not a good guide at all to realization of the above-described equal amount discharge in view of not only category of rheology properties but also composition design of the first agent and the second agent for achieving that.

A further aspect of the present invention is to provide a hair cosmetic material capable of performing equal amount discharge of a first agent and a second agent by a separate filling/same pressure discharge-type double structure container and provided with rheology properties with which the equal amount discharge properties can be kept with time.

### Solution to Problem and Advantageous Effects of Invention

A first aspect of the invention of the present application is concerned with an aerosol-type hair cosmetic material composition that is an oxidation hair dyeing agent or a hair bleaching agent to be used after being filled in a double structure container.

This double structure container includes a first inner container and a second inner container, each of which is independently provided, and an outer container for accommodating the first inner container and the second inner container therein.

The first inner container is one in which a first agent containing an alkali agent is filled. The second inner container is one in which a second agent containing an oxidizing agent is filled.

A space between the outer container and each of the first inner container and the second inner container is a propellant filling space for filling a propellant.

At least one of the first inner container and the second inner container is a pouch container formed by sticking periphery of sheet materials having a single-layer or multilayer structure including a resin layer together, and an innermost layer of the pouch container is a polyolefin resin layer.

The aerosol-type hair cosmetic material composition is a composition in which the agent to be filled in the pouch container contains a nonionic surfactant and a higher alcohol and satisfies a condition of a content ratio of the nonionic surfactant to the higher alcohol of 0.07 to 1.8 on amass ratio basis.

According to the aerosol-type hair cosmetic material composition as disclosed in the present application, a residual amount in the pouch container can be lessened.

Another aspect of the invention of the present application is concerned with an aerosol-type hair cosmetic material product constituted to include the aerosol-type hair cosmetic material composition as set forth in the foregoing first aspect; and a double structure container in which a first inner container for filling the first agent and a second inner container for filling the second agent, each of which is independently provided, are accommodated in the same outer container, a space between the outer container and each of the inner containers is a propellant filling space for filling a propellant, and a mechanism for simultaneously discharging the first agent and the second agent is provided.

At least one of the first inner container and the second inner container is a pouch container formed by sticking periphery of sheet materials having a single-layer or multilayer structure including a resin layer together, and an innermost layer of the pouch container is a polyolefin resin layer.

According to the aerosol-type hair cosmetic material composition as disclosed in the present application, a residual amount in the pouch container can be lessened.

A still another aspect of the invention of the present application is concerned with a second agent of an aerosol-type hair cosmetic material composition constituted to include a first agent containing an alkali agent and the second agent that is an emulsion containing hydrogen peroxide.

The second agent is used after being filled in the following double structure container. In this double structure container, a first inner container for filling the first agent and a second inner container for filling the second agent, each of which is independently provided, are accommodated in the same outer container. A space between the outer container and each of the inner containers is a propellant filling space for filling a propellant. The double structure container is provided with a mechanism for simultaneously discharging the first agent and the second agent.

The outer container and the second inner container are constituted to include a light-permeable material, and the inside of the second inner container can be visually recognized from the outside of the outer container.

The second agent has an average emulsion particle diameter of 1 μm or more.

The second agent may contain 1% by mass or more of a higher alcohol having 12 to 22 carbon atoms.

Furthermore, an aerosol-type hair cosmetic material composition constituted to include a first agent containing an alkali agent and the second agent may also be provided.

Moreover, an aerosol-type hair cosmetic material product constituted to include such an aerosol-type hair cosmetic material composition and a double structure container. In this double structure container, a first inner container for filling the first agent and a second inner container for filling the second agent, each of which is independently provided, are accommodated in the same outer container, a space between the outer container and each of the inner containers is a propellant filling space for filling a propellant, and a mechanism for simultaneously discharging the first agent and the second agent is provided.

The outer container and the second inner container are constituted to include a light-permeable material, and the inside of the second inner container can be visually recognized from the outside of the outer container.

The second agent as disclosed in the present application is excellent in stability of hydrogen peroxide even under irradiation with sunlight. Therefore, the second agent is suitable for use after being filled in a double structure container in which its residual amount is viewable. In addition, an aerosol-type hair cosmetic material composition and an aerosol-type hair cosmetic material product, each of which is constituted to include the second agent, can be obtained.

A yet another aspect of the invention of the present application is concerned with a hair cosmetic material including a first agent containing an alkali agent and a second agent containing an oxidizing agent. The first agent and the second agent are used in a double structure container provided with a mechanism of separating the first agent and the second agent from each other and simultaneously discharging the both agents by the above-described propellant. In this double structure container, a space for filling the first agent and a space for filling the second agent are each independently provided in the inside of a propellant filling space having a propellant for pressurization filled therein. The first agent and the second agent are each filled in the space for filling the respective agents.

Each of the first agent and the second agent is discharged in a liquid state; the first agent contains an alkali agent, whereas the second agent contains an oxidizing agent; and each of the first agent and the second agent has a viscosity falling within the range of from 7,000 to 30,000 mPa·s at 25° C.

Such a hair cosmetic material includes a first agent and a second agent, and the first agent contains an alkali agent, whereas the second agent contains an oxidizing agent. Then, the first agent and the second agent are respectively filled in a space for filling the first agent and a space for filling the second agent (for example, two bag-like bodies), each of which is independently provided, in a propellant filling space (inner space of the outer container) of the double structure container. In consequence, if a strong impact is applied to the double structure container, the generation of a reactive gas, such as an oxygen gas, etc., may possibly occur due to the contact between the first agent and the second agent, each of which has leaked out into the propellant filling space from each of the space for filling the first agent and the space for filling the second agent.

However, on that occasion, it has been noted that when the viscosity of each of the first agent and the second agent is designed to be 7,000 mPa·s or more at 25° C., the generation of a reactive gas which causes a gas internal pressure in the propellant filling space to be excessively increased is effectively inhibited. A reason for this may be considered to reside in the matter that if the viscosity of each of the first agent and the second agent is 7,000 mPa·s or more, the intermixing of the first agent and the second agent is inhibited, whereby a reactive gas is not generated to such an extent that the gas internal pressure in the propellant filling space is excessively increased. Moreover, in this case, an effect for inhibiting the intermixing of the first agent and the second agent is in such an extent that artificial uniform mixing of the first agent and the second agent at the time of application to the hair is not so much affected.

From the standpoint of ensuring the foregoing effect, an upper limit value of the viscosity of each of the first agent and the second agent at 25° C. is not particularly limited. However, from the points of view of achieving smooth discharge of the first agent and the second agent from the double structure container and making artificial mixing after discharge and before application to the hair easier, the upper limit value of the viscosity of each of the first agent and the second agent is set to 30,000 mPa·s.

In such a hair cosmetic material, each of the first agent and the second agent may contain at least one surfactant, and a content of the surfactant of each of the agents may be 10% by mass or less.

When the first agent and the second agent leak out, as factors of controlling the easiness of intermixing of the both agents, in addition to the viscosities, surface tensions of the both agents are exemplified. If the surface tensions are low, the first agent and the second agent are easily intermixed with each other. The content of the surfactant in each of the first agent and the second agent of the hair cosmetic material is 10% by mass or less. In consequence, lowerings of the surface tensions of the both agents are suppressed, and the first agent and the second agent, each of which has leaked out into the propellant filling space, are hardly intermixed with each other, and hence, the generation of an oxygen gas is inhibited. This effect does not affect so much the artificial uniform mixing of the first agent and the second agent at the time of application to the hair.

From the standpoint of ensuring such an effect, a lower limit value of the content of each of the surfactants in the first agent and the second agent is not particularly limited. However, from the point of view of emulsion stability, it is preferred that the content of the surfactant in each of the both agents is, for example, 1.5% by mass or more.

In addition, in such a hair cosmetic material, each of the first agent and the second agent may contain at least one oily component, and a total content of the oily components in the first agent and the second agent relative to a total amount of the first agent and the second agent may be 10% by mass or less.

It is known that the surface tension of the composition is lowered by the oily component. The total content of the oily components contained in the first agent and the second agent of the hair cosmetic material is 10% by mass or less relative to the total amount of the first agent and the second agent. For this reason, lowerings of the surface tensions of the both agents are suppressed, and the first agent and the second agent, each of which has leaked out into the propellant filling space, are hardly intermixed with each other, and hence, the generation of an oxygen gas is inhibited. This effect does not affect so much the artificial uniform mixing of the first agent and the second agent at the time of application to the hair.

Furthermore, in such a hair cosmetic material, each of the first agent and the second agent may contain at least one higher alcohol, and a total value of the following higher alcohol indexes regarding the higher alcohol contained in each of the agents may be 140 or less.

The higher alcohol index as referred to herein is an integrated value (a×b) of a carbon number (a) of the higher alcohol and a content value (b) in the first agent or the second agent of the higher alcohol in terms of a mass % unit. This higher alcohol is a monohydric alcohol having 12 or more and 22 or less carbon atoms, which is a linear or branched, saturated or unsaturated alcohol.

Although the higher alcohol may be generally considered to be one kind of oily components, in the present invention, the higher alcohol and the oily component are distinguished from each other. The content of the higher alcohol influences the surface tension of the composition, and such an influence also varies with the carbon number (molecular weight) of the higher alcohol. Then, the inventors of the present application thought a concept of “higher alcohol index” as an index of evaluating the influence of the higher alcohol against the inhibition of the generation of an oxygen gas (inhibition of lowerings of the surface tensions of the first agent and the second agent).

The total value of the higher alcohol indexes regarding the higher alcohol to be contained in each of the first agent and the second agent of the hair cosmetic material is 140 or less. For this reason, lowerings of the surface tensions of the first agent and the second agent are suppressed. Therefore, the first agent and the second agent, each of which has leaked out into the propellant filling space, are hardly intermixed with each other, and hence, the generation of an oxygen gas is inhibited. This effect does not affect so much the artificial uniform mixing of the first agent and the second agent at the time of application to the hair.

In addition, a hair cosmetic material product which is constituted to include such a hair cosmetic material and a double structure container provided with a mechanism of separating the first agent and the second agent from each other and simultaneously discharging the both agents by the above-described propellant may also be provided. In this double structure container, a space for filling the first agent and a space for filling the second agent are each independently provided in the inside of a propellant filling space having a propellant for pressurization filled therein. The first agent and the second agent of the hair cosmetic material are each filled in the space for filling the respective agents.

It is to be noted that in the case where the hair cosmetic material is constituted to include a third agent in a powder state or the like, the hair cosmetic material product may also include, in addition to the double structure container having the first agent and the second agent filled therein, the third agent attached thereto.

A hair cosmetic material product in which a first agent and a second agent of a hair cosmetic material are filled in a space for filling the first agent and a space for filling the second agent, respectively in a double structure container is provided. In this hair cosmetic material product, the generation of an oxygen gas due to the contact between the first agent and the second agent, which have leaked out from the space for filling the first agent and the space for filling the second agent, respectively in the double structure container, is effectively inhibited.

A further aspect of the invention of the present application is concerned with a hair cosmetic material including a first agent and a second agent.

The first agent and the second agent are each an agent to be used in a double structure container. In this double structure container, a space for filling the first agent and a space for filling the second agent are each independently provided in the inside of a propellant filling space having a propellant for pressurization filled therein, and a mechanism of separating the first agent and the second agent which are each filled in the space for filling the respective agents, from each other and simultaneously discharging the both agents by the above-described propellant is provided.

Both of the first agent and the second agent are discharged in a cream state and have a viscosity ratio V₃₀/V₁₂ of 0.5 or more, the ratio being a ratio of a viscosity V₃₀ at 30 rpm to a viscosity V₁₂ at 12 rpm as measured by using a B-type rotational viscometer under a condition at 25° C.

The viscosity ratio V₃₀/V₁₂(f) in the first agent and the viscosity ratio V₃₀/V₁₂(s) in the second agent falls within the range not exceeding 1.3 times each other.

In view of making it possible to perform equal amount discharge of a first agent and a second agent by a separate filling/same pressure discharge-type double structure container and realizing rheology properties such that the equal amount discharge properties can be kept with time, the inventors of the present application paid attention to three factors of a yield value, a strain γ, and a relaxation time λ(θ) regarding each of the first agent and the second agent as a fluid. The yield value is a fluidity factor of the fluid, and when this value is small, it becomes difficult to control the equal amount discharge. When a value of the strain γ is small, it contributes to an enhancement of the equal amount discharge properties with time. When relaxation time λ(θ) is short, it contributes to an enhancement of the equal amount discharge properties because the resultant is liable to return to the original fluid structure after stress relaxation.

It is to be noted that the three factors of a yield value, a strain γ, and a relaxation time λ(θ) were measured by using a titanium-made parallel plate-type viscoelasticity analyzer, RheoStress RS600 (available from HAAKE) at a measurement temperature of 25° C. and a measurement gap of 0.052 mm in a measurement mode of CS flow curve (stress: 0.01 to 300 Pa, 300 s) and creep (0.5 Pa, 60 s)/recovery (0 Pa, 60 s).

Then, as for a specific evaluation index of the rheology properties for which these respective factors are comprehensively satisfied, it has been found that a viscosity ratio V₃₀/V₁₂ of a viscosity V₃₀ at 30 rpm to a viscosity V₁₂ at 12 rpm regarding each of the first agent and the second agent as measured by using a B-type rotational viscometer is a suitable index. That is, it has been found that in the case where not only each of the viscosity ratio V₃₀/V₁₂(f) in the first agent and the viscosity ratio V₃₀/V₁₂(s) in the second agent is a fixed value or more, but also the viscosity ratios of the both agents fall within a fixed approximated numerical value range, the equal amount discharge of the first agent and the second agent by the separate filling/same pressure discharge-type double structure container can be realized.

In this aspect of the present application, with respect to the creamy first agent and second agent, each of which is filled in the double structure container, not only each of the viscosity ratio V₃₀/V₁₂(f) in the first agent and the viscosity ratio V₃₀/V₁₂(s) in the second agent is regulated to 0.5 or more, but also the viscosity ratios of the both agents are allowed to fall within the range not exceeding 1.3 times each other. Therefore, the equal amount discharge of the first agent and the second agent by the separate filling/same pressure discharge-type double structure container can be realized.

It is to be noted that the “equal amount discharge” as referred to in the present invention refers to the matter that an error in the discharge amount between the first agent and the second agent, each of which is filled in the double structure container, is preferably within 25% by mass, and more preferably within 20% by mass in the comparison in terms of the number of mass parts as a unit.

In such a hair cosmetic material, the first agent may contain at least one nonionic surfactant in an amount falling within the range of from 1 to 10% by mass in total.

In the product having the first agent and the second agent filled in the double structure container, taking into consideration a storage/circulation period from the time of production up to the time of sale, or a use period for which a user uses the hair cosmetic material while discharging the first agent and the second agent little by little, it is demanded to keep the equal amount discharge properties with time. That is, it is demanded to keep specified rheology properties with time regarding the first agent and the second agent.

In a process of pursuing means for coping with such a demand, the inventors of the present application have first found that keeping of the rheology properties with time is problematic especially in the first agent. As for a reason for this, it may be considered that in the case where the hair cosmetic material is an oxidation hair dyeing agent, the matter that the first agent contains a salt, such as an oxidation dye, etc., is related. Then, it has been found that if a nonionic surfactant is contained within a certain amount range in the first agent, the emulsion stability of the creamy first agent is enhanced, and hence, it becomes easy to keep the rheology properties with time.

When the first agent of the hair cosmetic material contains at least one nonionic surfactant in an amount falling within the range of from 1 to 10% by mass in total, the emulsion stability of the first agent is enhanced, the rheology properties are easily kept with time, and in its turn, the equal amount discharge properties of the first agent and the second agent with time are easily kept.

It is to be noted that the nonionic surfactant is stable with time in the first agent. In addition, when the nonionic surfactant is compounded in the first agent, it is effective for the emulsion stability of the creamy first agent especially in the case where the hair cosmetic material is a hair dyeing agent.

In addition, in such a hair cosmetic material, the first agent may further contain at least one oily component in an amount of 1% by mass or more in total.

Since the first agent of such a hair cosmetic material further contains at least one oily component in an amount of 1% by mass or more in total, in the case where the hair cosmetic material is a hair dyeing agent, its hair dyeing power can be ensured well.

This effect is ensured especially preferably in the case where the oily component is a hydrocarbon or an ester.

Furthermore, in such a hair cosmetic material, the first agent may further contain at least one higher alcohol, and a ratio C(n)/C(a) of a total content C(n) of the nonionic surfactant to a total content C(a) of the higher alcohol in the first agent in terms of a mass % unit may fall within the range of from 0.3 to 1.0.

With respect to keeping of the rheology properties with time in the first agent, it has been noted that the matter that the ratio C(n)/C(a) of a total content C(n) of the nonionic surfactant to a total content C(a) of the higher alcohol in the first agent falls within a fixed range is especially preferred.

Since the first agent further contains at least one higher alcohol, and the ratio C(n)/C(a) falls within the range of from 0.3 to 1.0 in terms of a mass % unit, the rheology properties of the first agent is kept with time, and in its turn, the equal amount discharge properties with time of the first agent and the second agent are kept.

In addition, in such a hair cosmetic material, the first agent may further contain at least one ionic surfactant, and a ratio C(i)/C(n) of a total content C(i) of the ionic surfactant to a total content C(n) of the nonionic surfactant in the first agent in terms of a mass % unit may be 1.5 or less.

Since the first agent of such a hair cosmetic material further contains at least one ionic surfactant, and the ratio C(i)/C(n) of a total content C(i) of the ionic surfactant to a total content C(n) of the nonionic surfactant in the first agent in terms of a mass % unit is 1.5 or less, keeping of the rheology properties with time in the first agent is especially excellent.

Furthermore, a hair cosmetic material product constituted to include such a hair cosmetic material and a double structure container may also be provided.

In this double structure container, a space for filling a first agent and a space for filling a second agent are each independently provided in a propellant filling space having a propellant for pressurization filled therein, and a mechanism of separating the first agent and the second agent, which are each filled in the space for filling the respective agents, from each other and simultaneously discharging the both agents by the above-described propellant is provided.

The first agent and the second agent of the hair cosmetic material are respectively filled in the space for filling the first agent and the space for filling the second agent in the double structure container.

In this hair cosmetic material product, it is possible to achieve the equal amount discharge of the first agent and the second agent by the double structure container, and the equal amount discharge properties with time can be kept.

### BRIEF DESCRIPTION OF DRAWINGS

FIG. 1 is a front view of an aerosol container in which a first inner container and a second inner container are existent in the same outer container in a first embodiment and a second embodiment of the present invention.

FIG. 2 is a front view showing an example of a double structure container in a third embodiment and a fourth embodiment of the present invention.

### REFERENCE SIGNS LIST

1: Double structure container

2: First inner container

3: Second inner container

4: Outer container

5: Valve unit

6: Actuator

7, 8: Discharge hole

9: Propellant filling space

10: Opening

11, 12: Cylindrical stem

13: Opening

### DESCRIPTION OF EMBODIMENTS

The inventions as disclosed in the present application are hereunder explained inclusive of best embodiments thereof.

### First Embodiment

An aerosol-type hair cosmetic material composition as disclosed in the present application is concerned with an oxidation dyeing agent or a hair bleaching agent, which is constituted to include a first agent containing an alkali agent and a second agent containing an oxidizing agent. The aerosol-type hair cosmetic material composition is used after being filled in a double structure container as described later.

The aerosol-type hair cosmetic material composition can be used according to the conventional procedure. The aerosol-type hair cosmetic material composition is preferably used for a human hair. In general, the aerosol-type hair cosmetic material composition is used by discharging the first agent and the second agent from a double structure container as described later at the time of use, applying to the hair, and after the treatment, washing away. A timing of mixing of the first agent and the second agent can be properly selected. The mixing may be performed before application to the hair; the application to the hair and the mixing may be simultaneously performed by using a comb or the like; or the application to the hair may be performed after installing a mixing device in the double structure container and then mixing the first agent and the second agent.

### [First Agent]

The first agent contains an alkali agent. Examples of the alkali agent include ammonia, alkanolamines (e.g., monoethanolamine, diethanolamine, triethanolamine, monoisopropanolamine, diisopropanolamine, triisopropanol-amine, etc.), organic amines (e.g., 2-amino-2-methyl-1-propanol, 2-amino-2-methyl-1,3-propane-diol, guanidine, etc.), basic amino acids (e.g., arginine, lysine, etc.) and salts thereof, inorganic alkalis (e.g., sodium hydroxide, potassium hydroxide, sodium carbonate, potassium carbonate, etc.), and the like.

The first agent may contain one or two or more alkali agents. It is preferred to set a content of the alkali agent to an amount such that a pH of the first agent falls within the range of from 8 to 12.

In the case where the aerosol-type hair cosmetic material composition is an oxidation hair dyeing agent composition, the first agent contains an oxidation dye.

The oxidation dye is a compound which can develop a color by oxidation polymerization. Examples of the oxidation dye include dye intermediates, couplers, melanin precursors, and the like. The first agent may contain one or two or more oxidation dyes.

More specifically, examples of the oxidation dye include phenylenediamine and derivatives thereof, phenol derivatives, aminophenol and derivatives thereof, diphenylamine and derivatives thereof, pyridine derivatives, pyrimidine derivatives, pyrazole derivatives, pyrrolidine derivatives, toluene derivatives, indole derivatives, pyrrole derivatives, imidazole derivatives, and the like.

Still more specifically, examples of the dye intermediate include phenylenediamines (provided that m-phenylenediamine is excluded), aminophenols (provided that m-aminophenol, 2,4-diaminophenol, and p-methylaminophenol are excluded), toluylenediamines (provided that toluene-3,4-diamine and toluene-2,4-diamine are excluded), diphenylamines, diaminophenylamines, N-phenylphenylene-diamines, diaminopyridines (provided that 2,6-diaminopyridine is excluded), and the like.

Examples of the coupler include pyrogallol, resorcin, catechol, m-aminophenol, m-phenylenediamine, 2,4-diaminophenol, 1,2,4-benzenetriol, toluene-3,4-diamine, toluene-2,4-diamine, hydroquinone, α-naphthol, 2,6-diaminopyridine, 1,5-dihydroxynaphthalene, 5-amino-o-cresol, p-methylaminophenol, 2,4-diaminophenoxy-ethanol, gallic acid, tannic acid, ethyl gallate, methyl gallate, propyl gallate, gobaishi, 5-(2-hydroxyethylamino)-2-methylphenol, and the like.

The first agent may contain a direct dye. By coloring the first agent itself, the first agent can be easily distinguished from the second agent. Examples of the direct dye include acid dyes, basic dyes, natural dyes, nitro dyes, disperse dyes, HC dyes, and the like.

The first agent may be compounded with, in addition to the above-described components, for example, water, such as purified water, distilled water, ion-exchanged water, etc., a water-soluble polymer, a solvent, a hydrocarbon, a wax, a higher fatty acid, an ester, a higher alcohol, a silicone, a nonionic surfactant, an anionic surfactant, a cationic surfactant, an ampholytic surfactant, a thickening agent, an amino acid, a saccharide, an antiseptic component, a chelating component, a pH adjuster component, an antioxidant, such as anhydrous sodium sulfite, etc., a plant or crude drug extract, a vitamin including an ascorbic acid, a perfume, a ceramide, an ultraviolet light absorber, an antistatic agent, a hair softener, a penetration enhancer, or the like.

It is preferred that the first agent is discharged as an emulsion from a double structure container as described later. Therefore, the first agent in a filled state in the first inner container is preferably an emulsion.

In addition, it is preferred that the first agent is discharged in a cream state from a double structure container as described later. A viscosity of the first agent in a cream state may be set to 5,000 to 50,000 mPa·s. The viscosity of the first agent is preferably 6,000 to 30,000 mPa·s. The viscosity is measured by using a B-type viscometer at 25° C. for one minute at a rotating rate of 12 rpm under conditions of using a No. 3 rotor (in the case where the viscosity is 5,000 mPa·s or less) or a No. 4 rotor (in the case where the viscosity is 5,000 mPa·s or more). As for the viscometer, for example, a VISCOMETER TV-10 viscometer may be used.

### [Second Agent]

The second agent contains an oxidizing agent. A content of the oxidizing agent in the second agent is preferably 0.1 to 15% by mass, more preferably 1.0 to 9.0% by mass, and still more preferably 2.0 to 6.0% by mass.

Examples of the oxidizing agent include hydrogen peroxide, urea peroxide, melamine peroxide, sodium percarbonate, potassium percarbonate, sodium perborate, potassium perborate, sodium peroxide, potassium peroxide, magnesium peroxide, barium peroxide, calcium peroxide, strontium peroxide, hydrogen peroxide adducts of sulfates, hydrogen peroxide adducts of phosphates, hydrogen peroxide adducts of pyrophosphates, and the like. The oxidizing agent maybe contained solely or in combination of two or more kinds thereof.

A pH of the second agent is preferably 2 to 6, and more preferably 3 to 5.

The second agent may be compounded with, for example, water, such as, purified water, distilled water, ion-exchanged water, etc., a water-soluble polymer, a higher alcohol, a nonionic surfactant, an anionic surfactant, a cationic surfactant, an ampholytic surfactant, a polyhydric alcohol, a hydrocarbon, a fat and oil, a wax, a higher fatty acid, an ester, an alkyl glyceryl ether, a solvent, a thickening agent, an amino acid, a silicone, a saccharide, phenoxyethanol, a hydrogen peroxide stabilizing component, such as, hydroxyethanediphosphonic acid, tetrasodium hydroxyethanedisulfonate, etc., a chelating component, a pH adjuster component, a plant or crude drug extract, a perfume, or the like.

It is preferred that the second agent is discharged as an emulsion from a double structure container as described later. Therefore, the second agent in a filled state in the second inner container is preferably an emulsion.

It is preferred that the second agent is discharged in a cream state from a double structure container as described later. A viscosity of the second agent in a cream state may be set to 5,000 to 50,000 mPa·s. The viscosity of the second agent is preferably 6,000 to 30,000 mPa·s.

### [Double Structure Container]

In the double structure container, a first inner container for filling the first agent and a second inner container for filling the second agent, each of which is independently provided, are accommodated in the same outer container, a space between the outer container and each of the inner containers is a propellant filling space for filling a propellant, and a mechanism for simultaneously discharging the first agent and the second agent is provided.

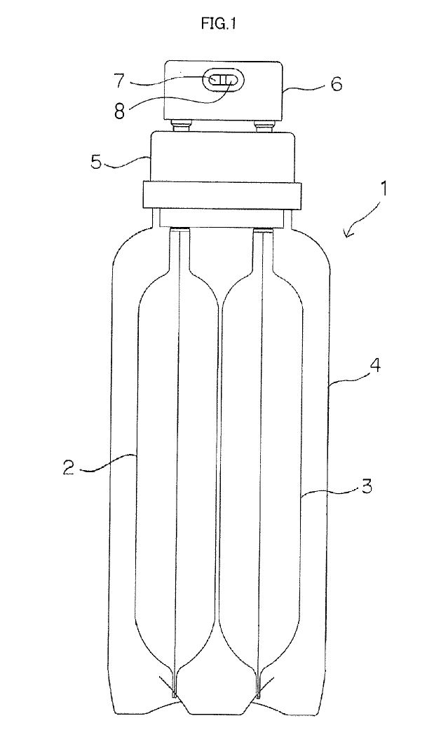

One specific example of the double structure container is explained by reference to FIG. 1.

In a double structure container 1, a first inner container 2 for filling the first agent and a second inner container 3 for filling the second agent are each independently provided and accommodated in the same outer container 4 in an approximately columnar shape. A space between the outer container 4 and each of the inner containers 2 and 3 is a propellant filling space for filling a propellant.

The double structure container 1 is provided with a valve unit 5. The valve unit 5 is provided with a first communication passage that is a passage of the first agent filled in the first inner container 2 and a second communication passage that is a passage of the second agent filled in the second inner container 3. In order to ensure each of the communication passages, for example, a rod-like joint material extending from a mouth portion of the inner container toward the inside of the container may be provided in the first inner container 2 and the second inner container 3.

In addition, with respect to each of the communication passages, the valve unit 5 is provided with a vertically movable valve stem for opening and closing each of the communication passages.

The valve unit 5 is coupled with an actuator 6, and when the actuator 6 is subjected to a press-down operation, each of the valve stems is opened. In the inside of the actuator 6, discharge holes 7 and 8 for discharging the first agent and the second agent, each of which has passed through the respective valve stem, are provided. The discharge holes 7 and 8 may be constituted so as to discharge separately the first agent and the second agent, or may be constituted such that the first agent and the second agent can be associated with each other in the actuator 6.

Therefore, when the actuator 6 is subjected to a press-down operation, the first agent filled in the first inner container 2 and the second agent filled in the second inner container 3 pass through the communication passages, the insides of the valve stems, and the discharge holes 7 and 8, respectively and are simultaneously discharged. In general, the valve stem in the valve unit 5 is pushed in the upper direction, and when the press-down operation of the actuator 6 is stopped, the valve stem is closed, whereby the discharge is stopped.

The inside of the outer container 4 is always in a pressurized state by a propellant. When the actuator 6 is subjected to a press-down operation, each of the inner containers 2 and 3 filled with the first agent and the second agent, respectively causes elastic deformation due to the pressure, whereby a filled material becomes possible to be discharged. In addition, a separately prepared mixing device may also be utilized.

The outer container 4 of the double structure container 1 is a pressure-resistant container constituted to include PET, polyacrylate, nylon, polypropylene, aluminum, tinplate, or the like. The outer container 4 in FIG. 1 is constituted of a light-permeable material.

All of the first inner container 2 and the second inner container 3 shown in FIG. 1 are pouch containers constituted by sticking periphery of elastic deformable sheet materials together, an innermost layer of which is a polyolefin resin layer. The pouch container is in a sheet-like shape in the case where the contents are not filled (or after the contents are completely discharged). The same phenomenon occurs even in the case where the joint material is inserted into the inner container.

The sheet material constituting the pouch container may be of either a single-layer structure or a multilayer structure. In addition, the pouch container may be constituted by sticking periphery of plural sheet materials together, or may be formed by folding a single sheet material, followed by sticking periphery of folded part together.

Examples of the polyolefin resin include PE, PP, and the like. In the case where the first agent contains an oxidation dye, it is preferred that the first inner container 2 is constituted to include a metal foil layer from the viewpoint of storage stability of the oxidation dye.

While illustration is omitted, any one of the first inner container 2 and the second inner container 3 may be a cylindrical container as disclosed in the foregoing PTLs 1 and 2. In addition, in any one of the first inner container 2 and the second inner container 3, the innermost layer of the pouch container may be made of a resin layer other than the polyolefin resin.

### [Propellant]

In the double structure container, the propellant filling space is separately provided independently of the space for filling the first agent and the space for filling the second agent.

As the propellant to be filled in the propellant filling space, for example, a liquefied gas or a compressed gas can be used. A compressed gas is preferred as the propellant from the viewpoint of safety.

Examples of the liquefied gas include LPG, DME, isopentane, and the like. Examples of the compressed gas include a nitrogen gas (compressed nitrogen), carbon dioxide, compressed air, and the like.

It is to be noted that when a carbonate, such as sodium carbonate, potassium carbonate, etc., is used as the alkali agent, and/or a percarbonate, such as sodium percarbonate, potassium percarbonate, etc., is used as the oxidizing agent, it is easy to generate carbon dioxide, thereby making it easy to prepare a foam dosage form after discharge.

### [Pouch Container Filler]

In the aerosol-type hair cosmetic material composition as disclosed in the present application, at least one of the first agent and the second agent is used after being filled in the pouch container, an innermost layer of which is a polyolefin resin layer. Preferably, the first agent is used after being filled in the pouch container, an innermost layer of which is a polyolefin resin layer. More preferably, both of the first agent and the second agent are respectively used after being filled in the pouch container, an innermost layer of which is a polyolefin resin layer.

For the sake of convenience of explanation, an agent which is used after being filled in the pouch container, an innermost layer of which is a polyolefin resin layer, is referred to as a pouch container filler. The pouch container filler is hereunder explained.

The pouch container filler contains a nonionic surfactant and a higher alcohol. Then, the pouch container filler satisfies a condition of a content ratio of the nonionic surfactant to the higher alcohol of 0.07 to 1.8 on a mass ratio basis. When the content ratio is less than 0.07 or more than 1.8, the residual amount in the pouch container becomes large. From the viewpoint of lessening the residual amount in the pouch container, the content ratio is allowed to fall preferably within the range of from 0.1 to 1.6, and more preferably within the range of from 0.3 to 1.5. From the viewpoint of not only lessening the residual in the pouch container but also making the discharge ratio keeping properties with time good, it is also preferred to allow the content ratio to fall within the range of 0.13 to 0.60.

A content of the nonionic surfactant in the pouch container filler can be set to 0.1 to 10% by mass, and it may also be set to 0.5 to 6% by mass.

A content of the higher alcohol in the pouch container filler can be set to 0.1 to 10% by mass, and it may also be set to 1 to 9% by mass.

Examples of the nonionic surfactant include polyoxyalkylene alkyl ethers, polyoxyalkylene fatty acid esters, alkyl polyglucosides, sugar esters, sugar amides, alkyl polyglyceryl ethers, and the like.

The pouch container filler contains, as the nonionic surfactant, preferably a polyoxyalkylene alkyl ether, and more preferably a polyoxyethylene (hereinafter also referred to as “POE”) alkyl ether. The alkyl moiety in the polyoxyalkylene alkyl ether preferably has 14 to 22 carbon atoms. Specific examples thereof include POE cetyl ether, POE oleyl ether, POE stearyl ether, POE behenyl ether, and the like.

From the viewpoint of emulsion stability of the pouch container filler, the pouch container filler contains a nonionic surfactant having an HLB value of 10 or more, and preferably having an HLB value of 14 or more. Furthermore, the pouch container filler contains, in addition to the foregoing nonionic surfactant, a nonionic surfactant having an HLB value of less than 10, and preferably having an HLB value of less than 8. It may be considered that when the emulsion is stable, the effects of the invention as disclosed in the present application are liable to be exhibited. The HLB value of the nonionic surfactant can be determined according to the known Griffin formula.

The higher alcohol is a monohydric alcohol having 6 or more carbon atoms. The higher alcohol is preferably saturated. In addition, the higher alcohol is preferably linear. In addition, the higher alcohol preferably has 12 to 22 carbon atoms.

From the viewpoint of making the discharge ratio keeping properties with time of the first agent and the second agent good, a content ratio of the higher alcohol having 14 to 18 carbon atoms to the whole of the higher alcohols in the pouch container filler is preferably set to 0.7 or more on a mass ratio basis.

From the viewpoint of making the discharge ratio keeping properties with time of the first agent and the second agent good, it is preferred that the pouch container filler contains a cationic surfactant. A content of the cationic surfactant in the pouch container filler is preferably 0.1 to 2.5% by mass, and more preferably 0.2 to 0.7% by mass.

From the viewpoint of an enhancement of brightness, it is preferred that the aerosol-type hair cosmetic material composition contains an oily component that is solid at 25° C.

Examples of the oily component that is solid at 25° C. include hydrocarbons, such as microcrystalline wax, etc.; waxes, such as lanolin, beeswax, candelilla wax, etc.; higher fatty acids, such as stearic acid, myristic acid, palmitic acid, behenic acid, etc.; esters composed of a monovalent fatty acid and a monohydric higher alcohol, such as stearyl stearate, myristyl myristate, cetyl palmitate, etc.; and the like. In the present application, silicones are not included in the oily component that is solid at 25° C.

It is preferred that the aerosol-type hair cosmetic material composition contains at least one of hydrocarbons and esters composed of a monovalent fatty acid and a monohydric higher alcohol as the oily component that is solid at 25° C., exclusive of a higher alcohol.

From the viewpoint of an enhancement of brightness, a content of the oily component that is solid at 25° C., exclusive of a higher alcohol in the pouch container filler is preferably 0.01 to 8% by mass, and more preferably 0.5 to 5% by mass. When the content of the oily component that is solid at 25° C., exclusive of a higher alcohol is too large, there is a concern that slipperiness with the pouch container is lowered, and from such a viewpoint, it is preferred that the instant content falls within the foregoing range.

### [Hair Cosmetic Material Product]

The present application discloses a hair cosmetic material product constituted to include the above-described aerosol-type hair cosmetic material composition and the above-described double structure container.

The hair cosmetic material product may properly include other arbitrary constitution. For example, the hair cosmetic material product may be constituted to include a brush, a comb, a cup for mixing, a pair of gloves, a hair cap, and the like.

### EXAMPLES

Working examples of the first embodiment of the present application are hereunder described. It should be construed that the technical scope of the invention as disclosed in the present application is not limited to the following Examples.

First of all, tables are explained. A numerical value expressing each of contents in the tables is a mass % unit.

As to the nonionic surfactant, an HLB value is described. A parenthesis continuing from POE expresses the number of moles of POE added.

It is to be noted that microcrystalline wax, stearic acid, stearyl stearate, and lanoline are solid at 25° C. Vaseline is not solid at 25° C.

A first agent and a second agent of each of oxidation hair dyeing agent compositions according to Examples 1 to 24 and Comparative Examples 1 and 2 in the first embodiment were prepared according to the conventional procedure. Each of the agents was a creamy emulsion and had a viscosity falling within the range of from 5,000 to 50,000 mPa·s. It is to be noted that in Examples 14 to 24 and Comparative Examples 1 to 2, the same second agent as in Example 1 was used.

In the following tests, the double structure container shown in FIG. 1 was used. All of the first inner container and the second inner container were pouch containers, and the resin material constituting the innermost layer of each of the pouch containers was polyethylene. Compressed nitrogen was used as the propellant.

The oxidation hair dyeing agent composition according to each of the Examples or each of the Comparative Examples was filled in the double structure container, a product was produced such that an internal pressure of the double structure container was 0.5 MPa, and the following tests were performed.

### [Evaluation Test of Residual Amount]

The oxidation hair dyeing agent composition was discharged until the filled materials could not be discharged from the product (whole amount discharge), and a weight of each of the first agent and the second agent within the inner containers after the whole amount discharge was measured.

With respect to the first agent and the second agent, a residual amount proportion in each of the inner containers was calculated according to the following formula.

Residual amount proportion (%)=(Weight after the whole amount discharge)/(Weight before the whole amount discharge)×100

The evaluation test of residual amount was performed thrice, and an average value thereof was determined and allowed to round off the second decimal place. The results are described in the tables.

In addition, the following criteria are set and described in the “Residual amount evaluation” row in the tables.

5: The residual amount proportion is less than 2%.

4: The residual amount proportion is 2% or more and less than 2.5%.

3: The residual amount proportion is 2.5% or more and less than 3%.

2: The residual amount proportion is 3% or more and less than 3.5%.

1: The residual amount proportion is 3.5% or more.

The pouch container according to each of the Examples was finely crushed to form a shape close to a single sheet, and its residual amount evaluation was good. On the other hand, as for each of the Comparative Examples, a condition of the ratio of the nonionic surfactant to the higher alcohol of 0.07 to 1.8 was not satisfied, and hence, it was considered that the residual amount evaluation was bad. Therefore, importance of the instant ratio in the residual amount evaluation was confirmed.

Next, as for the first agent of Example 20, the ratio of the nonionic surfactant to the higher alcohol was more than 1.6, and hence, it was considered that the residual amount evaluation was graded as 3. In addition, as for the first agent of Example 14, the instant ratio was less than 0.13, and hence, it was considered that the residual amount evaluation was graded as 4. That is, it was considered that by allowing the instant ratio to fall within the preferred range, the residual amount evaluation is enhanced.

As for the first agent of Example 9, though the oily component that is solid at 25° C., exclusive of the higher alcohol was not contained, the residual amount evaluation was graded as 5. On the other hand, even by compounding the oily component that is solid at 25° C., exclusive of the higher alcohol, a good residual amount evaluation is obtained. Therefore, it is able to realize an enhancement of the brightness by compounding the oily component that is solid at 25° C., exclusive of the higher alcohol while obtaining a good residual amount evaluation.

As for the first agent of Example 10, only the higher fatty acid was contained as the oily component that is solid at 25° C., exclusive of the higher alcohol, and hence, it was considered that the residual amount evaluation was graded as 4. From this evaluation, it was suggested that there is an oily component that is solid at 25° C., exclusive of the higher alcohol, which is preferred from the viewpoint of improving the residual amount evaluation.

### [Evaluation Test of Equal Amount Discharge Properties]

The product was stored at 25° C. for one month after its production, and thereafter, a discharge operation was continued until the residual amount (mass) of the second agent became half the filling amount. It is to be noted that after producing the product, the first agent and the second agent are discharged in substantially equal quantities.

At a point of time when the residual amount of the second agent became half the filling amount (at a point of time when a half of the second agent was discharged), the discharge amount of the first agent was measured, and a ratio to the discharge amount of the second agent was determined according to the following formula.

Discharge amount ratio of first agent to second agent=(Discharge amount of first agent at a point of time when a half of second agent was discharged)/(Half of second agent)

The closer to 1 the instant ratio, the better the equal amount discharge properties of the first agent and the second agent are.

The test was performed thrice, and an average value thereof was determined and allowed to round off the second decimal place, and the evaluation was made on the basis of the following criteria. The evaluation results are described in the “Equal amount discharge properties” row in the tables.

5: The ratio is 0.8 or more and 1.2 or less.

4: The ratio is 0.7 or more and less than 0.8, or more than 1.2 and 1.3 or less.

3: The ratio is 0.6 or more and less than 0.7, or more than 1.3 and 1.4 or less.

2: The ratio is 0.5 or more and less than 0.6, or more than 1.4 and 1.5 or less.

1: The ratio is less than 0.5, or more than 1.5.

All of the Examples were good in the evaluation. Therefore, in each of the Examples, the discharge ratio of the first agent and the second agent was kept good even after the storage.

It was revealed from Examples 14 to 20 that a ratio of the nonionic surfactant to the higher alcohol capable of making the equal amount discharge properties with time good exists.

As for the first agent of each of Examples 2 and 3 and the second agent of Example 11, a relatively large amount of the higher alcohol having 12 or less carbon atoms or 20 or more carbon atoms was contained relative to the whole of the higher alcohols in each of the agents, and hence, it was considered that the evaluation was graded as 3 to 4. That is, it was revealed that a preferred content ratio of the higher alcohol exists from the viewpoint of making the equal amount discharge properties with time good while keeping the good residual amount evaluation.

As for the second agent of Example 13, the nonionic surfactant having an HLB value of 10 or more is not contained, and hence, it was considered that the evaluation was graded as 4. That is, it was revealed that a preferred nonionic surfactant exists from the viewpoint of further making the equal amount discharge properties with time good while keeping the good residual amount evaluation.

As for the first agent of Example 8, only the anionic surfactant was contained as the ionic surfactant, and hence, it was considered that the evaluation was graded as 4. In addition, when referring to the evaluations of other Examples, it was considered that it is preferred to contain a cationic surfactant from the viewpoint of further making the equal amount discharge properties with time good while keeping the good residual amount evaluation.

### [Brightness]

The oxidation hair dyeing agent composition according to each of the Examples of the present application, which was discharged from the product and used for a human hair bundle according to the conventional procedure, realized a good brightness.

The first agent of Example 9 of the present application does not contain the oily component that is solid at 25° C., exclusive of the higher alcohol. Therefore, other Examples were higher in the brightness evaluation than Example 9.

TABLE 1

| | Example 1 | Example 2 | Example 3 | Example 4 | Example 5 | Example 6 | Example 7 |
|---|---|---|---|---|---|---|---|
| [First agent] | | | | | | | |
| Stearyl alcohol | 3 | 3 | 3 | 3 | 3 | 3 | 3 |
| Cetanol | 4 | | | 4 | 4 | 4 | 4 |
| Behenyl alcohol | | | 4 | | | | |
| Lauryl alcohol | | 4 | | | | | |
| POE(30) cetyl ether (HLB: 16.9) | 2 | 2 | 2 | | 2 | 3 | 2 |
| POE(50) oleyl ether (HLB: 17.6) | | | | 2 | | | |
| POE(2) cetyl ether (HLB: 5.4) | 1 | 1 | 1 | 1 | | | 1 |
| POE(2) stearyl ether (HLB: 5.0) | | | | | 1 | | |
| Stearyltrimethylammonium chloride | 1 | 1 | 1 | 1 | 1 | 1 | |
| Cetyltrimethylammonium chloride | | | | | | | 1 |
| Sodium laurylsulfate | | | | | | | |
| Vaseline | | | | | | | |
| Microcrystalline wax | 3 | 3 | 3 | 3 | 3 | 3 | 3 |
| Stearic acid | | | | | | | |
| Anhydrous sodium sulfite | 0.1 | 0.1 | 0.1 | 0.1 | 0.1 | 0.1 | 0.1 |
| p-Phenylenediamine | 0.5 | 0.5 | 0.5 | 0.5 | 0.5 | 0.5 | 0.5 |
| m-Aminophenol | 0.4 | 0.4 | 0.4 | 0.4 | 0.4 | 0.4 | 0.4 |
| Resorcin | 0.5 | 0.5 | 0.5 | 0.5 | 0.5 | 0.5 | 0.5 |
| Ascorbic acid | 0.2 | 0.2 | 0.2 | 0.2 | 0.2 | 0.2 | 0.2 |
| 28% Ammonia water | 4 | 4 | 4 | 4 | 4 | 4 | 4 |
| Purified water | Balance | Balance | Balance | Balance | Balance | Balance | Balance |
| Total | 100 | 100 | 100 | 100 | 100 | 100 | 100 |
| Innermost layer | PE | PE | PE | PE | PE | PE | PE |
| Nonionic surfactant/higher alcohol | 0.43 | 0.43 | 0.43 | 0.43 | 0.43 | 0.43 | 0.43 |
| Residual amount evaluation | 5 | 5 | 5 | 5 | 5 | 5 | 5 |
| Residual amount proportion (%) | 1.5 | 1 | 1.2 | 1.5 | 1.7 | 1.2 | 1.4 |
| [Second agent] | | | | | | | |
| 35% Hydrogen peroxide water | 16 | 16 | 16 | 16 | 16 | 16 | 16 |
| Stearyl alcohol | 4 | 4 | 4 | 4 | 4 | 4 | 4 |
| Cetanol | 3 | 3 | 3 | 3 | 3 | 3 | 3 |
| Arachyl alcohol | | | | | | | |
| Stearyl stearate | 2 | 2 | 2 | 2 | 2 | 2 | 2 |
| Lanoline | | | | | | | |
| Stearyltrimethylammonium chloride | 0.4 | 0.4 | 0.4 | 0.4 | 0.4 | 0.4 | 0.4 |
| POE(30) behenyl ether (HLB: 16.1) | 1 | 1 | 1 | 1 | 1 | 1 | 1 |
| POE(2) cetyl ether (HLB: 5.4) | | | | | | | |
| Sodium myristyl sulfate | 1 | 1 | 1 | 1 | 1 | 1 | 1 |
| Phenoxyethanol | 0.1 | 0.1 | 0.1 | 0.1 | 0.1 | 0.1 | 0.1 |
| Hydroxyethanediphosphonic acid | 0.2 | 0.2 | 0.2 | 0.2 | 0.2 | 0.2 | 0.2 |
| Phosphoric acid | Adjusted to a pH of 3 | | | | | | |
| Purified water | Balance | Balance | Balance | Balance | Balance | Balance | Balance |
| Total | 100 | 100 | 100 | 100 | 100 | 100 | 100 |
| Innermost layer | PE | PE | PE | PE | PE | PE | PE |
| Nonionic surfactant/higher alcohol | 0.14 | 0.14 | 0.14 | 0.14 | 0.14 | 0.14 | 0.14 |
| Residual amount evaluation | 5 | 5 | 5 | 5 | 5 | 5 | 5 |
| Residual amount proportion (%) | 1 | 1 | 1 | 1 | 1 | 1 | 1 |
| Equal amount discharge properties | 5 | 3 | 4 | 5 | 5 | 5 | 5 |

TABLE 2

| | Example 8 | Example 9 | Example 10 | Example 11 | Example 12 | Example 13 |
|---|---|---|---|---|---|---|
| [First agent] | | | | | | |
| Stearyl alcohol | 3 | 3 | 3 | 3 | 3 | 3 |
| Cetanol | 4 | 4 | 4 | 4 | 4 | 4 |
| Behenyl alcohol | | | | | | |
| Lauryl alcohol | | | | | | |
| POE(30) cetyl ether (HLB: 16.9) | 2 | 2 | 2 | 2 | 2 | 2 |
| POE(50) oleyl ether (HLB: 17.6) | | | | | | |
| POE(2) cetyl ether (HLB: 5.4) | 1 | 1 | 1 | 1 | 1 | 1 |
| POE(2) stearyl ether (HLB: 5.0) | | | | | | |
| Stearyltrimethylammonium chloride | | 1 | 1 | 1 | 1 | 1 |
| Cetyltrimethylammonium chloride | | | | | | |
| Sodium laurylsulfate | 1 | | | | | |
| Vaseline | | 3 | | | | |
| Microcrystalline wax | 3 | | | 3 | 3 | 3 |
| Stearic acid | | | 3 | | | |
| Anhydrous sodium sulfite | 0.1 | 0.1 | 0.1 | 0.1 | 0.1 | 0.1 |
| p-Phenylenediamine | 0.5 | 0.5 | 0.5 | 0.5 | 0.5 | 0.5 |
| m-Aminophenol | 0.4 | 0.4 | 0.4 | 0.4 | 0.4 | 0.4 |
| Resorcin | 0.5 | 0.5 | 0.5 | 0.5 | 0.5 | 0.5 |
| Ascorbic acid | 0.2 | 0.2 | 0.2 | 0.2 | 0.2 | 0.2 |
| 28% Ammonia water | 4 | 4 | 4 | 4 | 4 | 4 |
| Purified water | Balance | Balance | Balance | Balance | Balance | Balance |
| Total | 100 | 100 | 100 | 100 | 100 | 100 |
| Innermost layer | PE | PE | PE | PE | PE | PE |
| Nonionic surfactant/higher alcohol | 0.43 | 0.43 | 0.43 | 0.43 | 0.43 | 0.43 |
| Residual amount evaluation | 5 | 5 | 4 | 5 | 5 | 5 |
| Residual amount proportion (%) | 1.8 | 1.2 | 2.1 | 1.5 | 1.5 | 1.5 |
| [Second agent] | | | | | | |
| 35% Hydrogen peroxide water | 16 | 16 | 16 | 16 | 16 | 16 |
| Stearyl alcohol | 4 | 4 | 4 | 4 | 4 | 4 |
| Cetanol | 3 | 3 | 3 | | 3 | 3 |
| Arachyl alcohol | | | | 3 | | |
| Stearyl stearate | 2 | 2 | 2 | 2 | | 2 |
| Lanoline | | | | | 2 | |
| Stearyltrimethylammonium chloride | 0.4 | 0.4 | 0.4 | 0.4 | 0.4 | 0.4 |
| POE(30) behenyl ether (HLB: 16.1) | 1 | 1 | 1 | 1 | 1 | |
| POE(2) cetyl ether (HLB: 5.4) | | | | | | 1 |
| Sodium myristyl sulfate | 1 | 1 | 1 | 1 | 1 | 1 |
| Phenoxyethanol | 0.1 | 0.1 | 0.1 | 0.1 | 0.1 | 0.1 |
| Hydroxyethanediphosphonic acid | 0.2 | 0.2 | 0.2 | 0.2 | 0.2 | 0.2 |
| Phosphoric acid | Adjusted to a pH of 3 | | | | | |
| Purified water | Balance | Balance | Balance | Balance | Balance | Balance |
| Total | 100 | 100 | 100 | 100 | 100 | 100 |
| Innermost layer | PE | PE | PE | PE | PE | PE |
| Nonionic surfactant/higher alcohol | 0.14 | 0.14 | 0.14 | 0.14 | 0.14 | 0.14 |
| Residual amount evaluation | 5 | 5 | 5 | 5 | 5 | 5 |
| Residual amount proportion (%) | 1 | 1 | 1 | 0.9 | 1.2 | 1.2 |
| Equal amount discharge properties | 4 | 5 | 5 | 3 | 4 | 4 |

TABLE 3

| [First agent] | Example 14 | Example 15 | Example 16 | Example 17 | Example 18 | Example 19 | Example 20 |
|---|---|---|---|---|---|---|---|
| Stearyl alcohol | 4 | 4 | 2 | 3 | 2 | 2 | 2 |
| Cetanol | 5 | 5 | 2.5 | 4 | 3 | 2 | 2 |
| POE(30) cetyl ether (HLB: 16.9) | 0.6 | 2 | 2 | 3 | 3 | 3 | 4 |
| POE(2) cetyl ether (HLB: 5.4) | 0.3 | 1 | 1 | 1.7 | 2 | 3 | 3 |
| Stearyltrimethylammonium chloride | 1 | 1 | 1 | 1 | 1 | 1 | 1 |
| Microcrystalline wax | 3 | 3 | 3 | 3 | 3 | 3 | 3 |
| Anhydrous sodium sulfite | 0.1 | 0.1 | 0.1 | 0.1 | 0.1 | 0.1 | 0.1 |
| p-Phenylenediamine | 0.5 | 0.5 | 0.5 | 0.5 | 0.5 | 0.5 | 0.5 |
| m-Aminophenol | 0.4 | 0.4 | 0.4 | 0.4 | 0.4 | 0.4 | 0.4 |
| Resorcin | 0.5 | 0.5 | 0.5 | 0.5 | 0.5 | 0.5 | 0.5 |
| Ascorbic acid | 0.2 | 0.2 | 0.2 | 0.2 | 0.2 | 0.2 | 0.2 |
| 28% Ammonia water | 4 | 4 | 4 | 4 | 4 | 4 | 4 |
| Purified water | Balance | Balance | Balance | Balance | Balance | Balance | Balance |
| Total | 100 | 100 | 100 | 100 | 100 | 100 | 100 |
| Innermost layer | PE | PE | PE | PE | PE | PE | PE |
| Nonionic surfactant/higher alcohol | 0.10 | 0.33 | 0.67 | 0.67 | 1.00 | 1.50 | 1.75 |
| Residual amount evaluation | 4 | 5 | 5 | 5 | 5 | 5 | 3 |
| Residual amount proportion (%) | 2.4 | 1.8 | 1.2 | 1.6 | 1 | 1 | 2.5 |
| Equal amount discharge properties | 4 | 5 | 4 | 4 | 4 | 4 | 3 |

TABLE 4

| | | | | | Comparative | Comparative |
|---|---|---|---|---|---|---|
| [First agent] | Example 21 | Example 22 | Example 23 | Example 24 | Example 1 | Example 2 |
| Stearyl alcohol | 3 | 3 | 3 | 3 | 5 | 1.5 |
| Cetanol | 4 | 4 | 4 | 4 | 5 | 2 |
| POE(30) cetyl ether (HLB: 16.9) | 2 | 2 | 2 | 2 | 0.3 | 4 |
| POE(2) cetyl ether (HLB: 5.4) | 1 | 1 | 1 | 1 | 0.2 | 3 |
| Stearyltrimethylammonium chloride | 1 | 1 | 0.5 | 0.3 | 1 | 1 |
| Microcrystalline wax | 2 | 5 | 3 | 3 | 3 | 3 |
| Anhydrous sodium sulfite | 0.1 | 0.1 | 0.1 | 0.1 | 0.1 | 0.1 |
| p-Phenylenediamine | 0.5 | 0.5 | 0.5 | 0.5 | 0.5 | 0.5 |
| m-Aminophenol | 0.4 | 0.4 | 0.4 | 0.4 | 0.4 | 0.4 |
| Resorcin | 0.5 | 0.5 | 0.5 | 0.5 | 0.5 | 0.5 |
| Ascorbic acid | 0.2 | 0.2 | 0.2 | 0.2 | 0.2 | 0.2 |
| 28% Ammonia water | 4 | 4 | 4 | 4 | 4 | 4 |
| Purified water | Balance | Balance | Balance | Balance | Balance | Balance |
| Total | 100 | 100 | 100 | 100 | 100 | 100 |
| Innermost layer | PE | PE | PE | PE | PE | PE |
| Nonionic surfactant/higher alcohol | 0.43 | 0.43 | 0.43 | 0.43 | 0.05 | 2.00 |
| Residual amount evaluation | 5 | 5 | 5 | 5 | 1 | 2 |
| Residual amount proportion (%) | 1.2 | 1.9 | 1.6 | 1.6 | 3.6 | 3.2 |
| Equal amount discharge properties | 5 | 5 | 5 | 5 | 4 | 2 |

### Second Embodiment

An aerosol-type hair cosmetic material composition as disclosed in the second embodiment of the present application is constituted to include a first agent containing an alkali agent and a second agent that is an emulsion containing hydrogen peroxide.

### [First Agent]

The components of the first agent in the second embodiment are the same as those in the first agent in the first embodiment, and hence, their explanation is omitted.

It is preferred that the first agent is discharged in an emulsion state from a double structure container as described later. Therefore, the first agent in a filled state in the first inner container is preferably an emulsion.

In addition, it is preferred that the first agent is discharged in a cream state from a double structure container as described later. A viscosity of the first agent in a cream state may be set to 3,000 to 40,000 mPa·s. The viscosity is measured by using a B-type viscometer at 25° C. for one minute at a rotating rate of 12 rpm under conditions of using a No. 3 rotor (in the case where the viscosity is 5,000 mPa·s or less) or a No. 4 rotor (in the case where the viscosity is 5,000 mPa·s or more). As for the viscometer, for example, a VISCOMETER TV-10 viscometer may be used.

### [Second Agent]

The second embodiment discloses a second agent of an aerosol-type hair cosmetic material composition constituted to include a first agent containing an alkali agent and the second agent that is an emulsion containing hydrogen peroxide, wherein

the second agent is used after being filled in a double structure container in which a first inner container for filling the first agent and a second inner container for filling the second agent, each of which is independently provided, are accommodated in the same outer container; a space between the outer container and each of the inner containers is a propellant filling space for filling a propellant; the outer container and the second inner container are constituted to include a light-permeable material, and the inside of the second inner container can be visually recognized from the outside of the outer container; and a mechanism for simultaneously discharging the first agent and the second agent is provided, and

the second agent has an average emulsion particle diameter of 1 μm or more.

The second agent contains an oxidizing agent. In the second agent, hydrogen peroxide is an essential component. A content of the hydrogen peroxide in the second agent is preferably 0.1 to 15% by mass, more preferably 1.0 to 9.0% by mass, and still more preferably 2.0 to 6.0% by mass.

In addition, a pH of the second agent is preferably 2 to 6, and more preferably 3 to 5.

The second agent may contain, in addition to the above-described hydrogen peroxide, one or two or more appropriate oxidizing agents. Examples thereof include urea peroxide, melamine peroxide, sodium percarbonate, potassium percarbonate, sodium perborate, potassium perborate, sodium peroxide, potassium peroxide, magnesium peroxide, barium peroxide, calcium peroxide, strontium peroxide, hydrogen peroxide adducts of sulfates, hydrogen peroxide adducts of phosphates, hydrogen peroxide adducts of pyrophosphates, and the like.

The second agent is an emulsion, and hence, it is preferred that the second agent contains a surfactant and an oily component.

As the surfactant, cationic surfactants, anionic surfactants, nonionic surfactants, and ampholytic surfactants, such as cocamidopropyl betaine, etc., can be used.

Examples of the cationic surfactant include alkyltrimethylammonium salts, such as stearyltrimethylammonium chloride, lauryltrimethylammonium chloride, etc., alkenyltrimethylammonium salts, dialkyldimethylammonium salts, dialkenyldimethylammonium salts, alkyloyl amidopropyl dimethylamines, alkyl piperidinium salts, benzalkonium salts, and the like.

Examples of the anionic surfactant include alkyl sulfates, alkyl ether sulfates, such as sodium lauryl polyoxyethylene (hereinafter also referred to as “POE”) ether sulfate (sodium laureth sulfate), etc., alkenyl sulfates, alkenyl ether sulfates, alkane sulfonates, olefin sulfonate, phosphoric acid mono- or diester types, and the like.

Examples of the nonionic surfactant include polyoxyalkylene alkyl ethers, such as POE cetyl ether, POE behenyl ether, etc., polyoxyalkylene fatty acid esters, alkyl polyglucosides, sugar esters, sugar amides, alkyl polyglyceryl ethers, and the like.

From the viewpoint of stability with time of the average emulsion particle diameter, it is preferred that the second agent contains a nonionic surfactant.

The second agent preferably contains a POE alkyl ether that is the nonionic surfactant. The alkyl moiety constituting the POE alkyl ether preferably has 12 to 22 carbon atoms. In addition, the alkyl moiety constituting the POE alkyl ether is preferably linear.

From the viewpoint of obtaining a good average emulsion particle diameter in the second agent, it is preferred that not only the second agent contains a cationic surfactant and an anionic surfactant, but also of the both surfactants, the surfactant having a smaller content is controlled to a content of 0.01 to 0.5% by mass. More preferably, the surfactant having a smaller content is the cationic surfactant. It may be considered that by making a difference in the contents between the cationic surfactant and the anionic surfactant, good stability of hydrogen peroxide is obtained while making a complex formed by the both surfactants small.

The content of the surfactant in the second agent is preferably 0.1 to 10.0% by mass, more preferably 0.3 to 7.0% by mass, and still more preferably 0.5 to 5.0% by mass.

The content of the nonionic surfactant in the second agent may be 0.3% by mass or more, may also be 0.4 to 7% by mass, and may further be 0.5 to 5% by mass.

In the second agent, the content of the nonionic surfactant, an HLB value of which is 12 to 17, maybe less than 1.5% by mass, and may also be 1.3% by mass or less. The HLB value of the nonionic surfactant can be determined according to the known Griffin formula.

From the viewpoint of stability of hydrogen peroxide under irradiation with sunlight, it is preferred that the second agent contains 1% by mass or more of a higher alcohol having 12 to 22 carbon atoms. A content of the higher alcohol having 12 to 22 carbon atoms in the second agent may be 1 to 10% by mass, and may also be 3 to 8% by mass. It is to be noted that the higher alcohol is a monohydric alcohol.

In the foregoing PTL 4, the storage of the second agent under irradiation with sunlight is not discussed at all. In consequence, any disclosure of a technical finding that by paying attention to a specified higher alcohol and further specifying its content, the stability of hydrogen peroxide under irradiation with sunlight is enhanced is not provided in the foregoing PTL 4.

Examples of the higher alcohol having 12 to 22 carbon atoms include linear or branched, saturated or unsaturated aliphatic alcohols. Specifically, examples thereof include lauryl alcohol, myristyl alcohol, cetyl alcohol (cetanol), cetostearyl alcohol, stearyl alcohol, arachyl alcohol, behenyl alcohol, isostearyl alcohol, oleyl alcohol, 2-hexyldodecanol, 2-octyldodecanol, decyltetradecanol, linoleyl alcohol, linolenyl alcohol, lanolin alcohol, and the like. Of these, saturated higher alcohols, specifically lauryl alcohol, myristyl alcohol, cetyl alcohol, stearyl alcohol, arachyl alcohol, and behenyl alcohol, are preferred.

The second agent may contain, as the oily component, vaseline, microcrystalline wax, a hydrocarbon, such as liquid paraffin, etc., a fat and oil, a wax, a higher fatty acid, an ester, such as cetyl octanoate, stearyl stearate, etc., or the like.

As for a content of the oily component in the second agent, its upper limit value may be 10% by mass, and a content of the oily component exclusive of the higher alcohol may be 5% by mass or less. In the second agent, a content of the oily component that is liquid at 25° C., exclusive of the higher alcohol is preferably 5% by mass or less.

The second agent may be compounded with an appropriate arbitrary component in addition to the above-described components. For example, water, e.g., purified water, distilled water, ion-exchanged water, etc., a water-soluble polymer, a polyhydric alcohol, an alkyl glyceryl ether, a solvent, a thickening agent, an amino acid, a silicone, a saccharide, phenoxyethanol, a hydrogen peroxide stabilizing component, such as, hydroxyethanediphosphonic acid, tetrasodium hydroxyethanediphosphonate, etc., a chelating component; a pH adjuster component, a plant or crude drug extract; a vitamin including an ascorbic acid, a perfume, or the like, may be compounded as the arbitrary component.

The second agent has an average emulsion particle diameter of 1 μm or more. The average emulsion particle diameter is preferably 1 to 100 μm, more preferably 2 to 50 μm, and still more preferably 5 to 20 μm. When the average emulsion particle diameter of the second agent is less than 1 μm, the stability of hydrogen peroxide under irradiation with sunlight becomes insufficient.

In the present application, the average emulsion particle diameter is measured by using a laser diffraction scattering method particle size distribution measuring device (a trade name: MICROTRACMT3000II, available from Nikkiso Co., Ltd.), and a median diameter (d50 value) on a volume basis is measured. A device having the same quality in the foregoing laser diffraction scattering method particle size distribution measuring device may also be used.

The second agent is preferably in a cream state. In addition, it is preferred that the second agent is discharged in a cream state from a double structure container as described later. From the viewpoints of making mixing properties with the first agent good, making the mixture compatible with the hair, and well suppressing dripping of the mixture, a viscosity of the creamy second agent may be 3,000 to 40,000 mPa·s. In addition, from the viewpoint of stability of the emulsion particle diameter, the viscosity of the second agent is preferably 5,000 to 35,000 mPa·s, and more preferably 7,000 to 30,000 mPa·s.

### [Aerosol-Type Hair Cosmetic Material Composition]

The aerosol-type hair cosmetic material composition as disclosed in the present application is constituted to include the first agent and the second agent. The aerosol-type hair cosmetic material composition as disclosed in the present application may be a two-agent type, or may also be a multi-agent type including three or more agents.

The aerosol-type hair cosmetic material composition may be constituted to further include an oxidation aid, a treatment agent, and the like.

Example of the aerosol-type hair cosmetic material composition include an oxidation hair dyeing agent composition, a hair bleaching agent composition, a hair dedyeing agent composition, and the like. Preferably, the aerosol-type hair cosmetic material composition is utilized for treating a human hair.

The aerosol-type hair cosmetic material composition can be used according to the conventional procedure. In general, the aerosol-type hair cosmetic material composition is used by discharging the first agent and the second agent from a double structure container as described later at the time of use, applying the agents to the hair, and after the treatment, washing the agents away. A timing of mixing of the first agent and the second agent can be properly selected. The mixing may be performed before application to the hair; the application to the hair and the mixing may be simultaneously performed by using a comb or the like; or the application to the hair may be performed after installing a mixing device in the double structure container and performing mixing.

In the case where the aerosol-type hair cosmetic material composition is constituted to include an oxidation aid, the first to third agents are mixed at the time of mixing. In the case where the aerosol-type hair cosmetic material composition is constituted to include a treatment agent, the first agent, the second agent, and the treatment agent may be mixed at the time of mixing, or the treatment processing may be performed by using the treatment agent after the dyeing treatment/bleaching or the dedyeing treatment.

### [Double Structure Container]

In the double structure container, a first inner container for filling the first agent and a second inner container for filling the second agent, each of which is independently provided, are accommodated in the same outer container; a space between the outer container and each of the inner containers is a propellant filling space for filling a propellant; the outer container and the second inner container are constituted to include a light-permeable material, and the inside of the second inner container can be visually recognized from the outside of the outer container; and a mechanism for simultaneously discharging the first agent and the second agent is provided.

The double structure container in the second embodiment is the same as the double structure container in the first embodiment (see FIG. 1), and hence, its explanation is omitted.

As for the first inner container 2 and the second inner container 3, conventionally known containers (for example, a pouch, etc.) can be properly used.

The outer container 4 of the double structure container is a pressure-resistant container constituted of a light-permeable material, such as PET, polyacrylate, nylon, polypropylene, etc. Therefore, the inside of the outer container 4 is viewable. In the outer container 4, other sites than the site where the second inner container 3 can be visually recognized may be colored, or a packaging material may be installed so as to leave a site where the second inner container 3 can be visually recognized.

The second inner container 3 is placed in juxtaposition with the first inner container 2. The second inner container may be constituted by sticking periphery of elastic deformable sheets constituted of a light-permeable material, such as PET, PE, etc., together (the instant sheet will be hereinafter also referred to as “light-permeable sheet”). In addition, the second inner container 3 may be constituted by using an elastic deformable sheet constituted to include a light-impermeable material, such as aluminum, etc., for the side opposing the first inner container 2, using a light-permeable sheet for the reverse side opposing the outer container 4, and sticking these together. In addition, a specified portion of the light-permeable sheet may be colored, or a specified portion thereof may be decorated in a light-impermeable manner.

In the case where the first agent contains an oxidation dye, from the viewpoint of storage stability of the oxidation dye, it is preferred to use a pouch containing a metal foil as the first inner container 2. In this case, the first inner container 2 becomes light-impermeable. Therefore, the residual amount of the first agent in the first inner container 2 cannot be visually recognized.

In the light of the above, the double structure container 1 is constituted in such a manner that the inside of the second inner container 3 can be visually recognized. Therefore, in the case where the aerosol-type hair cosmetic material product is set aside in plural times and used, in particular, the residual amount of the second agent can be visually recognized easily. The first agent and the second agent are placed under the same pressure, and hence, the residual amount of the second agent becomes a yardstick for the residual amount of the first agent.

In addition, the double structure containers as disclosed in FIGS. 3 to 6 of the foregoing PTL 2 and FIG. 1 of the foregoing PTL 3 may also be used while referring the constitution of the double structure container as described above.

### [Propellant]

In the double structure container, the propellant filling space is separately provided independently of the space for filling the first agent and the space for filling the second agent.

As the propellant to be filled in the propellant filling space, for example, a liquefied gas or a compressed gas can be used. From the viewpoint of safety as well as the viewpoint that if the whole or a part of the outer container of the double structure container is made light-permeable, the propellant filling space may be visually recognized, a compressed gas is preferred as the propellant.

Examples of the liquefied gas include LPG, DME, isopentane, and the like. Examples of the compressed gas include a nitrogen gas, carbon dioxide, compressed air, and the like.

It is to be noted that in the case where it is contemplated to discharge a filled material in a foam state from the double structure container, a liquefied gas may be filled in the inner container. On the other hand, in the case of discharging a filled material in a cream state, a liquefied gas is not filled in the inner container, or a filling amount of a liquefied gas in the inner container is controlled at an extremely low level. When a carbonate, such as sodium carbonate, potassium carbonate, etc., is used as the alkali agent, and/or a percarbonate, such as sodium percarbonate, potassium percarbonate, etc., is used as the oxidizing agent, it is easy to generate carbon dioxide, thereby making a foam dosage form after discharge.

### [Aerosol-Type Hair Cosmetic Material Product]

The present application discloses an aerosol-type hair cosmetic material product constituted to include the aerosol-type hair cosmetic material composition and the double structure container.

The first agent and the second agent of the aerosol-type hair cosmetic material composition are filled in the double structure container. In the case where the aerosol-type hair cosmetic material composition includes an arbitrary constitution (for example, a third agent) in addition to the first agent and the second agent, in general, the arbitrary constitution is accommodated in a separate container from the double structure container.

The aerosol-type hair cosmetic material product may properly include other arbitrary constitution. For example, the aerosol-type hair cosmetic material product may be constituted to include a brush, a comb, a cup for mixing, a pair of gloves, a hair cap, or the like.

### EXAMPLES

Working examples of the second embodiment of the present application are hereunder described. It should be construed that the technical scope of the invention as disclosed in the present application is not limited to the following Examples. It is to be noted that a numerical value expressing each of contents in the tables is a mass % unit.

Second agents of aerosol-type hair cosmetic material compositions according to Examples 1 to 25 and Comparative Examples 1 to 5 of the second embodiment as shown in the following Tables 5 to 9 were prepared. It is to be noted that in each of the respective Examples and respective Comparative Examples 2 and 5, the second agents could be prepared as an emulsion, whereas in Comparative Examples 1, 3, and 4, the second agents could not be emulsified but became a solubilized (transparent) material.

### [Measurement of Average Emulsion Particle Diameter]

After the preparation of each of the second agents according to each of the respective Examples and respective Comparative Examples 2 and 5, its average emulsion particle diameter was measured according to the following “Particle diameter measurement method”. The measurement results are described in the “Emulsion particle diameter” row in the tables.

It is to be noted that even when the second agent according to each of the Examples was filled in a double structure aerosol container in which a space for filling the second agent and a propellant filling space were provided independently of each other and then discharged, the resulting discharged second agent had substantially the same average emulsion particle diameter as that after the preparation as described above.

### <Particle Diameter Measurement Method>

The average emulsion particle diameter was measured by using a laser diffraction scattering method particle size distribution measuring device (a trade name: MICROTRAC MT3000II, available from Nikkiso Co., Ltd.). 10 mL of each second agent (sample) was diluted with water to 100 mL, stirred with a magnetic stirrer at a rotating rate of 1,000 rpm for 10 minutes, and then injected into the device. The measurement was performed twice, and an average was determined. The measurement was performed under the following conditions. Measurement temperature: 25° C., measurement time: 30 seconds, particle refractive index: 1.81, particle shape: non-spherical, solvent: water, and solvent refractive index: 1.33. A volume particle size distribution of the measurement sample was measured. From the measurement results, a median diameter (d50 value) at which an accumulated volume from the small particle diameter side in the accumulated volume distribution became 50% was calculated as an average emulsion particle diameter of particles.

### [Stability Test of Hydrogen Peroxide]

A mass of hydrogen peroxide in the second agent was quantitated by the oxidation-reduction titration method.

After the preparation of the second agent according to each of the Examples or each of the Comparative Examples, first of all, a first measurement of hydrogen peroxide amount was performed.

Subsequently, a first agent was prepared according to the conventional procedure, the first agent and the second agent according to each of the Examples or each of the Comparative Examples were filled in the double structure container shown in FIG. 1, and a product filled with compressed nitrogen (internal pressure: 0.5 MPa) as a propellant was produced. It is to be noted that the outer container and the second inner container are made transparent and light-permeable.

Subsequently, the second agent was subjected to an irradiation treatment with an artificial solar lighting under the following conditions.
Lighting device: Artificial solar lighting, SOLAX XC-500AF Model (manufactured by Seric Ltd.)
Height to subject: Irradiation from a height of 80 cm
Temperature: 25° C.
Irradiation schedule: [(irradiation: 6 hours)+(lights out: 24 hours)]×7 times

After the irradiation treatment with the artificial solar lighting was repeatedly performed, a second measurement of hydrogen peroxide amount was performed.

Subsequently, a residual rate of hydrogen peroxide was calculated according to the following calculation formula.

Residual rate of hydrogen peroxide (%) [Hydrogen peroxide amount at the second measurement]/[Hydrogen peroxide amount at the first measurement]×100 Calculation formula:

The case where the residual rate of hydrogen peroxide is 96% or more and up to 100% is graded as “5”; the case where the residual rate of hydrogen peroxide is 95% or more and less than 96% is graded as “4”; the case where the residual rate of hydrogen peroxide is 90% or more and less than 95% is graded as “3”; the case where the residual rate of hydrogen peroxide is 85% or more and less than 90% is graded as “2”; and the case where the residual rate of hydrogen peroxide is less than 85% is graded as “1”. The test results are described in the “Stability of hydrogen peroxide” row in the tables.

It is to be noted that even after the irradiation treatment with the artificial solar lighting was repeatedly performed, the second agent according to each of the Examples had substantially the same average emulsion particle diameter as that after the preparation as described above.

TABLE 5

| Component | Example 1 | Example 2 | Example 3 | Example 4 | Example 5 | Example 6 |
|---|---|---|---|---|---|---|
| Cetanol | 4 | | | | 2 | 1 |
| Stearyl alcohol | 1 | 4 | 1 | 1 | 1 | 1 |
| Behenyl alcohol | | 1 | | | | |
| Myristyl alcohol | | | | 4 | | |
| Lauryl alcohol | | | 4 | | | |
| Oleyl alcohol | | | | | | |
| 2-Octyldodecanol | | | | | | |
| POE(30) cetyl ether (HLB: 16.9) | 1 | 1 | 1 | 1 | 1 | 1 |
| POE(2) cetyl ether (HLB: 5.4) | 0.5 | 0.5 | 0.5 | 0.5 | 0.5 | 0.5 |
| POE(10) cetyl ether (HLB: 12.9) | | | | | | |
| POE(10) behenyl ether (HLB: 11.5) | | | | | | |
| Stearyltrimethylammonium chloride | 1.5 | 1.5 | 1.5 | 1.5 | 1.5 | 1.5 |
| Lauryltrimethylammonium chloride | | | | | | |
| Sodium laureth sulfate | | | | | | |
| Cocamidopropyl betaine | | | | | | |
| Vaseline | 2 | 2 | 2 | 2 | 2 | 2 |
| Microcrystalline wax | | | | | | |
| Liquid paraffin | | | | | | |
| Cetyl octanoate | | | | | | |
| Stearyl stearate | | | | | | |
| Phenoxyethanol | 0.2 | 0.2 | 0.2 | 0.2 | 0.2 | 0.2 |
| Hydroxyethanediphosphonic acid | 0.15 | 0.15 | 0.15 | 0.15 | 0.15 | 0.15 |
| Tetrasodium | 0.3 | 0.3 | 0.3 | 0.3 | 0.3 | 0.3 |
| hydroxyethanediphosphonate | | | | | | |
| 35% hydrogen peroxide water | 15.7 | 15.7 | 15.7 | 15.7 | 15.7 | 15.7 |
| Phosphoric acid | Adjusted to a pH of 3 | | | | | |
| Purified water | Proper amount | Proper amount | Proper amount | Proper amount | Proper amount | Proper amount |
| Total | 100 | 100 | 100 | 100 | 100 | 100 |
| Emulsion particle diameter (μm) | 14.2 | 14.5 | 4.1 | 4.3 | 18.2 | 25.8 |
| Stability of hydrogen peroxide | 5 | 5 | 5 | 5 | 5 | 4 |

TABLE 6

| Component | Example 7 | Example 8 | Example 9 | Example 10 | Example 11 | Example 12 | Example 13 |
|---|---|---|---|---|---|---|---|
| Cetanol | 4 | 4 | 4 | 4 | 4 | 4 | 4 |
| Stearyl alcohol | | | 1 | 1 | 1 | 1 | 1 |
| Behenyl alcohol | | | | | | | |
| Myristyl alcohol | | | | | | | |
| Lauryl alcohol | | | | | | | |
| Oleyl alcohol | 1 | | | | | | |
| 2-Octyldodecanol | | 1 | | | | | |
| POE(30) cetyl ether (HLB: 16.9) | 1 | 1 | 1 | 1 | 1 | 1 | 1 |
| POE(2) cetyl ether (HLB: 5.4) | 0.5 | 0.5 | 0.5 | 0.5 | 0.5 | 0.5 | 0.5 |
| POE(10) cetyl ether (HLB: 12.9) | | | | | | | |
| POE(10) behenyl ether | | | | | | | |
| (HLB: 11.5) | | | | | | | |
| Stearyltrimethylammonium | 1.5 | 1.5 | | | 1.5 | 1.5 | 1.5 |
| chloride | | | | | | | |
| Lauryltrimethylammonium | | | | | | | |
| chloride | | | | | | | |
| Sodium laureth sulfate | | | 1 | | | | |
| Cocamidopropyl betaine | | | | 3.4 | | | |
| Vaseline | 2 | 2 | 2 | 2 | | | |
| Microcrystalline wax | | | | | 2 | | |
| Liquid paraffin | | | | | | 2 | |
| Cetyl octanoate | | | | | | | 2 |
| Stearyl stearate | | | | | | | |
| Phenoxyethanol | 0.2 | 0.2 | 0.2 | 0.2 | 0.2 | 0.2 | 0.2 |
| Hydroxyethanediphosphonic | 0.15 | 0.15 | 0.15 | 0.15 | 0.15 | 0.15 | 0.15 |
| acid | | | | | | | |
| Tetrasodium | 0.3 | 0.3 | 0.3 | 0.3 | 0.3 | 0.3 | 0.3 |
| hydroxyethanediphosphonate | | | | | | | |
| 35% hydrogen peroxide water | 15.7 | 15.7 | 15.7 | 15.7 | 15.7 | 15.7 | 15.7 |
| Phosphoric acid | Adjusted to a pH of 3 | | | | | | |
| Purified water | Proper amount | Proper amount | Proper amount | Proper amount | Proper amount | Proper amount | Proper amount |
| Total | 100 | 100 | 100 | 100 | 100 | 100 | 100 |
| Emulsion particle diameter (μm) | 15.0 | 15.8 | 3.3 | 34.7 | 15.9 | 10.7 | 17.8 |
| Stability of hydrogen peroxide | 5 | 5 | 5 | 4 | 5 | 5 | 5 |

TABLE 7

| Component | Example 14 | Example 15 | Example 16 | Example 17 | Example 18 | Example 19 |
|---|---|---|---|---|---|---|
| Cetanol | 4 | 4 | 1 | 4 | 4 | 4 |
| Stearyl alcohol | 1 | 1 | | 1 | 1 | 1 |
| Behenyl alcohol | | | | | | |
| Myristyl alcohol | | | | | | |
| Lauryl alcohol | | | | | | |
| Oleyl alcohol | | | | | | |
| 2-Octyldodecanol | | | | | | |
| POE(30) cetyl ether (HLB: 16.9) | 1 | 1 | 0.3 | 3 | 0.5 | |
| POE(2) cetyl ether (HLB: 5.4) | 0.5 | 0.5 | 0.2 | 2 | 1 | 0.5 |
| POE(10) cetyl ether (HLB: 12.9) | | | | | | 1 |
| POE(10) behenyl ether (HLB: 11.5) | | | | | | |
| Stearyltrimethylammonium chloride | 1.5 | 1.5 | 1.5 | 1.5 | 1.5 | 1.5 |
| Lauryltrimethylammonium chloride | | | | | | |
| Sodium laureth sulfate | | | | | | |
| Cocamidopropyl betaine | | | | | | |
| Vaseline | | | 2 | 2 | 2 | 2 |
| Microcrystalline wax | | | | | | |
| Liquid paraffin | | | | | | |
| Cetyl octanoate | | | | | | |
| Stearyl stearate | 2 | | | | | |
| Phenoxyethanol | 0.2 | 0.2 | 0.2 | 0.2 | 0.2 | 0.2 |
| Hydroxyethanediphosphonic acid | 0.15 | 0.15 | 0.15 | 0.15 | 0.15 | 0.15 |
| Tetrasodium | 0.3 | 0.3 | 0.3 | 0.3 | 0.3 | 0.3 |
| hydroxyethanediphosphonate | | | | | | |
| 35% hydrogen peroxide water | 15.7 | 15.7 | 15.7 | 15.7 | 15.7 | 15.7 |
| Phosphoric acid | Adjusted to a pH of 3 | | | | | |
| Purified water | Proper amount | Proper amount | Proper amount | Proper amount | Proper amount | Proper amount |
| Total | 100 | 100 | 100 | 100 | 100 | 100 |
| Emulsion particle diameter (μm) | 18.2 | 15.1 | 25.2 | 7.6 | 6.7 | 12.0 |
| Stability of hydrogen peroxide | 5 | 5 | 4 | 5 | 5 | 5 |

TABLE 8

| Component | Example 20 | Example 21 | Example 22 | Example 23 | Example 24 | Example 25 |
|---|---|---|---|---|---|---|
| Cetanol | 4 | 4 | 0.5 | 0.3 | 0.3 | 0.3 |
| Stearyl alcohol | 1 | 1 | | 0.3 | 0.3 | |
| Behenyl alcohol | | | | | 0.3 | 0.3 |
| Myristyl alcohol | | | | | | |
| Lauryl alcohol | | | | | | 0.3 |
| Oleyl alcohol | | | | | | |
| 2-Octyldodecanol | | | | | | |
| POE(30) cetyl ether (HLB: 16.9) | | | 0.3 | 0.5 | 0.5 | 0.5 |
| POE(2) cetyl ether (HLB: 5.4) | 0.5 | 0.5 | 0.2 | 0.5 | 0.5 | 0.5 |
| POE(10) cetyl ether (HLB: 12.9) | | | | | | |
| POE(10) behenyl ether (HLB: 11.5) | 1 | | | | | |
| Stearyltrimethylammonium chloride | 1.5 | 0.5 | 1.5 | 1.5 | 1.5 | 1.5 |
| Lauryltrimethylammonium chloride | | | | | | |
| Sodium laureth sulfate | | 1 | | | | |
| Cocamidopropyl betaine | | | | | | |
| Vaseline | 2 | 2 | 2 | 2 | 2 | 2 |
| Microcrystalline wax | | | | | | |
| Liquid paraffin | | | | | | |
| Cetyl octanoate | | | | | | |
| Stearyl stearate | | | | | | |
| Phenoxyethanol | 0.2 | 0.2 | 0.2 | 0.2 | 0.2 | 0.2 |
| Hydroxyethanediphosphonic acid | 0.15 | 0.15 | 0.15 | 0.15 | 0.15 | 0.15 |
| Tetrasodium | 0.3 | 0.3 | 0.3 | 0.3 | 0.3 | 0.3 |
| hydroxyethanediphosphonate | | | | | | |
| 35% hydrogen peroxide water | 15.7 | 15.7 | 15.7 | 15.7 | 15.7 | 15.7 |
| Phosphoric acid | Adjusted to a pH of 3 | | | | | |
| Purified water | Proper amount | Proper amount | Proper amount | Proper amount | Proper amount | Proper amount |
| Total | 100 | 100 | 100 | 100 | 100 | 100 |
| Emulsion particle diameter (μm) | 12.5 | 10.0 | 28.1 | 1.9 | 3.8 | 4.5 |
| Stability of hydrogen peroxide | 5 | 5 | 3 | 3 | 3 | 3 |

TABLE 9

| | Comparative | Comparative | Comparative | Comparative | Comparative |
|---|---|---|---|---|---|
| Component | Example 1 | Example 2 | Example 3 | Example 4 | Example 5 |
| Cetanol | | 0.5 | | | |
| Stearyl alcohol | | | | | 0.8 |
| Behenyl alcohol | | | | | |
| Myristyl alcohol | | | | | |
| Lauryl alcohol | | | | | |
| Oleyl alcohol | | | | | |
| 2-Octyldodecanol | | | | | |
| POE(30) cetyl ether (HLB: 16.9) | | 0.3 | | | |
| POE(2) cetyl ether (HLB: 5.4) | | 0.2 | | | 0.2 |
| POE(10) cetyl ether (HLB: 12.9) | | | | | 0.3 |
| POE(10) behenyl ether (HLB: 11.5) | | | | | |
| Stearyltrimethylammonium chloride | | | | | |
| Lauryltrimethylammonium chloride | | | 2.8 | | |
| Sodium laureth sulfate | | 2 | | 3.7 | 2 |
| Cocamidopropyl betaine | 3.5 | | | | |
| Vaseline | | 2 | | | 2 |
| Microcrystalline wax | | | | | |
| Liquid paraffin | | | | | |
| Cetyl octanoate | | | | | |
| Stearyl stearate | | | | | |
| Phenoxyethanol | 0.2 | 0.2 | 0.2 | 0.2 | 0.2 |
| Hydroxyethanediphosphonic acid | 0.15 | 0.15 | 0.15 | 0.15 | 0.15 |
| Tetrasodium hydroxyethanediphosphonate | 0.3 | 0.3 | 0.3 | 0.3 | 0.3 |
| 35% hydrogen peroxide water | 15.7 | 15.7 | 15.7 | 15.7 | 15.7 |
| Phosphoric acid | Adjusted to a pH of 3 | | | | |
| Purified water | Proper amount | Proper amount | Proper amount | Proper amount | Proper amount |
| Total | 100 | 100 | 100 | 100 | 100 |
| Emulsion particle diameter (μm) | Transparent | 0.8 | Transparent | Transparent | 0.7 |
| Stability of hydrogen peroxide | 1 | 2 | 1 | 1 | 2 |

From the results of the foregoing respective tests, it was considered that there is a mutual relation between the average emulsion particle diameter and the stability of hydrogen peroxide. The second agents according to Comparative Examples 1, 3, and 4 were a solubilized material but not an emulsion, and the evaluation thereof regarding the stability of hydrogen peroxide thereof was graded as “1”. In Comparative Examples 2 and 5, the average emulsion particle diameter was less than 1 μm, and the stability of hydrogen peroxide thereof was evaluated to be insufficient.

On the other hand, in the second agent according to each of the Examples, the average emulsion particle diameter was 1 μm or more, and the stability of hydrogen peroxide thereof was evaluated to be good. In Examples 1 to 21 each containing 1% by mass or more of the higher alcohol having 12 to 22 carbon atoms, the stability of hydrogen peroxide thereof was more highly evaluated. In the case where the average emulsion particle diameter is 5 to 20 μm, the evaluation was especially good.

In each of the Examples, while the content of the nonionic surfactant having an HLB value of 12 to 17 was 1% by mass or less, the evaluation thereof regarding the stability of hydrogen peroxide was good.

Even when the second agent according to each of the Examples was filled in the double structure aerosol container, and even when the irradiation treatment with the artificial solar lighting was repeatedly performed, the average emulsion particle diameter was kept. Therefore, it was considered that the average emulsion particle diameter of the second agent was kept even during a period when the second agent was filled in the double structure container in which the residual amount can be visually recognized.

### Third Embodiment

### [Hair Cosmetic Material]

First of all, the hair cosmetic material of the third embodiment of the present invention is explained centering on a first agent and a second agent. Details of main components mentioned in this embodiment are described later.

The hair cosmetic material of the present invention is constituted to include at least a first agent containing an alkali agent and a second agent containing an oxidizing agent. These first agent and second agent are respectively filled in a space for filling the first agent and a space for filling the second agent, each of which is, for example, a bag-like body, in a separate filling/same pressure discharge-type double structure container as described later. Each of the first agent and the second agent of the hair cosmetic material is a liquid dosage form and is discharged as a liquid from the double structure container. Although the contents of the “liquid dosage form” are not always limited, examples thereof include a cream, a gel, a milky lotion, and the like. Of those, a cream and a gel, in which a relatively high viscosity is liable to be ensured, are preferred.

As the hair cosmetic material, such a two-agent type composed of the first agent and the second agent is exemplified; however, a multi-agent type such as a three-agent type, in which a third agent or the like according to an appropriate preparation is further added, is also included. The third agent or the like may be a liquid or may be a powder or the like. In the case where the hair cosmetic material is a three-agent type or the like, in general, the third agent or the like is attached to the double structure container having the first agent and the second agent filled therein, whereby it becomes a constituent element of a hair cosmetic material product as a commodity.

Examples of a category of the hair cosmetic material include an oxidation hair dyeing agent, a hair bleaching agent, and a hair dedyeing agent. Although these are common from the standpoint of including the first agent containing an alkali agent and the second agent containing an oxidizing agent, the oxidation hair dyeing agent further includes an oxidation dye. The oxidation dye is composed of a principal intermediate, or composed of a principal intermediate and a coupler; however, as the case may be, a direct dye is further added. In the hair dedyeing agent, a persulfate is added as an oxidation aid in addition to the alkali agent.

In the hair cosmetic material of the present invention, a viscosity of each of the first agent and the second agent falls within the range of from 7,000 to 30,000 mPa·s at 25° C. and more preferably falls within the range of from 10,000 to 25,000 mPa·s.

This viscosity can be, for example, measured by using a B-type viscometer for one minute at a rotating rate of 12 rpm/min under conditions of using a No. 4 rotor. As a specific example of the B-type viscometer, for example, a BL-type viscometer, VISCOMETER (available from Toki Sangyo Co., Ltd.) can be exemplified.

Each of the first agent and the second agent of the hair cosmetic material may not contain a propellant for foaming, or may contain a propellant for foaming. In the case where each of the first agent and the second agent contains a propellant for foaming, the hair cosmetic material is corresponding to the category of an aerosol-type foam hair cosmetic material. As the propellant for foaming, liquefied gases, such as LPG, dimethyl ether, isopentane, etc., and compressed gases, such as carbon dioxide, a nitrogen gas, etc., can be exemplified; however, in particular, liquefied gases are preferred. Amass ratio of a neat liquid of the first agent or the second agent (a composition in a state of not containing a propellant) to the propellant preferably falls within the range of from 90/10 to 98/2.

Even in the hair cosmetic material not containing a propellant for foaming, the hair cosmetic material may also be a hair cosmetic material in which either one agent of the first agent and the second agent contains an organic acid, for example, citric acid, etc., the other agent contains a carbonate such as sodium carbonate, or a hydrogencarbonate such as sodium hydrogencarbonate, and these agents are mixed to form a foam.

Next, each of the first agent and the second agent of the hair cosmetic material can contain a surfactant. As for the kind of the surfactant, any of a cationic surfactant, an anionic surfactant, an ampholytic surfactant, or a nonionic surfactant may be used, and these may also be arbitrarily combined. However, the case where when the first agent contains an anionic surfactant, then the second agent contains a cationic surfactant; or conversely, when the first agent contains a cationic surfactant, then the second agent contains an anionic surfactant, is preferred from the standpoint of making it easier to achieve uniform mixing after discharging the first agent and the second agent. Although a content of the surfactant in each of the first agent and the second agent is not limited, it is preferably 10% by mass or less in each case, and more preferably in the range of from 2.5 to 8% by mass in each case.

Each of the first agent and the second agent of the hair cosmetic material can contain an oily component. Although a content of the oily component in each of the first agent and the second agent is not limited, it is preferably 10% by mass or less, and more preferably 8% by mass or less in each case. As the oily component, hydrocarbons or esters are especially preferred.

Furthermore, when a relation between the content of the oily component of the first agent (former) and the content of the oily component of the second agent (latter) is allowed to fall within the range of 1.05 to 5, and especially within the range of 1.1 to 3 in terms of a mass % unit of the former to the latter, an oily feeling is different between the former and the latter, and hence, such is preferred from the standpoint of inhibiting the intermixing of the first agent and the second agent, each of which has leaked out into a propellant filling space.

Next, the first agent and/or the second agent of the hair cosmetic material can contain a higher alcohol. A higher alcohol having a carbon number in the range of from 12 to 22 is especially preferred. A preferred content of the higher alcohol to be contained in each of the first agent and the second agent can be mentioned by a higher alcohol index that is an integrated value (a×b) of a carbon number (a) of the higher alcohol and a content value (b) in the first agent or the second agent of the higher alcohol in terms of a mass % unit. That is, a total value of the higher alcohol indexes regarding the higher alcohol contained in each of the first agent and the second agent is preferably 140 or less in each case, and more preferably in the range of from 40 to 130 in each case.

Furthermore, when a relation between a total value of the higher alcohol indexes of the first agent (former) and a total value of the higher alcohol indexes of the second agent (latter) is allowed to fall within the range of 1.05 to 5, and especially within the range of 1.1 to 3 in terms of a mass % unit of the former to the latter, an oily feeling is different between the former and the latter, and hence, such is preferred from the standpoint of inhibiting the intermixing of the first agent and the second agent, each of which has leaked out into a compressed gas filling space.

In addition to the foregoing points, it is also preferred that a higher alcohol having 16 or less carbon atoms accounts for 50% by mass or more of the higher alcohol to be compounded in either one agent of the first agent and the second agents, whereas a higher alcohol having 18 or more carbon atoms accounts for 50% by mass or more of the higher alcohol to be compounded in the other agent. In this case, since an oily feeling is different between the first agent and the second agent, though mixing in such a state that an artificial external force does not act, such as a state of intermixing of the first agent and the second agent, each of which has leaked out into a propellant filling space, hardly occurs, mixing in such a state that an artificial external force acts, such as a state of mixing after discharge of the first agent and the second agent (for example, mixing/application by a brush), is easy.

### [Principal Components of Hair Cosmetic Material]

Next, embodiments of the essential components and the principal arbitrary compounding components to be contained in the hair cosmetic material of the present invention are successively described in detail.

### (Alkali Agent)

In the case where the hair cosmetic material is an oxidation hair dyeing agent, a hair bleaching agent, or a hair dedyeing agent, examples of the alkali agent to be contained in the first agent include ammonia, alkanolamines, silicates, carbonates, hydrogencarbonates, metasilicates, sulfates, chlorides, phosphates, basic amino acids, and the like. Specifically, examples of the alkanolamine include monoethanolamine, triethanolamine, and the like; examples of the silicate include sodium silicate and potassium silicate; examples of the carbonate include sodium carbonate and ammonium carbonate; examples of hydrogencarbonate include sodium hydrogencarbonate and ammonium hydrogencarbonate; examples of the metasilicate include sodium metasilicate and potassium metasilicate; examples of the sulfate include ammonium sulfate; examples of the chloride include ammonium chloride; examples of the phosphate include monobasic ammonium phosphate and dibasic ammonium phosphate; and examples of the basic amino acid include arginine, lysine, and salts thereof. Of these, ammonia, carbonates, and ammonium salts are preferred.

Although a content of the alkali agent in the first agent is not limited, it is, for example, 0.1 to 15% by mass, and more preferably 1 to 10% by mass.

### (Oxidizing Agent and Oxidation Aid)

In the case where the hair cosmetic material is an oxidation hair dyeing agent, a hair bleaching agent, or a hair dedyeing agent, examples of the oxidizing agent to be contained in the second agent include hydrogen peroxide, urea peroxide, melamine peroxide, sodium percarbonate, potassium percarbonate, sodium perborate, potassium perborate, ammonium persulfate, sodium peroxide, potassium peroxide, magnesium peroxide, barium peroxide, calcium peroxide, strontium peroxide, hydrogen peroxide adducts of sulfates, hydrogen peroxide adducts of phosphates, hydrogen peroxide adducts of pyrophosphates, and the like. Of these, hydrogen peroxide is preferred.

Although a content of the oxidizing agent in the second agent is not particularly limited, it is, for example, 0.1 to 15% by mass, and more preferably 1 to 10% by mass. In the case where the second agent contains hydrogen peroxide as the oxidizing agent, it is preferred that ethylene glycol phenyl ether (phenoxyethanol), or hydroxyethanediphosphonic acid or a salt thereof is compounded as a stabilizer for enhancing the stability in the acidic agent.

Meanwhile, examples of the oxidation aid include persulfates, such as ammonium persulfate, potassium persulfate, sodium persulfate, etc.

### (Oxidation Dye and Direct Dye)

In the case where the hair cosmetic material is an oxidation hair dyeing agent, among oxidation dyes to be contained in the first agent, the principal intermediate is a dye precursor that is mainly an o- or p-phenylenediamine or an aminophenol, and in general, it is a compound that is colorless or weakly colored itself. The principal intermediate is used alone, or used together with a coupler.

Examples of the principal intermediate include p-phenylenediamine, toluene-2,5-diamine (p-toluylene-diamine), N-phenyl-p-phenylenediamine, 4,4′-diamino-diphenylamine, p-aminophenol, o-aminophenol, p-methylaminophenol, N,N-bis(2-hydroxyethyl)-p-phenylene-diamine, 2-hydroxyethyl-p-phenylenediamine, o-chloro-p-phenylenediamine, 4-amino-m-cresol, 2-amino-4-hydroxyethylaminoanisole, 2,4-diaminophenol, and salts thereof, and the like. Examples of the salt include hydrochlorides, sulfates, acetates, and the like.

As the coupler, m-diamines, m-aminophenols, and m-diphenols are mainly exemplified. Specifically, examples thereof include resorcin, catechol, pyrogallol, phloroglucin, gallic acid, hydroquinone, 5-amino-o-cresol, m-aminophenol, 5-(2-hydroxyethylamino)-2-methylphenol, m-phenylenediamine, 2,4-diaminophenoxyethanol, toluene-3,4-diamine, α-naphthol, 2,6-diaminopyridine, diphenylamine, 3,3′-iminodiphenyl, 1,5-dihydroxynaphthalene, tannic acid, and salts thereof, and the like.

Examples of the direct dye which may be additionally used for the purpose of regulating the dyed hair color tone include various acid dyes, basic dyes, nitro dyes, natural dyes, disperse dyes, and HC dyes, and the like.

### (Surfactant)

A surfactant can be contained in the first agent and/or the second agent of the hair cosmetic material. Various cationic, anionic, ampholytic or nonionic surfactants can be used as the surfactant. In all of the first agent and the second agent, a content of the surfactant is preferably 10% by mass or less in each case, and especially preferably in the range of from 2.5 to 8% by mass in each case.

Examples of the cationic surfactant include lauryltrimethylammonium chloride, cetyltrimethylammonium chloride, stearyltrimethylammonium chloride (steartrimonium chloride), behenyltrimethylammonium chloride (behentrimonium chloride), distearyldimethylammonium chloride, cetyltrimethylammonium bromide, stearyltrimethylammonium bromide, an ethyl sulfuric acid lanolin fatty acid aminopropylethyl dimethylammonium, stearyltrimethylammonium saccharinate, cetyltrimethylammonium saccharinate, methacryloyloxyethyltrimethylammonium chloride, behenyltrimethylammonium methyl sulfate, and the like.

Examples of the anionic surfactant include alkyl ether sulfates, polyoxyethylene (hereinafter referred to as “POE”) alkyl ether sulfates, alkyl sulfates, alkenyl ether sulfates, alkenyl sulfates, olefin sulfonates, alkane sulfonates, saturated or unsaturated fatty acid salts, alkyl or alkenyl ether carboxylates, α-sulfone fatty acid salts, N-acylamino acid type surfactants, phosphoric mono- or diester type surfactants, and sulfosuccinic acid esters. A counter ion of an anionic group of such a surfactant may be any of a sodium ion, a potassium ion, or triethanolamine.

More specifically, examples of the anionic surfactant include sodium lauryl sulfate, sodium myristyl sulfate, potassium lauryl sulfate, ammonium lauryl sulfate, triethanolamine lauryl sulfate, sodium cetyl sulfate, sodium stearyl sulfate, polyoxyethylene (POE) lauryl ether sodium sulfate, POE lauryl ether triethanolamine sulfate, POE lauryl ether ammonium sulfate, POE stearyl ether sodium sulfate, sodium stearoylmethyltaurate, triethanolamine dodecylbenzenesulfonate, sodium tetradecenesulfonate, sodium lauryl phosphate, POE lauryl ether phosphoric acid and salts thereof, N-lauroyl glutamates (e.g., sodium lauroyl glutamate, etc.), N-lauroylmethyl-β-alanine salts, N-acyl glycine salts, and N-acyl glutamates, as well as lauric acid and myristic acid, each of which is a higher fatty acid, and salts of these higher fatty acids.

Examples of the ampholytic surfactant include alkyl betaine types, fatty acid amide propyl betaine types, alkyl imidazole types, and amino acid types.

More specifically, examples of the ampholytic surfactant include lauryl betaine, imidazoline, amide betaine, carbobetaine, sulfobetaine, hydroxysulfobetaine, amide sulfobetaine, sodium 2-undecyl-N-carboxymethyl-N-hydroxy-ethyl imidazolinium betaine, cocoamidopropyl betaine, lauryl dimethylaminoacetic acid betaine, stearyl dimethylaminoacetic acid betaine, coconut oil fatty acid amidopropyl betaine, and the like.

Examples of the nonionic surfactant include ether types and ester types.

Specifically, examples of the ether-type nonionic surfactant may include POE cetyl ether (ceteth), POE stearyl ether (steareth), POE behenyl ether, POE oleyl ether (oreth), POE lauryl ether (laureth), POE octyl dodecyl ether, POE hexyl decyl ether, POE isostearyl ether, POE nonyl phenyl ether, and POE octyl phenyl ether.

Specifically, examples of the ester-type nonionic surfactant may include monooleic acid POE sorbitan, monostearic acid POE sorbitan, monopalmitic acid POE sorbitan, monolauric acid POE sorbitan, trioleic acid POE sorbitan, monostearic acid POE glycerin, monomyristic acid POE glycerin, tetraoleic acid POE sorbite, hexastearic acid POE sorbite, monolauric acid POE sorbite, POE sorbite beeswax, monooleic acid polyethylene glycol, monostearic acid polyethylene glycol, monolauric acid polyethylene glycol, lipophilic glyceryl monooleate, lipophilic glyceryl monostearate, self-emulsifying glyceryl monostearate, sorbitan monooleate, sorbitan sesquioleate, sorbitan trioleate, sorbitan monostearate, sorbitan monopalmitate, sorbitan monolaurate, sucrose fatty acid ester, decaglyceryl monolaurate, decaglyceryl monostearate, decaglyceryl monooleate, and decaglyceryl monomyristate.

### (Oily Component)

An oily component can be contained in the first agent and/or the second agent of the hair cosmetic material. Examples of the oily component include a fat and oil, a wax, a higher fatty acid, an alkyl glyceryl ether, an ester, a silicone, a hydrocarbon, and the like. In all of the first agent and the second agent, a content of the oily component is preferably 10% by mass or less, and more preferably 8% by mass or less in each case.

Examples of the fat and oil include olive oil, rose hip oil, camellia oil, shea butter, macadamia nut oil, almond oil, tea seed oil, safflower oil, sunflower oil, soybean oil, cottonseed oil, sesame oil, beef tallow, cacao butter, corn oil, peanut oil, rapeseed oil, rice bran oil, rice germ oil, wheat germ oil, Coix lacryma-jobi seed oil, grape seed oil, avocado oil, carrot oil, castor oil, linseed oil, coconut oil, mink oil, egg yolk oil, and the like.

Examples of the wax include beeswax, candelilla wax, carnauba wax, jojoba oil, lanolin, spermaceti wax, rice bran wax, sugar cane wax, palm wax, montan wax, cotton wax, bayberry wax, shellac wax, and the like.

Examples of the higher fatty acid include lauric acid, myristic acid, palmitic acid, stearic acid, behenic acid, isostearic acid, hydroxystearic acid, 12-hydroxystearic acid, oleic acid, undecylenic acid, linoleic acid, ricinoleic acid, lanolin fatty acid, and the like.

Examples of the alkyl glyceryl ether include batyl alcohol (monostearyl glyceryl ether), chimyl alcohol (monocetyl glyceryl ether), selachyl alcohol (monooleyl glyceryl ether), isostearyl glyceryl ether, and the like.

Examples of the ester include diisobutyl adipate, cetyl octanoate, isononyl isononanoate, diisopropyl sebacate, octyldodecyl myristate, isopropyl palmitate, stearyl stearate, hexyl laurate, hexyldecyl dimethyloctanoate, triisodecyl myristate, fatty acids (C10-30) (cholesteryl/lanosteryl), lauryl lactate, acetylated lanolin, ethylene glycol di-2-ethylhexanoate, dipentaerythritol fatty acid esters, N-alkyl glycol monoisostearates, diisostearyl malate, and the like.

Examples of the silicone include dimethyl polysiloxane (INCI name: dimethicone), dimethyl polysiloxane having a hydroxyl terminal group (INCI name: dimethiconol), methylphenyl polysiloxane, decamethyl cyclopentasiloxane, a polyether-modified silicone, a highly polymerized silicone having an average polymerization degree of 650 to 10,000, an amino-modified silicone, a betaine-modified silicone, an alkyl-modified silicone, an alkoxy-modified silicone, a carboxy-modified silicone, and the like.

Among the foregoing, examples of the amino-modified silicone include an aminopropylmethylsiloxane-dimethyl-siloxane copolymer (INCI name: aminopropyl dimethicone), an aminoethylaminopropylsiloxane-dimethylsiloxane copolymer (INCI name: amodimethicone), an aminoethylaminopropyl-methylsiloxane-dimethylsiloxane copolymer (INCI name: trimethylsilylamodimethicone), and the like.

Examples of the hydrocarbon include an α-olefin oligomer, a light isoparaffin, a light liquid isoparaffin, a liquid isoparaffin, a liquid paraffin, squalane, polybutene, a paraffin, microcrystalline wax, vaseline, and the like.

### (Higher Alcohol)

The specified higher alcohol can be contained in the first agent and/or the second agent of the hair cosmetic material. The higher alcohol as referred to herein refers to a monohydric alcohol having 12 or more and 22 or less carbon atoms, which is a linear or branched, saturated or unsaturated alcohol.

A content of the higher alcohol in each of the first agent and the second agent is not always limited. However, when a preferred content thereof is mentioned in terms of the above-described “higher alcohol index”, a total value of the higher alcohol indexes, that is an integrated value (a×b) of a carbon number (a) of the higher alcohol and a content value (b) in the first agent or the second agent of the higher alcohol in terms of a mass % unit, is preferably 140 or less, and especially preferably 130 or less in all of the first agent and the second agent.

Specifically, examples of the linear, saturated higher alcohol may include lauryl alcohol, myristyl alcohol, cetyl alcohol (cetanol), stearyl alcohol, arachyl alcohol, and behenyl alcohol. Besides, examples of the branched, saturated higher alcohol may include isostearyl alcohol, 2-hexyldodecanol, 2-octyldodecanol, and the like, and examples of the unsaturated higher alcohol may include oleyl alcohol and the like.

Among the foregoing, lauryl alcohol, myristyl alcohol, cetyl alcohol (cetanol), stearyl alcohol, arachyl alcohol, and behenyl alcohol, all of which are a linear, saturated higher alcohol having a carbon number falling within the range of from 12 to 22, are especially preferred.

### [Other Arbitrary Compounding Components of Hair Cosmetic Material]

In the first agent and/or the second agent of the hair cosmetic material, in addition to the above-described various components, for example, a cationic polymer, a solubilizing agent, a water-soluble polymer compound, a saccharide, an antiseptic, a stabilizer, a pH adjuster, a plant extract, a crude drug extract, a vitamin, a perfume, an antioxidant, an ultraviolet light absorber, a chelating agent, or the like can be arbitrarily compounded. Some of them are hereunder specifically described.

### (Cationic Polymer)

Examples of the cationic polymer include cationized cellulose derivatives, polymers or copolymers of diallyl quaternary ammonium salts, and quaternized polyvinylpyrrolidone, and besides, cationic starches, cationized guar gum, and the like.

Examples of the cationized cellulose derivative include a polymer of a quaternary ammonium salt, which is obtained by adding glycidyltrimethylammonium chloride to hydroxyethyl cellulose (polyquaternium-10, for example, LEOGUARD G and LEOGUARD GP, all of which are available from Lion Corporation; and POLYMER JR-125, POLYMER JR-400, POLYMER JR-30M, POLYMER LR-400, and POLYMER LR-30M, all of which are available from Amercho), a hydroxyethyl cellulose/dimethyldiallylammonium chloride copolymer (polyquaternium-4, for example, CELQUAT H-100 and CELQUAT L-200, all of which are available from National Starch and Chemical Corporation), and the like.

Examples of the polymer or copolymer of a diallyl quaternary ammonium salt include a dimethyldiallylammonium chloride polymer (polydimethylmethylene piperidinium chloride) [polyquaternium-6, for example, MERQUAT 100, available from The Lubrizol Corporation], a dimethyldiallylammonium chloride/acrylic acid copolymer [polyquaternium-22, for example, MERQUAT 280, available from The Lubrizol Corporation], an acrylic acid/dially quaternary ammonium salt/acrylamide copolymer [polyquaternium-39, for example, MERQUAT PLUS 3331, available from The Lubrizol Corporation], and the like.

Examples of the quaternized polyvinylpyrrolidone include a quaternary ammonium salt obtained from a copolymer of vinylpyrrolidone (VP) and dimethylaminoethyl methacrylate and diethyl sulfate [polyquaternium-11, for example, GAFQUAT 734 and GAFQUAT 755, all of which are available from ISP Japan Ltd.] and the like.

### (Solubilizing Agent)

The solubilizing agent is compounded for the purpose of rending each of the agents of the hair cosmetic material composition liquid. Examples of the solubilizing agent include water, polyhydric alcohols, and organic solvents. Examples of the polyhydric alcohol include glycols and glycerins. Examples of the glycol include ethylene glycol, diethylene glycol, triethylene glycol, propylene glycol, dipropylene glycol, isoprene glycol, 1,3-butylene glycol, and the like; and examples of the glycerin include glycerin, diglycerin, polyglycerin, and the like. Examples of the organic solvent include ethanol, n-propanol, isopropanol, methyl cellosolve, methyl carbitol, benzyl alcohol, phenethyl alcohol, y-phenylpropyl alcohol, cinnamic alcohol, p-methylbenzyl alcohol, α-phenylethanol, phenoxyethanol, phenoxyisopropanol, an N-alkylpyrrolidone, an alkylene carbonate, an alkyl ether, and the like. Water is especially preferably used.

### (Water-Soluble Polymer Compound)

As the water-soluble polymer compound, anionic, nonionic, or ampholytic polymer compounds, exclusive of the above-described cationic polymers can be used. Examples thereof include a carboxyvinyl polymer, a diallyl quaternary ammonium salt/acrylic acid copolymer, and the like.

### [Double Structure Container and Hair Cosmetic Material Product]

### (Double Structure Container)

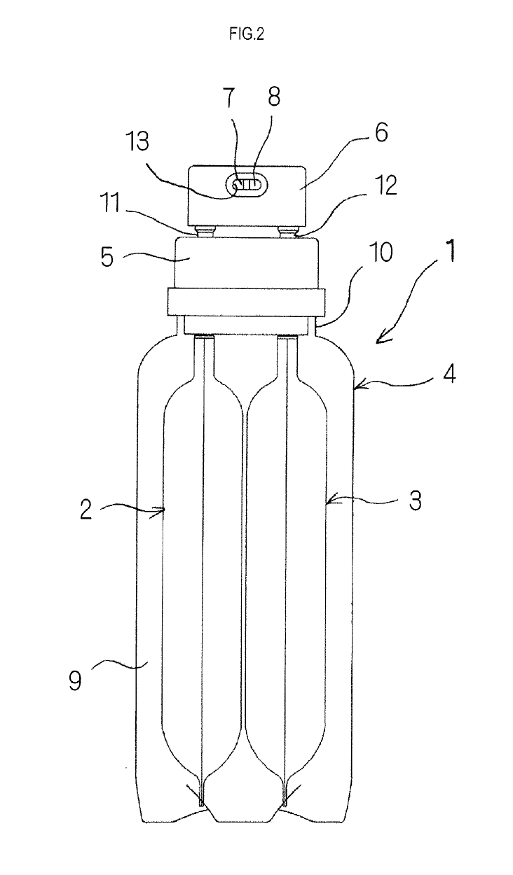

Next, an example of the double structure container which is used in the third embodiment is explained by reference to FIG. 2. In this explanation, portions not related directly to the gist of the present invention are functionally simply explained, and detailed structural explanations thereof are omitted.

An outer container 4 of a double structure container 1 is a pressure-resistant container having such a shape that it is able to stand alone as it is, or by taking a cap (illustration omitted) to be put on a lid as described later as the bottom, in an inverted state and being made of a hard and strong material. Although the outer container 4 may be formed of an opaque metal material, such as stainless steel, etc., it is preferably formed of a plastic material that is hard, strong, and transparent in such a manner that the inside thereof can be seen, and is provided with a needed thickness.

In the inside of the outer container 4, a pouch-shaped first inner container 2 constituting a space for filling the first agent, and a pouch-shaped second inner container 3 constituting a space for filling the second agent are each independently provided. In FIG. 2, on the assumption that the outer container 4 is made of a transparent plastic material, the state in which the pouch-shaped inner containers 2 and 3 in the inside are seen from the outside is illustrated.

The inner containers 2 and 3 are each constituted by using a plastic material different from a constituent material of the outer container 4, the plastic material being comparatively soft so that it is easily deformable by pressure and also being relatively thin and soft. The inner containers 2 and 3 may be each formed in a bag-like body having a laminate structure in consideration of the resistance to breakage. In particular, it is not limited but preferred that the first inner container 2 for filling the first agent containing an alkali agent is made in a laminate structure including a metal layer. Meanwhile, it is not limited but preferred that the second inner container 3 is made transparent or translucent such that the reduced state of the contents in the inner container can be visually recognized.

In the inside of the outer container 4, a space excluding spaces for placing the inner containers 2 and 3 is made as a propellant filling space 9, and a propellant is filled in this space. The propellant is preferably a compressed gas using a nitrogen gas (N₂), carbon dioxide (CO₂), or the like, each of which is inert and low in toxicity, LPG that is a liquefied gas, or the like.

An opening 10 of an upper end of the outer container 4 is airtightly closed by a valve unit 5 that is also a lid. In the inside of the valve unit 5, while illustration is omitted, discharge passages for the first agent and the second agent and valves for closing these discharge passages, respectively are provided. The discharge passages for the first agent and the second agent are connected in a liquid-tight manner to openings of the upper ends of the inner containers 2 and 3, respectively.

It is to be noted that in each of the inner containers 2 and 3, in order to accelerate smooth discharge of the first agent and the second agent to be filled therein, respectively, a rod-like body having a ladder-shaped structure as a whole (illustration omitted; for example, see dip tubes 16A and 16B shown in FIG. 1 of PTL 3) may be inserted from the upper end opening.

As a pair of the valves for opening and closing the discharge passage, a so-called valve stem is adopted in the present embodiment, a pair of cylindrical stems 11 and 12 is protruded in an upper portion of the valve unit 5 and connected in a liquid-tight manner to a pair of discharge passages (illustration omitted) provided in the inside of an actuator 6. These discharge passages in the inside of the actuator 6 are communicated with a pair of discharge holes 7 and 8 provided in an opening 13 of the actuator 6. It is to be noted that the pair of the discharge passages in the inside of the actuator 6 may also be constituted in such a manner that the pair of the discharge passage go into single discharge passage before they reach the opening 13 and are discharged from a single discharge hole of the opening 13.

Meanwhile, the pair of the cylindrical stems 11 and 12 exists at an illustrated position in such a state that it is always pushed upward by a pushing spring (illustration omitted), such as a coil spring built in the valve unit 5, etc., and at this time, a stem valve is in a “closed” state.

### (Hair Cosmetic Material Product)

The hair cosmetic material product of the present embodiment is one in which the first agent and the second agent of the hair cosmetic material are respectively filled in the inner container 2 and the inner container 3 in the double structure container 1. As the case may be, a third agent is attached as an additional constituent element of the hair cosmetic material product.

In the double structure container 1 having the first agent and the second agent filled therein, both of the first agent and the second agent in the inner containers 2 and 3 always receive a discharge pressure by the propellant for pressurization in the propellant filling space 9. Then, when the actuator 6 is subjected to press-down resisting to a pushing force of the pushing spring, the valve stem becomes in an “open” state, whereby the first agent and the second agent are simultaneously discharged. When a pressing force against the actuator 6 is released, the valve stem becomes in a “closed” state, whereby the discharges of the first agent and the second agent are simultaneously stopped.

### EXAMPLES

Next, Examples and Comparative Examples of the third embodiment of the present invention are explained. It should be construed that the technical scope of the present invention is not limited by the following Examples and Comparative Examples.

### [Preparation of Hair Cosmetic Material]

A first agent and a second agent of each of two-agent type oxidation hair dyeing agents according to Examples 1 to 37 and Comparative Examples 1 to 4 in the third embodiment, each having a composition shown in the following Tables 10 to 13, respectively, were prepared according to the conventional procedure. All of these first and second agents are in a cream state. In the tables, the numerical value showing the content of each component is a numerical value in terms of amass % unit in the first agent or the second agent.

Next, the terms “First agent: viscosity” and “Second agent: viscosity” in each of the tables are each a viscosity value (mPa·s) as measured in such a manner that the first agent or the second agent according to each of the Examples or each of the Comparative Examples was stabilized by allowing to stand for 3 days after the preparation, and thereafter, the viscosity was measured by using a BL-type viscometer, VISCOMETER that is a B-type viscometer available from Toki Sangyo Co., Ltd. under measurement conditions of 25° C. using a No. 4 rotor for one minute at a rotating rate of 12 rpm/min.

In addition, the terms “First agent: surfactant amount” and “Second agent: surfactant amount” in each of the tables express each a total content (% by mass) of various surfactants in the first agent or the second agent according to each of the Examples and each of the Comparative Examples.

In addition, the terms “First agent: oil amount” and “Second agent: oil amount” in each of the tables each express a total content (% by mass) of various oily components in the first agent or the second agent according to each of the Examples or each of the Comparative Examples.

In addition, the terms “First agent: (carbon number)×(mass)” and “Second agent: (carbon number)×(mass)” in each of the tables each express a total value of the above-described “higher alcohol indexes” in the first agent or the second agent according to each of the Examples or each of the Comparative Examples.

Although not expressed in the tables, all of the second agents according to the respective Examples and Comparative Examples are adjusted to a pH of 3.8.

### [Evaluation of Hair Cosmetic Material]

The hair cosmetic materials according to the respective Examples and respective Comparative Examples were evaluated in the following manner.

### (Degree of Consumption of Hydrogen Peroxide)

A degree of consumption of hydrogen peroxide in the second agent in a state where the second agent of the hair cosmetic material came into contact with the first agent was evaluated by a change of concentration of hydrogen peroxide before and after the contact with the first agent. This degree of consumption hydrogen peroxide is an index of evaluating a generation amount of an oxygen gas in the contact state between the first agent and the second agent.

That is, in a 100-mL tall beaker (available from Hario Co., Ltd., barrel outer diameter: 50 mm, height: 80 mm), 50 g of the first agent immediately after the preparation according to each of the Examples or each of the Comparative Examples was gently poured, and subsequently, 50 g of the second agent immediately after the preparation according to each of the same Examples or each of the same Comparative Examples was gently poured thereonto. At this time, in all of the Examples and Comparative Examples, a two-layer structure including the first agent in a bottom layer and the second agent in a surface layer was formed in the beaker.

Then, for the purpose of avoiding the generation of an error in the measurement concentration to be caused due to evaporation or volatilization of moisture or a volatile component, an upper end opening of the beaker according to each of the Examples or each of the Comparative Examples was immediately hermetically sealed by SARAN WRAP (a registered trademark) and allowed to gentry stand as it was in a cool, dark place for 24 hours. Subsequently, 20 g of the second agent was collected from a portion in a depth of up to 15 mm from the surface in the surface layer (second agent) in the two-layer structure in the beaker according to each of the Examples or each of the Comparative Examples, and after well stirring this, a hydrogen peroxide concentration D1 (%) was measured.

Meanwhile, a hydrogen peroxide concentration D2 (%) in the second agent just before allowing to stand for 24 hours can be accurately determined by calculation because a predetermined amount (% by mass) of hydrogen peroxide is compounded as 35% hydrogen peroxide water in the second agent, and the second agent immediately after the preparation is used. From the foregoing standpoints, the degree of consumption of hydrogen peroxide in the second agent in the contact state of the second agent with the first agent in the hair cosmetic material was calculated as a change of concentration of hydrogen peroxide before and after the contact with the first agent in terms of a subtracted value (%) of (D2−D1).

The foregoing measurement and calculation of the change of concentration of hydrogen peroxide were performed three times with respect to each of the Examples or each of the Comparative Examples, and an average value thereof was evaluated as the degree of consumption of hydrogen peroxide in the instant Example or Comparative Example. As for evaluation criteria, the case where the subtracted value of (D2−D1) was 3% or less was evaluated as “⊙”; the case where the subtracted value was more than 3% and 6% or less was evaluated as “◯”; the case where the subtracted value was more than 6% and 10% or less was evaluated as “Δ”; and the case where the subtracted value was more than 10% was evaluated as “×”. The evaluation results are described in the “Degree of consumption of hydrogen peroxide” row in each of the tables.

### (Uniform Mixing Properties After Discharge)

In preparing the first agent and the second agent according to each of the Examples or each of the Comparative Examples as described above, a coloring agent was previously added in the first agent, and the first agent in a cream state after the preparation was mixed with the second agent in a cream state. As for this mixing operation, the same operation of mixing by stirring with a brush 15 times at the same speed so as to draw a circle was performed commonly in each of the Examples or each of the Comparative Examples. This mixing operation conforms to the usual uniform mixing of the first agent and the second agent.

The presence or absence of color unevenness of the hair cosmetic material according to each of the Examples or each of the Comparative Examples after the above-described mixing operation was evaluated by 10 panelists. As for evaluation criteria, the case where the color unevenness was not observed at all was evaluated as “⊙”; the case where the color unevenness was not substantially observed was evaluated as “◯”; the case where the color unevenness was somewhat observed was evaluated as “Δ”; and the case where the color unevenness was significantly observed was evaluated as “×”. Then, with respect to the respective Examples and the respective Comparative Examples, the evaluation made by the largest number of the ten panelists was adopted. In the case where there were two or more evaluations made by the largest number of the ten panelists, the lower evaluation was adopted. The evaluation results are described in the “Uniform mixing properties after discharge” row in each of the tables.

### (Brightness)

After preparing the first agent and the second agent in a cream state according to each of the Examples or each of the Comparative Examples, a hair dyeing treatment was performed by uniformly mixing the both agents by using a brush and uniformly applying 2 mL of the mixture to a black hair bundle sample for evaluation having a length of 10 cm, followed by allowing the resultant to stand for 30 minutes. Thereafter, the hair bundle sample was washed with water, dried, and then evaluated for the brightness of hair dyeing by 10 panelists. As for evaluation criteria, the case where the brightness was very good was evaluated as “⊙”; the case where the brightness was good was evaluated as “◯”; the case where the brightness was not bad but could not be said to be good was evaluated as “Δ”; and the case where the brightness was bad was evaluated as “×”.

In all of the cases, the evaluation made by the largest number of the ten panelists was adopted. In the case where there were two or more evaluations made by the largest number of the ten panelists the lower evaluation was adopted. The evaluation results are described in the “Brightness” row in each of the tables.

TABLE 10

| | Example 1 | Example 2 | Example 3 | Example 4 | Example 5 | Example 6 | Example 7 | Example 8 | Example 9 | Example 10 | Example 11 | Example 12 | Example 13 |
|---|---|---|---|---|---|---|---|---|---|---|---|---|---|
| First agent | | | | | | | | | | | | | |
| Behenyl alcohol | | 3 | | 4 | | | | | | | | | |
| Arachyl alcohol | | | 3 | | 4 | | | | | | | | |
| Stearyl stearate | 3 | | | 3 | 3 | 3 | 3 | 3 | 3 | 3 | 3 | 3 | 3 |
| Cetyl alcohol | 4 | 4 | 4 | | | 4 | 4 | 4 | 4 | 4 | 4 | 4 | 4 |
| POE(30) cetyl ether | 2 | 2 | 2 | 2 | 2 | | 2 | 2 | 2 | 2 | | 2 | 2 |
| POE(20) stearyl ether | | | | | | 2 | | | | | | | |
| POE(2) cetyl ether | 1 | 1 | 1 | 1 | 1 | 1 | | 1 | 1 | | 1 | 1 | 1 |
| POE(2) lauryl ether | | | | | | | 1 | | | | | | |
| Glyceryl stearate | | | | | | | | | | 1 | | | |
| Alkyl glucoside | | | | | | | | | | | 2 | | |
| Stearyltrimethylammonium | 1 | 1 | 1 | 1 | 1 | 1 | 1 | 1 | 1 | 1 | 1 | 1 | 1 |
| chloride | | | | | | | | | | | | | |
| Sodium laureth sulfate | | | | | | | | | | | | | |
| Cocamidopropyl betaine | | | | | | | | | | | | | |
| Sodium chloride | | | | | | | | | | | | | |
| Hydroxyethyl cellulose | | | | | | | | | | | | | |
| Vaseline | 3 | 3 | 3 | 3 | 3 | 3 | 3 | | | 3 | 3 | 3 | 3 |
| Cetyl octanoate | | | | | | | | 1 | | | | | |
| Lanolin | | | | | | | | | 2 | | | | |
| Stearyl stearate | | | | | | | | | 1 | | | | |
| Microcrystalline wax | | | | | | | | 2 | | | | | |
| Sodium sulfite | 0.1 | 0.1 | 0.1 | 0.1 | 0.1 | 0.1 | 0.1 | 0.1 | 0.1 | 0.1 | 0.1 | 0.1 | 0.1 |
| p-Phenylenediamine | 0.5 | 0.5 | 0.5 | 0.5 | 0.5 | 0.5 | 0.5 | 0.5 | 0.5 | 0.5 | 0.5 | 0.5 | 0.5 |
| m-Aminophenol | 0.4 | 0.4 | 0.4 | 0.4 | 0.4 | 0.4 | 0.4 | 0.4 | 0.4 | 0.4 | 0.4 | 0.4 | 0.4 |
| Resorcin | 0.5 | 0.5 | 0.5 | 0.5 | 0.5 | 0.5 | 0.5 | 0.5 | 0.5 | 0.5 | 0.5 | 0.5 | 0.5 |
| Ascorbic acid | 0.2 | 0.2 | 0.2 | 0.2 | 0.2 | 0.2 | 0.2 | 0.2 | 0.2 | 0.2 | 0.2 | 0.2 | 0.2 |
| 28% ammonia water | 4 | 4 | 4 | 4 | 4 | 4 | 4 | 4 | 4 | 4 | 4 | 4 | 4 |
| Purified water | Proper | Proper | Proper | Proper | Proper | Proper | Proper | Proper amount | Proper amount | Proper amount | Proper amount | Proper amount | Proper amount |
| | amount | amount | amount | amount | amount | amount | amount | | | | | | |
| Total | 100 | 100 | 100 | 100 | 100 | 100 | 100 | 100 | 100 | 100 | 100 | 100 | 100 |
| Second agent | | | | | | | | | | | | | |
| Stearyl alcohol | 1 | 1 | 1 | 1 | 1 | 1 | 1 | 1 | 1 | 1 | 1 | 1 | 1 |
| Cetyl alcohol | 4 | 4 | 4 | 4 | 4 | 4 | 4 | 4 | 4 | 4 | 4 | 4 | 4 |
| POE(30) cetyl ether | 1 | 1 | 1 | 1 | 1 | 1 | 1 | 1 | 1 | 1 | 1 | 1 | |
| POE(2) cetyl ether | 0.5 | 0.5 | 0.5 | 0.5 | 0.5 | 0.5 | 0.5 | 0.5 | 0.5 | 0.5 | 0.5 | | 0.5 |
| Glyceryl stearate | | | | | | | | | | | | 0.5 | |
| Alkyl glucoside | | | | | | | | | | | | | 1 |
| Stearyltrimethylammonium | 1 | 1 | 1 | 1 | 1 | 1 | 1 | 1 | 1 | 1 | 1 | 1 | 1 |
| chloride | | | | | | | | | | | | | |
| Sodium chloride | | | | | | | | | | | | | |
| Hydroxyethyl cellulose | | | | | | | | | | | | | |
| Vaseline | 2 | 2 | 2 | 2 | 2 | 2 | 2 | 2 | 2 | 2 | 2 | 2 | 2 |
| Microcrystalline wax | | | | | | | | | | | | | |
| Phenoxyethanol | 0.2 | 0.2 | 0.2 | 0.2 | 0.2 | 0.2 | 0.2 | 0.2 | 0.2 | 0.2 | 0.2 | 0.2 | 0.2 |
| Hydroxyethanediphosphonic | 0.1 | 0.1 | 0.1 | 0.1 | 0.1 | 0.1 | 0.1 | 0.1 | 0.1 | 0.1 | 0.1 | 0.1 | 0.1 |
| acid | | | | | | | | | | | | | |
| Tetrasodium | 0.1 | 0.1 | 0.1 | 0.1 | 0.1 | 0.1 | 0.1 | 0.1 | 0.1 | 0.1 | 0.1 | 0.1 | 0.1 |
| hydroxyethanediphosphonate | | | | | | | | | | | | | |
| 35% hydrogen peroxide | 16 | 16 | 16 | 16 | 16 | 16 | 16 | 16 | 16 | 16 | 16 | 16 | 16 |
| Purified water | Proper | Proper | Proper | Proper | Proper | Proper | Proper | Proper amount | Proper amount | Proper amount | Proper amount | Proper amount | Proper amount |
| | amount | amount | amount | amount | amount | amount | amount | | | | | | |
| Total | 100 | 100 | 100 | 100 | 100 | 100 | 100 | 100 | 100 | 100 | 100 | 100 | 100 |
| First agent: viscosity | 18580 | 13640 | 11210 | 20870 | 10720 | 16320 | 19850 | 20100 | 16890 | 27410 | 7100 | 18580 | 7100 |
| Second agent: viscosity | 12650 | 12650 | 12650 | 12650 | 12650 | 12650 | 12650 | 12650 | 12650 | 12650 | 12650 | 19490 | 12650 |
| First agent: surfactant amount | 4 | 4 | 4 | 4 | 4 | 4 | 4 | 4 | 4 | 4 | 4 | 4 | 4 |
| Second agent: surfactant | 2.5 | 2.5 | 2.5 | 2.5 | 2.5 | 2.5 | 2.5 | 2.5 | 2.5 | 2.5 | 2.5 | 2.5 | 2.5 |
| amount | | | | | | | | | | | | | |
| First agent: oil amount | 3 | 3 | 3 | 3 | 3 | 3 | 3 | 3 | 3 | 3 | 3 | 3 | 3 |
| Second agent: oil amount | 2 | 2 | 2 | 2 | 2 | 2 | 2 | 2 | 2 | 2 | 2 | 2 | 2 |
| First agent: | 118 | 130 | 124 | 142 | 134 | 118 | 118 | 118 | 118 | 118 | 118 | 118 | 118 |
| (carbon number) × (mass) | | | | | | | | | | | | | |
| Second agent: | 82 | 82 | 82 | 82 | 82 | 82 | 82 | 82 | 82 | 82 | 82 | 82 | 82 |
| (carbon number) × (mass) | | | | | | | | | | | | | |
| Degree of consumption of | ⊚ | ⊚ | ⊚ | ⊚ | ⊚ | ⊚ | ⊚ | ⊚ | ⊚ | ⊚ | ⊚ | ⊚ | ⊚ |
| hydrogen peroxide | | | | | | | | | | | | | |
| Uniform mixing properties | ⊚ | ⊚ | ⊚ | ⊚ | ⊚ | ⊚ | ⊚ | ⊚ | ⊚ | ⊚ | ⊚ | ⊚ | ⊚ |
| after discharge | | | | | | | | | | | | | |
| Brightness | ⊚ | ⊚ | ⊚ | ⊚ | ⊚ | ⊚ | ⊚ | ⊚ | ⊚ | ⊚ | ⊚ | ⊚ | ⊚ |

TABLE 11

| | Example 14 | Example 15 | Example 16 | Example 17 | Example 18 | Example 19 | Example 20 | Example 21 | Example 22 | Example 23 |
|---|---|---|---|---|---|---|---|---|---|---|
| First agent | | | | | | | | | | |
| Behenyl alcohol | | | | | | | | | | |
| Arachyl alcohol | | | | | | | | | | |
| Stearyl alcohol | 3 | 3 | 3 | 3 | 3 | 3 | 3 | 3 | 3 | 3 |
| Cetyl alcohol | 4 | 4 | 4 | 4 | 4 | 4 | 4 | 4 | 4 | 4 |
| POE(30) cetyl ether | 2 | 2 | 2 | 2 | 2 | 2 | 2 | 2 | 2 | 2 |
| POE(2) cetyl ether | 1 | 1 | 1 | 1 | 1 | 1 | 1 | 1 | 1 | 1 |
| Stearyltrimethylammonium chloride | 1 | 1 | 1 | 1 | 1 | 1 | 1 | 1 | 1 | 1 |
| Sodium laureth sulfate | | | | | | | | | | |
| Cocamidopropyl betaine | | | | | | | | | | |
| Sodium chloride | 6 | 4 | | | | | | | 6 | |
| Hydroxyethyl cellulose | | | 0.5 | 2 | | | | | | 2 |
| Vaseline | 3 | 3 | 3 | 3 | 3 | 3 | 3 | 3 | 3 | 3 |
| Microcrystalline wax | | | | | | | | | | |
| Sodium sulfite | 0.1 | 0.1 | 0.1 | 0.1 | 0.1 | 0.1 | 0.1 | 0.1 | 0.1 | 0.1 |
| p-Phenylenediamine | 0.5 | 0.5 | 0.5 | 0.5 | 0.5 | 0.5 | 0.5 | 0.5 | 0.5 | 0.5 |
| m-Aminophenol | 0.4 | 0.4 | 0.4 | 0.4 | 0.4 | 0.4 | 0.4 | 0.4 | 0.4 | 0.4 |
| Resorcin | 0.5 | 0.5 | 0.5 | 0.5 | 0.5 | 0.5 | 0.5 | 0.5 | 0.5 | 0.5 |
| Ascorbic acid | 0.2 | 0.2 | 0.2 | 0.2 | 0.2 | 0.2 | 0.2 | 0.2 | 0.2 | 0.2 |
| 28% ammonia water | 4 | 4 | 4 | 4 | 4 | 4 | 4 | 4 | 4 | 4 |
| Purified water | Proper amount | Proper amount | Proper amount | Proper amount | Proper amount | Proper amount | Proper amount | Proper amount | Proper amount | Proper amount |
| Total | 100 | 100 | 100 | 100 | 100 | 100 | 100 | 100 | 100 | 100 |
| Second agent | | | | | | | | | | |
| Stearyl alcohol | 1 | 1 | 1 | 1 | 1 | 1 | 1 | 1 | 1 | 1 |
| Cetyl alcohol | 4 | 4 | 4 | 4 | 4 | 4 | 4 | 4 | 4 | 4 |
| POE(30) cetyl ether | 1 | 1 | 1 | 1 | 1 | 1 | 1 | 1 | 1 | 1 |
| POE(2) cetyl ether | 0.5 | 0.5 | 0.5 | 0.5 | 0.5 | 0.5 | 0.5 | 0.5 | 0.5 | 0.5 |
| Stearyltrimethylammonium chloride | 1 | 1 | 1 | 1 | 1 | 1 | 1 | 1 | 1 | 1 |
| Sodium chloride | | | | | 2 | 0.5 | | | 2 | |
| Hydroxyethyl cellulose | | | | | | | 1 | 2.5 | | 2.5 |
| Vaseline | 2 | 2 | 2 | 2 | 2 | 2 | 2 | 2 | 2 | 2 |
| Microcrystalline wax | | | | | | | | | | |
| Phenoxyethanol | 0.2 | 0.2 | 0.2 | 0.2 | 0.2 | 0.2 | 0.2 | 0.2 | 0.2 | 0.2 |
| Hydroxyethanediphosphonic acid | 0.1 | 0.1 | 0.1 | 0.1 | 0.1 | 0.1 | 0.1 | 0.1 | 0.1 | 0.1 |
| Tetrasodium hydroxyethanediphosphonate | 0.1 | 0.1 | 0.1 | 0.1 | 0.1 | 0.1 | 0.1 | 0.1 | 0.1 | 0.1 |
| 35% hydrogen peroxide | 16 | 16 | 16 | 16 | 16 | 16 | 16 | 16 | 16 | 16 |
| Purified water | Proper amount | Proper amount | Proper amount | Proper amount | Proper amount | Proper amount | Proper amount | Proper amount | Proper amount | Proper amount |
| Total | 100 | 100 | 100 | 100 | 100 | 100 | 100 | 100 | 100 | 100 |
| First agent: viscosity | 7200 | 10540 | 20600 | 29600 | 18580 | 18580 | 18580 | 18580 | 7200 | 29600 |
| Second agent: viscosity | 12650 | 12650 | 12650 | 12650 | 7400 | 10520 | 19850 | 28900 | 7400 | 28900 |
| First agent: surfactant amount | 4 | 4 | 4 | 4 | 4 | 4 | 4 | 4 | 4 | 4 |
| Second agent: surfactant amount | 2.5 | 2.5 | 2.5 | 2.5 | 2.5 | 2.5 | 2.5 | 2.5 | 2.5 | 2.5 |
| First agent: oil amount | 3 | 3 | 3 | 3 | 3 | 3 | 3 | 3 | 3 | 3 |
| Second agent: oil amount | 2 | 2 | 2 | 2 | 2 | 2 | 2 | 2 | 2 | 2 |
| First agent: (carbon number) × (mass) | 118 | 118 | 118 | 118 | 118 | 118 | 118 | 118 | 118 | 118 |
| Second agent: (carbon number) × (mass) | 82 | 82 | 82 | 82 | 82 | 82 | 82 | 82 | 82 | 82 |
| Degree of consumption of hydrogen peroxide | ⊚ | ⊚ | ⊚ | ⊚ | ⊚ | ⊚ | ⊚ | ⊚ | ⊚ | ⊚ |
| Uniform mixing properties after discharge | ⊚ | ⊚ | ⊚ | ⊚ | ⊚ | ⊚ | ⊚ | ⊚ | ⊚ | ⊚ |
| Brightness | ⊚ | ⊚ | ⊚ | ⊚ | ⊚ | ⊚ | ⊚ | ⊚ | ⊚ | ⊚ |

TABLE 12

| | Example | Example | Example | Example | Example | Example | Example | | | | | | | |
|---|---|---|---|---|---|---|---|---|---|---|---|---|---|---|
| | 24 | 25 | 26 | 27 | 28 | 29 | 30 | Example 31 | Example 32 | Example 33 | Example 34 | Example 35 | Example 36 | Example 37 |
| First agent | | | | | | | | | | | | | | |
| Stearyl alcohol | 3 | 3 | 3 | 3 | 3 | 3 | | 3 | 4 | 3 | 3 | 6 | 3 | 3 |
| Cetyl alcohol | 4 | 4 | 4 | 4 | 4 | 4 | 3 | 1 | 4 | 4 | 4 | 1 | 4 | 4 |
| POE(30) cetyl ether | 0.8 | 1.4 | 4 | 5 | 2 | 2 | 2 | 2 | 2 | 2 | 2 | 2 | 2 | 2 |
| POE(2) cetyl ether | 0.4 | 0.7 | 2 | 2.5 | 1 | 1 | 1 | 1 | 1 | 1 | 1 | 1 | 1 | 1 |
| Stearyltrimethylammonium | 0.4 | 0.7 | 2 | 2.5 | 1 | 1 | 1 | 1 | 1 | 1 | 1 | 1 | 1 | 0.5 |
| chloride | | | | | | | | | | | | | | |
| Sodium lauryl sulfate | | | | | | | | | | | | | | 0.5 |
| Vaseline | 3 | 3 | 3 | 3 | 1 | 6 | 3 | 3 | 3 | 3 | 7 | 3 | 3 | 3 |
| Stearyl stearate | | | | | | | 3 | | | | | | | |
| Sodium sulfite | 0.1 | 0.1 | 0.1 | 0.1 | 0.1 | 0.1 | 0.1 | 0.1 | 0.1 | 0.1 | 0.1 | 0.1 | 0.1 | 0.1 |
| p-Phenylenediamine | 0.5 | 0.5 | 0.5 | 0.5 | 0.5 | 0.5 | 0.5 | 0.5 | 0.5 | 0.5 | 0.5 | 0.5 | 0.5 | 0.5 |
| m-Aminophenol | 0.4 | 0.4 | 0.4 | 0.4 | 0.4 | 0.4 | 0.4 | 0.4 | 0.4 | 0.4 | 0.4 | 0.4 | 0.4 | 0.4 |
| Resorcin | 0.5 | 0.5 | 0.5 | 0.5 | 0.5 | 0.5 | 0.5 | 0.5 | 0.5 | 0.5 | 0.5 | 0.5 | 0.5 | 0.5 |
| Ascorbic acid | 0.2 | 0.2 | 0.2 | 0.2 | 0.2 | 0.2 | 0.2 | 0.2 | 0.2 | 0.2 | 0.2 | 0.2 | 0.2 | 0.2 |
| 28% ammonia water | 4 | 4 | 4 | 4 | 4 | 4 | 4 | 4 | 4 | 4 | 4 | 4 | 4 | 4 |
| Purified water | Proper | Proper | Proper | Proper | Proper | Proper | Proper | Proper | Proper | Proper | Proper | Proper | Proper | Proper |
| | amount | amount | amount | amount | amount | amount | amount | amount | amount | amount | amount | amount | amount | amount |
| Total | 100 | 100 | 100 | 100 | 100 | 100 | 100 | 100 | 100 | 100 | 100 | 100 | 100 | 100 |
| Second agent | | | | | | | | | | | | | | |
| Stearyl alcohol | 1 | 1 | 1 | 1 | 1 | 1 | 1 | 1 | 1 | 1 | 1 | 1 | 1 | 1 |
| Cetyl alcohol | 4 | 4 | 4 | 4 | 4 | 4 | 4 | 4 | 4 | 4 | 4 | 4 | 4 | 4 |
| POE(30) cetyl ether | 1 | 1 | 1 | 1 | 1 | 1 | 1 | 1 | 1 | 1 | 1 | 1 | 1 | 1 |
| POE(2) cetyl ether | 0.5 | 0.5 | 0.5 | 0.5 | 0.5 | 0.5 | 0.5 | 0.5 | 0.5 | 0.5 | 0.5 | 0.5 | 0.5 | 0.5 |
| Stearyltrimethylammonium | 1 | 1 | 1 | 1 | 1 | 1 | 1 | 1 | 1 | 1 | 1 | 1 | 0.5 | 1 |
| chloride | | | | | | | | | | | | | | |
| Sodium lauryl sulfate | | | | | | | | | | | | | 0.5 | |
| Vaseline | 2 | 2 | 2 | 2 | 0.5 | 3 | 2 | 2 | 2 | 2.8 | 1.5 | 2 | 2 | 2 |
| Phenoxyethanol | 0.2 | 0.2 | 0.2 | 0.2 | 0.2 | 0.2 | 0.2 | 0.2 | 0.2 | 0.2 | 0.2 | 0.2 | 0.2 | 0.2 |
| Hydroxyethanediphosphonic | 0.1 | 0.1 | 0.1 | 0.1 | 0.1 | 0.1 | 0.1 | 0.1 | 0.1 | 0.1 | 0.1 | 0.1 | 0.1 | 0.1 |
| acid | | | | | | | | | | | | | | |
| Tetrasodium | 0.1 | 0.1 | 0.1 | 0.1 | 0.1 | 0.1 | 0.1 | 0.1 | 0.1 | 0.1 | 0.1 | 0.1 | 0.1 | 0.1 |
| hydroxyethanediphosphonate | | | | | | | | | | | | | | |
| 35% hydrogen peroxide | 16 | 16 | 16 | 16 | 16 | 16 | 16 | 16 | 16 | 16 | 16 | 16 | 16 | 16 |
| Purified water | Proper | Proper | Proper | Proper | Proper | Proper | Proper | Proper | Proper | Proper | Proper | Proper | Proper | Proper |
| | amount | amount | amount | amount | amount | amount | amount | amount | amount | amount | amount | amount | amount | amount |
| Total | 100 | 100 | 100 | 100 | 100 | 100 | 100 | 100 | 100 | 100 | 100 | 100 | 100 | 100 |
| First agent: viscosity | 9800 | 8450 | 25600 | 26500 | 17800 | 17500 | 14200 | 13200 | 20850 | 18580 | 16350 | 21900 | 18580 | 16390 |
| Second agent: viscosity | 12650 | 12650 | 12650 | 12650 | 12900 | 12320 | 12650 | 12650 | 12650 | 12500 | 12650 | 12650 | 9800 | 12650 |
| First agent: surfactant | 1.6 | 2.8 | 8 | 10 | 4 | 4 | 4 | 4 | 4 | 4 | 4 | 4 | 4 | 4 |
| amount | | | | | | | | | | | | | | |
| Second agent: surfactant | 2.5 | 2.5 | 2.5 | 2.5 | 2.5 | 2.5 | 2.5 | 2.5 | 2.5 | 2.5 | 2.5 | 2.5 | 2.5 | 2.5 |
| amount | | | | | | | | | | | | | | |
| First agent: oil amount | 3 | 3 | 3 | 3 | 1 | 6 | 6 | 3 | 3 | 3 | 7 | 3 | 3 | 3 |
| Second agent: oil amount | 2 | 2 | 2 | 2 | 0.5 | 3 | 2 | 2 | 2 | 2.8 | 1.5 | 2 | 2.5 | 2 |
| First agent: | 118 | 118 | 118 | 118 | 118 | 118 | 48 | 70 | 136 | 118 | 118 | 124 | 118 | 118 |
| (carbon number) × (mass) | | | | | | | | | | | | | | |
| Second agent: | 82 | 82 | 82 | 82 | 82 | 82 | 82 | 82 | 82 | 82 | 82 | 82 | 82 | 82 |
| (carbon number) × (mass) | | | | | | | | | | | | | | |
| Degree of consumption | ⊚ | ⊚ | ⊚ | ⊚ | ⊚ | ⊚ | ⊚ | ⊚ | ⊚ | ⊚ | ⊚ | ⊚ | ⊚ | ⊚ |
| of hydrogen peroxide | | | | | | | | | | | | | | |
| Uniform mixing properties | ⊚ | ⊚ | ⊚ | ⊚ | ⊚ | ⊚ | ⊚ | ⊚ | ⊚ | ⊚ | ⊚ | ⊚ | ⊚ | ⊚ |
| after discharge | | | | | | | | | | | | | | |
| Brightness | ⊚ | ⊚ | ⊚ | ◯ | ◯ | ⊚ | ⊚ | ⊚ | ⊚ | ⊚ | ⊚ | ⊚ | ⊚ | ⊚ |

TABLE 13

| | Comparative | Comparative | Comparative | Comparative |
|---|---|---|---|---|
| | Example 1 | Example 2 | Example 3 | Example 4 |
| First agent | | | | |
| Behenyl alcohol | | | | |
| Arachyl alcohol | | | | |
| Stearyl alcohol | 3 | 3 | 3 | 3 |
| Cetyl alcohol | 4 | 4 | 4 | 4 |
| POE(30) cetyl ether | 2 | 2 | 2 | 2 |
| POE(2) cetyl ether | 1 | 1 | 1 | 1 |
| Stearyltrimethylammonium chloride | 1 | 1 | 1 | 1 |
| Sodium laureth sulfate | | | | |
| Cocamidopropyl betaine | | | | |
| Sodium chloride | 8 | | | |
| Hydroxyethyl cellulose | | 3 | | |
| Vaseline | 3 | 3 | 3 | 3 |
| Microcrystalline wax | | | | |
| Sodium sulfite | 0.1 | 0.1 | 0.1 | 0.1 |
| p-Phenylenediamine | 0.5 | 0.5 | 0.5 | 0.5 |
| m-Aminophenol | 0.4 | 0.4 | 0.4 | 0.4 |
| Resorcin | 0.5 | 0.5 | 0.5 | 0.5 |
| Ascorbic acid | 0.2 | 0.2 | 0.2 | 0.2 |
| 28% ammonia water | 4 | 4 | 4 | 4 |
| Purified water | Proper amount | Proper amount | Proper amount | Proper amount |
| Total | 100 | 100 | 100 | 100 |
| Second agent | | | | |
| Stearyl alcohol | 1 | 1 | 1 | 1 |
| Cetyl alcohol | 4 | 4 | 4 | 4 |
| POE(30) cetyl ether | 1 | 1 | 1 | 1 |
| POE(2) cetyl ether | 0.5 | 0.5 | 0.5 | 0.5 |
| Stearyltrimethylammonium chloride | 1 | 1 | 1 | 1 |
| Sodium chloride | | | 4 | |
| Hydroxyethyl cellulose | | | | 4 |
| Vaseline | 2 | 2 | 2 | 2 |
| Microcrystalline wax | | | | |
| Phenoxyethanol | 0.2 | 0.2 | 0.2 | 0.2 |
| Hydroxyethanediphosphonic acid | 0.1 | 0.1 | 0.1 | 0.1 |
| Tetrasodium hydroxyethanediphosphonate | 0.1 | 0.1 | 0.1 | 0.1 |
| 35% hydrogen peroxide | 16 | 16 | 16 | 16 |
| Purified water | Proper amount | Proper amount | Proper amount | Proper amount |
| Total | 100 | 100 | 100 | 100 |
| First agent: viscosity | 5200 | 35000 | 18580 | 18580 |
| Second agent: viscosity | 12650 | 12650 | 4800 | 35800 |
| First agent: surfactant amount | 4 | 4 | 4 | 4 |
| Second agent: surfactant amount | 2.5 | 2.5 | 2.5 | 2.5 |
| First agent: oil amount | 3 | 3 | 3 | 3 |
| Second agent: oil amount | 2 | 2 | 2 | 2 |
| First agent: (carbon number) × (mass) | 118 | 118 | 118 | 118 |
| Second agent: (carbon number) × (mass) | 82 | 82 | 82 | 82 |
| Degree of consumption of hydrogen peroxide | X | X | X | X |
| Uniform mixing properties after discharge | ◯ | ◯ | ◯ | ◯ |
| Brightness | ⊚ | ⊚ | ⊚ | ⊚ |

### Fourth Embodiment

### [Hair Cosmetic Material]

First of all, the hair cosmetic material in the fourth embodiment of the present invention is explained centering on a first agent and a second agent. Details of main components of a first agent and a second agent mentioned in this embodiment are described later.

The hair cosmetic material in the fourth embodiment is constituted to include at least a first agent and a second agent. These first agent and second agent are respectively filled in a space for filling the first agent and a space for filling the second agent, each of which is, for example, a bag-like body, in a separate filling/same pressure discharge-type double structure container as described later. Each of the first agent and the second agent of the hair cosmetic material is an emulsified creamy dosage form and is discharged in the same cream state from the double structure container.

As the hair cosmetic material, such a two-agent type composed of the first agent and the second agent is exemplified; however, a multi-agent type such as a three-agent type, in which a third agent or the like according to an appropriate preparation is further added, is also included. The dosage form of the third agent or the like to be added is not particularly limited. In the case where the hair cosmetic material is a three-agent type or the like, in general, the third agent or the like is attached to the double structure container having the first agent and the second agent filled therein, whereby it becomes a constituent element of a hair cosmetic material product as a commodity.

A category of the hair cosmetic material is not limited so long as it is constituted to include at least the first agent and the second agent. However, preferably, examples thereof include an oxidation hair dyeing agent, a hair bleaching agent, and a hair dedyeing agent. Although these are common from the standpoint of including the first agent containing an alkali agent and the second agent containing an oxidizing agent, the oxidation hair dyeing agent further includes an oxidation dye. The oxidation dye is composed of a principal intermediate, or composed of a principal intermediate and a coupler; however, as the case may be, a direct dye is further added. In the hair dedyeing agent, a persulfate is added as an oxidation aid in addition to the alkali agent.

In the hair cosmetic material of the fourth embodiment, each of the first agent and the second agent has a viscosity ratio V₃₀/V₁₂ of 0.5 or more, and more preferably 0.55 or more, the ratio being a ratio of a viscosity V₃₀ at 30 rpm to a viscosity V₁₂ at 12 rpm as measured by using a B-type rotational viscometer at 25° C. Furthermore, in the hair cosmetic material of the present invention, the viscosity ratio V₃₀/V₁₂ (f) in the first agent and the viscosity ratio V₃₀/V₁₂(s) in the second agent is preferably in the range not exceeding 1.3 times each other, and more preferably in the range not exceeding 1.2 times each other.

The viscosity V₃₀ at 30 rpm and the viscosity V₁₂ at 12 rpm refer to values obtained as a measured viscosity V₃₀ (mPa·s) at 30 rpm and a measured viscosity V₁₂ (mPa·s) at 12 rpm, respectively as measured at 25° C. with a so-called B-type rotational viscometer by using a No. 4 rotor.

The first agent of the hair cosmetic material can contain at least one nonionic surfactant in a total content falling within the range of preferably from 1 to 10% by mass, and more preferably from 2 to 8% by mass. At least one nonionic surfactant may also be properly compounded in the second agent of the hair cosmetic material.

The first agent of the hair cosmetic material can further contain at least one oily component in a total content of preferably 1% by mass or more, and more preferably 2% by mass or more. At least one oily component may also be properly compounded in the second agent of the hair cosmetic material.

Preferably, the first agent of the hair cosmetic material can further contain at least one higher alcohol. The “higher alcohol” as referred to in the fourth embodiment of the present application refers to a saturated or unsaturated, linear or branched monohydric alcohol having 12 or more carbon atoms. At least one higher alcohol may also be properly compounded in the second agent of the hair cosmetic material.

In the first agent of the hair cosmetic material, though a ratio C(n)/C(a) of a total content C(n) of the at least one nonionic surfactant to a total content C(a) of the at least one higher alcohol in terms of a mass % unit is not limited, it is preferably within the range of from 0.3 to 1.0, and more preferably within the range of from 0.35 to 0.9.

In addition, preferably, the first agent of the hair cosmetic material can further contain at least one ionic surfactant. Examples of the ionic surfactant include a cationic surfactant, an anionic surfactant, and an ampholytic surfactant.

It is preferred that at least one ionic surfactant is contained in a total content of 0.2 to 1% by mass in the first agent of the hair cosmetic material from the standpoints of an enhancement of emulsion stability and keeping of rheology properties with time in the first agent. Furthermore, though a ratio C(i)/C(n) of a total content C(i) of the at least one ionic surfactant to a total content C(n) of the at least one nonionic surfactant in terms of a mass % unit is not limited, it is preferably 1.5 or less, and more preferably 1.0 or less.

### [Principal Components of Hair Cosmetic Material]

Next, embodiments of the essential components and the principal arbitrary compounding components to be contained in the hair cosmetic material in the fourth embodiment are successively described in detail.

### (Alkali Agent)

The alkali agent in the fourth embodiment is identical with that exemplified in the third embodiment, and hence, its explanation is omitted. It is to be noted that though a content of the alkali agent in the first agent is not limited, it is, for example, 0.1 to 15% by mass, and more preferably 1 to 12% by mass.

### (Oxidizing Agent and Oxidation Aid, Oxidation Dye and Direct Dye)

The oxidizing agent, the oxidation aid, the oxidation dye, and the direct dye in the fourth embodiment are also identical with those exemplified in the third embodiment, and hence, their explanation is omitted.

### (Surfactant)

In the first agent of the hair cosmetic material, at least one nonionic surfactant can be compounded as a preferred arbitrary component in a total content within the foregoing range. In addition, in the first agent of the hair cosmetic material, at least one ionic surfactant of any one of a cationic surfactant, an anionic surfactant, and an ampholytic surfactant can also be compounded as a preferred arbitrary component.

Although a total compounding amount of the at least one ionic surfactant in the first agent is not particularly limited, for example, it can be allowed to fall within the range of from 0.1 to 3% by mass in the first agent. However, in particular, in the case where at least one nonionic surfactant is compounded in the first agent, it is preferred that the ionic surfactant in the first agent is compounded together with the nonionic surfactant. In the case of such a combined use, it is preferred to set the total content of the ionic surfactant in such a manner that the ratio C(i)/C(n) of the total content C(i) of the ionic surfactant to the total content C(n) of the nonionic surfactant in terms of a mass % unit becomes the foregoing value.

In the second agent of the hair cosmetic material, at least one nonionic surfactant and/or at least one ionic surfactant may also be compounded in an amount falling within an appropriate quantitative range.

Examples of the nonionic surfactant include ether types and ester types.

As the ether-type and ester-type nonionic surfactants, the same surfactants as those exemplified in the third embodiment can be used, and hence, their explanation is omitted.

In addition, as the cationic surfactant, the anionic surfactant, and the ampholytic surfactant, the same surfactants as those exemplified in the third embodiment can be used, and hence, their explanation is omitted.

### (Oily Component)

In the first agent of the hair cosmetic material, at least one oily component can be compounded in a total content within the foregoing range as a preferred arbitrary component. In the second agent of the hair cosmetic material, an oily component may also be compounded in an amount falling within an appropriate quantitative range.

Examples of the oily component include a fat and oil, a wax, a higher fatty acid, an alkyl glyceryl ether, an ester, a silicone, a hydrocarbon, and the like.

Examples of the fat and oil include olive oil, rose hip oil, camellia oil, shea butter, macadamia nut oil, almond oil, tea seed oil, safflower oil, sunflower oil, soybean oil, cottonseed oil, sesame oil, beef tallow, cacao butter, corn oil, peanut oil, rapeseed oil, rice bran oil, rice germ oil, wheat germ oil, Coix lacryma-jobi seed oil, grape seed oil, avocado oil, carrot oil, castor oil, linseed oil, coconut oil, mink oil, egg yolk oil, and the like.

As the wax, the higher fatty acid, the ester, the silicone, the hydrocarbon, each of which is the oily component, the same materials as those exemplified as the oily component in the third embodiment can be used, and hence, their explanation is omitted.

### (Higher Alcohol)

In the first agent of the hair cosmetic material, at least one higher alcohol can be compounded as a preferred arbitrary component. Although a total compounding amount of the higher alcohol in the first agent is not particularly limited, for example, it can be allowed to fall within the range of from 0.5 to 10% by mass in the first agent. However, in particular, in the case where at least one nonionic surfactant is compounded in the first agent, it is preferred that the at least one higher alcohol in the first agent is compounded together with the nonionic surfactant. In the case of such a combined use, it is preferred to set the total content of the higher alcohol in such a manner that the ratio C(n)/C(a) of the total content C(n) of the nonionic surfactant to the total content C(a) of the higher alcohol in the first agent in terms of a mass % unit becomes the foregoing value.

In the second agent of the hair cosmetic material, at least one higher alcohol may also be compounded in an amount falling within an appropriate quantitative range.

Specifically, examples of the linear, saturated higher alcohol may include lauryl alcohol, myristyl alcohol, cetyl alcohol (cetanol), stearyl alcohol, arachyl alcohol, and behenyl alcohol. Besides, examples of the branched, saturated higher alcohol may include isostearyl alcohol, 2-hexyldodecanol, 2-octyldodecanol, and the like, and examples of the unsaturated higher alcohol may include oleyl alcohol and the like.

Among the foregoing, lauryl alcohol, myristyl alcohol, cetyl alcohol (cetanol), stearyl alcohol, arachyl alcohol, and behenyl alcohol, all of which are a linear, saturated higher alcohol having a carbon number in the range of from 12 to 22, are especially preferred.

### [Other Arbitrary Compounding Components of Hair Cosmetic Material]

In the first agent and/or the second agent of the hair cosmetic material, in addition to the above-described various components, for example, a cationic polymer, a solubilizing agent, a water-soluble polymer compound, a saccharide, an antiseptic, a stabilizer, a pH adjuster, a plant extract, a crude drug extract, a vitamin, a perfume, an antioxidant, an ultraviolet light absorber, a chelating agent, or the like can be arbitrarily compounded. Some of them are hereunder specifically described.

### (Cationic Polymer)

As the cationic polymer in the fourth embodiment, the same materials as those exemplified as the cationic polymer of the “Other arbitrary compounding components of hair cosmetic material” section in the third embodiment can be used, and hence, its explanation is omitted.

### (Solubilizing Agent)

The solubilizing agent is compounded for the purpose of rendering each of the agents of the hair cosmetic material composition liquid. Examples of the solubilizing agent include water and organic solvents. Examples of the organic solvent include monohydric alcohols having 1 to 3 carbon atoms (lower alcohols), glycols and glycerins as polyhydric alcohols, and diethylene glycol lower alkyl ethers. Examples of the monohydric alcohol having 1 to 3 carbon atoms include methanol, ethanol, propanol, and isopropanol. Examples of the glycol include ethylene glycol, diethylene glycol, triethylene glycol, propylene glycol, dipropylene glycol, isoprene glycol, hexylene glycol, and 1,3-butylene glycol. Examples of the glycerin include glycerin, diglycerin, and polyglycerin. Examples of the diethylene glycol lower alkyl ether include diethylene glycol monoethyl ether (ethyl carbitol).

### (Water-Soluble Polymer Compound)

As the water-soluble polymer compound, anionic, nonionic, or ampholytic polymer compounds, exclusive of the above-described cationic polymers can be used. Examples thereof include a carboxyvinyl polymer, a diallyl quaternary ammonium salt/acrylic acid copolymer, and the like.

### [Double Structure Container and Hair Cosmetic Material Product]

### (Double Structure Container)

A double structure container which is used in the fourth embodiment is identical with that explained in the third embodiment (see FIG. 2), and hence, its explanation is omitted.

### (Hair Cosmetic Material Product)

The hair cosmetic material product of the present invention is one in which the first agent and the second agent of the hair cosmetic material are respectively filled in the inner container 2 and the inner container 3 in the double structure container 1 as shown in FIG. 2. As the case may be, a third agent is attached as an additional constituent element of the hair cosmetic material product.

In the double structure container 1 having the first agent and the second agent filled therein, both of the first agent and the second agent in the inner containers 2 and 3 always receive a discharge pressure by the compressed gas in the propellant filling space 9. Then, when the actuator 6 is subjected to press-down resisting to a pushing force of the pushing spring, the valve stem becomes in an “open” state, whereby the first agent and the second agent are simultaneously discharged. When a pressing force against the actuator 6 is released, the valve stem becomes in a “closed” state, whereby the discharges of the first agent and the second agent are simultaneously stopped.

### EXAMPLES

Next, Examples and Comparative Examples of the fourth embodiment are hereunder explained. It should be construed that the technical scope of the present invention is not limited by the following Examples and Comparative Examples.

### [Preparation of Hair Cosmetic Material]

A first agent and a second agent of each of two-agent type oxidation hair dyeing agents according to Examples 1 to 21 and Comparative Examples 1 to 6 of the fourth embodiment, each having a composition shown in the following Tables 14 to 17, respectively, were prepared according to the conventional procedure. All of these first and second agents are an emulsified preparation in a cream state. In the tables, the numerical value showing the content of each component is a numerical value in terms of a mass % unit in the first agent or the second agent.

Next, in each of the tables, with respect to each of the first agent and the second agent, the term “Viscosity at 30 rpm (mPa·s) after 3 days” is a viscosity value (mPa·s) as measured in such a manner that the first agent or the second agent according to each of the Examples or each of the Comparative Examples was stabilized by allowing to stand for 3 days after the preparation, and thereafter, the viscosity was measured by using a BL-type viscometer, VISCOMETER that is a B-type viscometer available from Toki Sangyo Co., Ltd. at 25° C. under measurement conditions of using a No. 4 rotor for one minute at a rotating rate of 30 rpm/min. In addition, the term “Viscosity at 12 rpm (mPa·s) after 3 days” is a viscosity value (mPa·s) as measured in such a manner that the first agent or the second agent according to each of the Examples or each of the Comparative Examples was stabilized by allowing to stand for 3 days after the preparation, and thereafter, the viscosity was measured by using a BL-type viscometer, VISCOMETER that is a B-type viscometer available from Toki Sangyo Co., Ltd. at 25° C. under measurement conditions of using a No. 4 rotor for one minute at a rotating rate of 12 rpm/min.

In addition, in each of the tables, with respect to each of the first agent and the second agent, the terms “V₃₀/V₁₂(f)” and “V₃₀/V₁₂(s)” each express a value of the “V₃₀/V₁₂” ratio of the measured viscosity V₃₀ to the measured viscosity V₁₂ with respect to each of the first agent and the second agent according to each of the Examples and Comparative Examples. With respect to whether or not the values of both of the viscosity ratio V₃₀/V₁₂(f) of the first agent and the viscosity ratio V₃₀/V₁₂(s) of the second agent do not exceed 1.3 times each other, the calculation results are described in the “(V₃₀/V₁₂)) (V₃₀/V₁₂(s)” row and the “(V₃₀/V₁₂(s))/(V₃₀/V₁₂(f))” row, respectively. It is to be noted that in the “V₃₀/V₁₂(f)” and “V₃₀/V₁₂(s)” rows, numerical values obtained by performing round off to two decimal places are entered; however, in calculating the “(V₃₀/V₁₂(f))/(V₃₀/V₁₂(s))” and “(V₃₀/V₁₂(s))/(V₃₀/V₁₂(f))”, the calculation is made by using numerical values of “V₃₃/V₁₂(f)” and “V₃₀/V₁₂(s)” which is not rounded off to two decimal places.

Next, in each of the tables, the terms “Amount of nonionic surfactant” and “Amount of oily component” regarding the first agent each express a numerical value of a total content of each of the nonionic surfactant and the oily component in the first agent according to each of the Examples and Comparative Examples in terms of a mass % unit.

Next, in each of the tables, the term “C(n)/C(a)” regarding the first agent expresses a value of the ratio “C(n)/C(a)” of the total content C(n) of the nonionic surfactant to the total content C(a) of the higher alcohol in the first agent in terms of a mass % unit.

Furthermore, in the each of the tables, the term “C(i)/C(n)” regarding the first agent expresses a value of the ratio “C(i)/C(n)” of the total content C(i) of the ionic surfactant in the first agent to the total content C(n) of the nonionic surfactant in the first agent in terms of a mass % unit.

### [Evaluation of Hair Cosmetic Material]

### (Emulsion Stability)

Immediately after preparing the first agent in a cream state according to each of the Examples or each of the Comparative Examples, the resulting first agent was transferred into a thermostatic chamber of 60 degree C. and allowed to stand. A time required until phase separation between an oil phase and an aqueous phase was generated was measured, thereby evaluating the emulsion stability. The case where a time of 72 hours or more was required until the phase separation was generated was evaluated as “⊙”; the case where a time of 24 hours or more and less than 72 hours was required until the phase separation was generated was evaluated as “◯”; the case where a time of 12 hours or more and less than 24 hours was required until the phase separation was generated was evaluated as “Δ”; and the case where the phase separation was generated within less than 12 hours was evaluated as “×”. The evaluation results are described in the “Emulsion stability” row in each of the tables.

### (Hair Dyeing Performance)

After preparing the first agent and the second agent in a cream state according to each of the Examples or each of the Comparative Examples, a hair dyeing treatment was performed by uniformly mixing the both agents by using a brush and uniformly applying 2 mL of the mixture to a grizzled hair bundle sample for evaluation having a length of 10 cm, followed by allowing the resultant to stand for 30 minutes. Thereafter, the hair bundle sample was washed with water, dried, and then evaluated for the effect of hair dyeing by 10 panelists. As for evaluation criteria, the case where the hair dyeing effect was very good was evaluated as “⊙”; the case where the hair dyeing effect was good was evaluated as “◯”; the case where the hair dyeing effect was not bad but could not be said to be good was evaluated as “Δ”; and the case where the hair dyeing effect was bad was evaluated as “×”.

In all of the cases, the evaluation made by the largest number of the ten panelists was adopted. In the case where there were two or more the evaluations made by the largest number of the ten panelists, the lower evaluation was adopted. The evaluation results are described in the “Hair dyeing performance” row in each of the tables.

### (Equal Amount Discharge Properties)

After filling the same already-known amounts of the first agent and the second agent in a cream state according to each of the Examples or each of the Comparative Examples in a separate filling/same pressure discharge-type double structure container, a simultaneous discharge operation of the first agent and the second agent was performed until just half an amount (on a mass % basis) of the filling amount of the second agent was discharged, and at that point of time, the simultaneous discharge operation was stopped.

Then, the discharge amount of the first agent on a mass basis up to that point of time was measured. The case where a ratio on a mass basis of the discharge amount of the first agent to the discharge amount of the second agent was 0.9 or more and 1.1 or less was evaluated as “⊙”; the case where the ratio was 0.8 or more and less than 0.9, or more than 1.1 and 1.2 or less, was evaluated as “◯”; the case where the ratio was 0.7 or more and less than 0.8, or more than 1.2 and 1.3 or less, was evaluated as “Δ”; and the case where the ratio was less than 0.7, or more than 1.3, was evaluated as “×”. The evaluation results are described in the “Equal amount discharge properties” row in each of the tables.

TABLE 14

| | Example 1 | Example 2 | Example 3 | Example 4 | Example 5 | Example 6 | Example 7 | Example 8 | Example 9 |
|---|---|---|---|---|---|---|---|---|---|
| First agent | | | | | | | | | |
| Behenyl alcohol | | | | | | | | | |
| Arachyl alcohol | | 2 | | | | | | | |
| Stearyl alcohol | 3 | 5 | 1 | 5 | 3 | 3 | 3 | 3 | 3 |
| Cetanol | 4 | | 2 | 6 | 4 | 4 | 4 | 4 | 4 |
| POE(30) cetyl ether | 2 | 2 | 2 | 2 | 2 | 2 | 2 | 2 | 2 |
| POE(2) cetyl ether | 1 | 1 | 1 | 1 | 1 | 1 | 1 | 1 | 1 |
| POE(2) stearyl ether | | | | | | | | | |
| POE(50) oleyl ether | | | | | | | | | |
| Stearyltrimethylammonium chloride | 1 | 1 | 1 | 1 | | | 1 | 1 | 1 |
| Sodium lauryl sulfate | | | | | 1 | | | | |
| Coconut oil fatty acid amidopropyl betaine | | | | | | 1 | | | |
| Vaseline | 3 | 3 | 3 | 3 | 3 | 3 | | | |
| Microcrystalline wax | | | | | | | 3 | | |
| Liquid paraffin | | | | | | | | 3 | |
| Cetyl octanoate | | | | | | | | | 3 |
| Sodium sulfite | 0.1 | 0.1 | 0.1 | 0.1 | 0.1 | 0.1 | 0.1 | 0.1 | 0.1 |
| p-Phenylenediamine | 0.5 | 0.5 | 0.5 | 0.5 | 0.5 | 0.5 | 0.5 | 0.5 | 0.5 |
| m-Aminophenol | 0.4 | 0.4 | 0.4 | 0.4 | 0.4 | 0.4 | 0.4 | 0.4 | 0.4 |
| Resorcin | 0.5 | 0.5 | 0.5 | 0.5 | 0.5 | 0.5 | 0.5 | 0.5 | 0.5 |
| Ascorbic acid | 0.2 | 0.2 | 0.2 | 0.2 | 0.2 | 0.2 | 0.2 | 0.2 | 0.2 |
| 28% ammonia water | 4 | 4 | 4 | 4 | 4 | 4 | 4 | 4 | 4 |
| Purified water | Proper | Proper | Proper | Proper | Proper | Proper | Proper | Proper | Proper |
| | amount | amount | amount | amount | amount | amount | amount | amount | amount |
| Total | 100 | 100 | 100 | 100 | 100 | 100 | 100 | 100 | 100 |
| Viscosity at 30 rpm (mPa · s) after 3 days | 11130 | 10920 | 6230 | 14490 | 6340 | 4050 | 6060 | 5980 | 6290 |
| Viscosity at 12 rpm (mPa · s) after 3 days | 18580 | 18340 | 10870 | 25480 | 10850 | 7920 | 10500 | 10800 | 11580 |
| V₃₀/V₁₂(f) | 0.60 | 0.60 | 0.57 | 0.57 | 0.58 | 0.51 | 0.58 | 0.55 | 0.54 |
| Amount of nonionic surfactant | 3 | 3 | 3 | 3 | 3 | 3 | 3 | 3 | 3 |
| Amount of oily component | 3 | 3 | 3 | 3 | 3 | 3 | 3 | 3 | 3 |
| C(n)/C(a) | 0.43 | 0.43 | 1.00 | 0.27 | 0.43 | 0.43 | 0.43 | 0.43 | 0.43 |
| C(i)/C(n) | 0.33 | 0.33 | 0.33 | 0.33 | 0.33 | 0.33 | 0.33 | 0.33 | 0.33 |
| Emulsion stability | ⊚ | ⊚ | ⊚ | ◯ | ⊚ | ⊚ | ⊚ | ⊚ | ⊚ |
| Hair dyeing performance | ⊚ | ⊚ | ⊚ | ⊚ | ⊚ | ⊚ | ⊚ | ⊚ | ⊚ |
| Second agent | | | | | | | | | |
| Stearyl alcohol | 1 | 1 | 1 | 1 | 1 | 1 | 1 | 1 | 1 |
| Cetanol | 4 | 4 | 4 | 4 | 4 | 4 | 4 | 4 | 4 |
| POE(30) cetyl ether | 1 | 1 | 1 | 1 | 1 | 1 | 1 | 1 | 1 |
| POE(2) cetyl ether | 0.5 | 0.5 | 0.5 | 0.5 | 0.5 | 0.5 | 0.5 | 0.5 | 0.5 |
| Stearyltrimethylammonium chloride | 4 | 4 | 4 | 4 | 4 | 4 | 4 | 4 | 4 |
| Vaseline | 2 | 2 | 2 | 2 | 2 | 2 | 2 | 2 | 2 |
| Microcrystalline wax | | | | | | | | | |
| Phenoxyethanol | 0.2 | 0.2 | 0.2 | 0.2 | 0.2 | 0.2 | 0.2 | 0.2 | 0.2 |
| Hydroxyethanediphosphonic acid | 0.15 | 0.15 | 0.15 | 0.15 | 0.15 | 0.15 | 0.15 | 0.15 | 0.15 |
| Tetrasodium hydroxyethanediphosphonate | 0.3 | 0.3 | 0.3 | 0.3 | 0.3 | 0.3 | 0.3 | 0.3 | 0.3 |
| 35% hydrogen peroxide | 15.7 | 15.7 | 15.7 | 15.7 | 15.7 | 15.7 | 15.7 | 15.7 | 15.7 |
| Purified water | Proper | Proper | Proper | Proper | Proper | Proper | Proper | Proper | Proper |
| | amount | amount | amount | amount | amount | amount | amount | amount | amount |
| Total | 100 | 100 | 100 | 100 | 100 | 100 | 100 | 100 | 100 |
| Viscosity at 30 rpm (mPa · s) after 3 days | 7640 | 7640 | 7640 | 7640 | 7640 | 7640 | 7640 | 7640 | 7640 |
| Viscosity at 12 rpm (mPa · s) after 3 days | 12650 | 12650 | 12650 | 12650 | 12650 | 12650 | 12650 | 12650 | 12650 |
| V₃₀/V₁₂(s) | 0.60 | 0.60 | 0.60 | 0.60 | 0.60 | 0.60 | 0.60 | 0.60 | 0.60 |
| (V₃₀/V₁₂(f))/(V₃₀/V₁₂(s)) | 0.99 | 0.99 | 0.95 | 0.94 | 0.97 | 0.85 | 0.96 | 0.92 | 0.90 |
| (V₃₀/V₁₂(s))/(V₃₀/V₁₂(f)) | 1.01 | 1.01 | 1.05 | 1.06 | 1.03 | 1.18 | 1.05 | 1.09 | 1.11 |
| Equal amount discharge properties | ⊚ | ⊚ | ⊚ | ⊚ | ⊚ | ◯ | ⊚ | ⊚ | ⊚ |

TABLE 15

| | Example 10 | Example 11 | Example 12 | Example 13 | Example 14 |
|---|---|---|---|---|---|
| First agent | | | | | |
| Behenyl alcohol | | | | | 1 |
| Arachyl alcohol | | | | | |
| Stearyl alcohol | 1 | 3 | 3 | 3 | |
| Cetanol | 2 | 4 | 4 | 4 | 6 |
| POE(30) cetyl ether | 1 | 5 | 1 | 2 | 2 |
| POE(2) cetyl ether | 0.5 | 2 | 0.5 | 1 | 1 |
| POE(2) stearyl ether | | | | | |
| POE(50) oleyl ether | | | | | |
| Stearyltrimethylammonium chloride | 1 | 1 | 1 | 1 | 1 |
| Sodium lauryl sulfate | | | | | |
| Coconut oil fatty acid amidopropyl betaine | | | | | |
| Vaseline | 3 | 3 | 3 | | 3 |
| Microcrystalline wax | | | | | |
| Liquid paraffin | | | | | |
| Cetyl octanoate | | | | | |
| Sodium sulfite | 0.1 | 0.1 | 0.1 | 0.1 | 0.1 |
| p-Phenylenediamine | 0.5 | 0.5 | 0.5 | 0.5 | 0.5 |
| m-Aminophenol | 0.4 | 0.4 | 0.4 | 0.4 | 0.4 |
| Resorcin | 0.5 | 0.5 | 0.5 | 0.5 | 0.5 |
| Ascorbic acid | 0.2 | 0.2 | 0.2 | 0.2 | 0.2 |
| 28% ammonia water | 4 | 4 | 4 | 4 | 4 |
| Purified water | Proper amount | Proper amount | Proper amount | Proper amount | Proper amount |
| Total | 100 | 100 | 100 | 100 | 100 |
| Viscosity at 30 rpm (mPa · s) after 3 days | 1930 | 9030 | 4570 | 11130 | 11500 |
| Viscosity at 12 rpm (mPa · s) after 3 days | 3820 | 18100 | 8480 | 18580 | 18380 |
| V₃₀/V₁₂(f) | 0.51 | 0.50 | 0.54 | 0.60 | 0.63 |
| Amount of nonionic surfactant | 1.5 | 7 | 1.5 | 3 | 3 |
| Amount of oily component | 3 | 3 | 3 | 0 | 3 |
| C(n)/C(a) | 0.50 | 1.00 | 0.21 | 0.43 | 0.43 |
| C(i)/C(n) | 0.67 | 0.14 | 0.67 | 0.33 | 0.33 |
| Emulsion stability | ◯ | ⊚ | ⊚ | ⊚ | ⊚ |
| Hair dyeing performance | ⊚ | ⊚ | ⊚ | ◯ | ⊚ |
| Second agent | | | | | |
| Stearyl alcohol | 1 | 1 | 1 | 1 | 1 |
| Cetanol | 4 | 4 | 4 | 4 | 4 |
| POE(30) cetyl ether | 1 | 1 | 1 | 1 | 1 |
| POE(2) cetyl ether | 0.5 | 0.5 | 0.5 | 0.5 | 0.5 |
| Stearyltrimethylammonium chloride | 4 | 4 | 4 | 4 | 4 |
| Vaseline | 2 | 2 | 2 | 2 | 2 |
| Microcrystalline wax | | | | | |
| Phenoxyethanol | 0.2 | 0.2 | 0.2 | 0.2 | 0.2 |
| Hydroxyethanediphosphonic acid | 0.15 | 0.15 | 0.15 | 0.15 | 0.15 |
| Tetrasodium hydroxyethanediphosphonate | 0.3 | 0.3 | 0.3 | 0.3 | 0.3 |
| 35% hydrogen peroxide | 15.7 | 15.7 | 15.7 | 15.7 | 15.7 |
| Purified water | Proper amount | Proper amount | Proper amount | Proper amount | Proper amount |
| Total | 100 | 100 | 100 | 100 | 100 |
| Viscosity at 30 rpm (mPa · s) after 3 days | 7640 | 7640 | 7640 | 7640 | 7640 |
| Viscosity at 12 rpm (mPa · s) after 3 days | 12650 | 12650 | 12650 | 12650 | 12650 |
| V₃₀/V₁₂(s) | 0.60 | 0.60 | 0.60 | 0.60 | 0.60 |
| (V₃₀/V₁₂(f))/(V₃₀/V₁₂(s)) | 0.84 | 0.83 | 0.89 | 0.99 | 1.04 |
| (V₃₀/V₁₂(s))/(V₃₀/V₁₂(f)) | 1.20 | 1.21 | 1.12 | 1.01 | 0.97 |
| Equal amount discharge properties | ◯ | ◯ | ⊚ | ⊚ | ⊚ |

TABLE 16

| | Example 15 | Example 16 | Example 17 | Example 18 | Example 19 | Example 20 | Example 21 |
|---|---|---|---|---|---|---|---|
| First agent | | | | | | | |
| Behenyl alcohol | | | | | | | |
| Arachyl alcohol | | | | | | | |
| Stearyl alcohol | 3 | 3 | 3 | 3 | 2 | 3 | 5 |
| Cetanol | 4 | 4 | 4 | 4 | 3 | 4 | 5 |
| POE(30) cetyl ether | 2 | | | 2 | 1 | 0.8 | 3 |
| POE(2) cetyl ether | | 1 | 1 | 1 | 0.5 | 0.6 | 3 |
| POE(2) stearyl ether | 1 | | | | | | 2 |
| POE(50) oleyl ether | | 2 | 2 | | | | 2 |
| Stearyltrimethylammonium | 1 | 1 | 1 | 1 | 1 | 1 | 1 |
| chloride | | | | | | | |
| Sodium lauryl sulfate | | | | | | 1 | |
| Coconut oil fatty acid | | | | | | | |
| amidopropyl betaine | | | | | | | |
| Vaseline | 3 | 3 | 3 | | 3 | 3 | 3 |
| Microcrystalline wax | | | | 3 | | | |
| Liquid paraffin | | | | | | | |
| Cetyl octanoate | | | | 6 | | | |
| Sodium sulfite | 0.1 | 0.1 | 0.1 | 0.1 | 0.1 | 0.1 | 0.1 |
| p-Phenylenediamine | 0.5 | 0.5 | 0.5 | 0.5 | 0.5 | 0.5 | 0.5 |
| m-Aminophenol | 0.4 | 0.4 | 0.4 | 0.4 | 0.4 | 0.4 | 0.4 |
| Resorcin | 0.5 | 0.5 | 0.5 | 0.5 | 0.5 | 0.5 | 0.5 |
| Ascorbic acid | 0.2 | 0.2 | 0.2 | 0.2 | 0.2 | 0.2 | 0.2 |
| 28% ammonia water | 4 | 4 | 4 | 4 | 4 | 4 | 4 |
| Purified water | Proper amount | Proper amount | Proper amount | Proper amount | Proper amount | Proper amount | Proper amount |
| Total | 100 | 100 | 100 | 100 | 100 | 100 | 100 |
| Viscosity at 30 rpm (mPa · s) | 11030 | 11850 | 11850 | 14350 | 4850 | 7980 | 26850 |
| after 3 days | | | | | | | |
| Viscosity at 12 rpm (mPa · s) | 17890 | 18030 | 18030 | 22850 | 7850 | 12200 | 39850 |
| after 3 days | | | | | | | |
| V₃₀/V₁₂(f) | 0.62 | 0.66 | 0.66 | 0.63 | 0.62 | 0.65 | 0.67 |
| Amount of nonionic surfactant | 3 | 3 | 3 | 3 | 1.5 | 1.4 | 10 |
| Amount of oily component | 3 | 3 | 3 | 9 | 3 | 3 | 3 |
| C(n)/C(a) | 0.43 | 0.43 | 0.43 | 0.43 | 0.30 | 0.20 | 1.00 |
| C(i)/C(n) | 0.33 | 0.33 | 0.33 | 0.33 | 0.67 | 1.43 | 0.10 |
| Emulsion stability | ⊚ | ⊚ | ⊚ | ◯ | ⊚ | ◯ | ⊚ |
| Hair dyeing performance | ⊚ | ⊚ | ⊚ | ⊚ | ⊚ | ⊚ | ⊚ |
| Second agent | | | | | | | |
| Stearyl alcohol | 1 | 1 | 1 | 1 | 1 | 1 | 1 |
| Cetanol | 4 | 4 | 4 | 4 | 4 | 4 | 4 |
| POE(30) cetyl ether | 1 | 1 | 1.5 | 1 | 1 | 1 | 1 |
| POE(2) cetyl ether | 0.5 | 0.5 | 1 | 0.5 | 0.5 | 0.5 | 0.5 |
| Stearyltrimethylammonium | 4 | 4 | 4 | 4 | 4 | 4 | 4 |
| chloride | | | | | | | |
| Vaseline | 2 | 2 | 2 | 2 | 2 | 2 | 2 |
| Microcrystalline wax | | | | | | | |
| Phenoxyethanol | 0.2 | 0.2 | 0.2 | 0.2 | 0.2 | 0.2 | 0.2 |
| Hydroxyethanediphosphonic | 0.15 | 0.15 | 0.15 | 0.15 | 0.15 | 0.15 | 0.15 |
| acid | | | | | | | |
| Tetrasodium | 0.3 | 0.3 | 0.3 | 0.3 | 0.3 | 0.3 | 0.3 |
| hydroxyethanediphosphonate | | | | | | | |
| 35% hydrogen peroxide | 15.7 | 15.7 | 15.7 | 15.7 | 15.7 | 15.7 | 15.7 |
| Purified water | Proper amount | Proper amount | Proper amount | Proper amount | Proper amount | Proper amount | Proper amount |
| Total | 100 | 100 | 100 | 100 | 100 | 100 | 100 |
| Viscosity at 30 rpm (mPa · s) | 7640 | 7640 | 6840 | 7640 | 7640 | 7640 | 7640 |
| after 3 days | | | | | | | |
| Viscosity at 12 rpm (mPa · s) | 12650 | 12650 | 12750 | 12650 | 12650 | 12650 | 12650 |
| after 3 days | | | | | | | |
| V₃₀/V₁₂(s) | 0.60 | 0.60 | 0.54 | 0.60 | 0.60 | 0.60 | 0.60 |
| (V₃₀/V₁₂(f))/(V₃₀/V₁₂(s)) | 1.02 | 1.09 | 1.23 | 1.04 | 1.02 | 1.08 | 1.12 |
| (V₃₀/V₁₂(s))/(V₃₀/V₁₂(f)) | 0.98 | 0.92 | 0.82 | 0.96 | 0.98 | 0.92 | 0.90 |
| Equal amount discharge | ⊚ | ⊚ | ⊚ | ⊚ | ⊚ | ⊚ | ⊚ |
| properties | | | | | | | |

TABLE 17

| | Comparative | Comparative | Comparative | Comparative | Comparative | Comparative |
|---|---|---|---|---|---|---|
| | Example 1 | Example 2 | Example 3 | Example 4 | Example 5 | Example 6 |
| First agent | | | | | | |
| Behenyl alcohol | | | | | | |
| Arachyl alcohol | | | | | | |
| Stearyl alcohol | 0.5 | 3 | 3 | 3 | 3 | 3 |
| Cetanol | 2 | 4 | 4 | 4 | 4 | 4 |
| POE(30) cetyl ether | 2 | 2 | 2 | 2 | 2 | 2 |
| POE(2) cetyl ether | 1 | 1 | 1 | 1 | 1 | 1 |
| POE(2) stearyl ether | | | | | | |
| POE(50) oleyl ether | | | | | | |
| Stearyltrimethylammonium chloride | 1 | 1 | 1 | 1 | 1 | 1 |
| Sodium lauryl sulfate | | | | | | |
| Coconut oil fatty acid amidopropyl betaine | | | | | | |
| Vaseline | 3 | 8 | | 3 | 3 | 3 |
| Microcrystalline wax | | | 8 | | | |
| Liquid paraffin | | | | | | |
| Cetyl octanoate | | | | | | |
| Sodium sulfite | 0.1 | 0.1 | 0.1 | 0.1 | 0.1 | 0.1 |
| p-Phenylenediamine | 0.5 | 0.5 | 0.5 | 0.5 | 0.5 | 0.5 |
| m-Aminophenol | 0.4 | 0.4 | 0.4 | 0.4 | 0.4 | 0.4 |
| Resorcin | 0.5 | 0.5 | 0.5 | 0.5 | 0.5 | 0.5 |
| Ascorbic acid | 0.2 | 0.2 | 0.2 | 0.2 | 0.2 | 0.2 |
| 28% ammonia water | 4 | 4 | 4 | 4 | 4 | 4 |
| Purified water | Proper | Proper | Proper amount | Proper amount | Proper amount | Proper amount |
| | amount | amount | | | | |
| Total | 100 | 100 | 100 | 100 | 100 | 100 |
| Viscosity at 30 rpm (mPa · s) after 3 days | 3080 | 8020 | 11910 | 11130 | 11130 | 11130 |
| Viscosity at 12 rpm (mPa · s) after 3 days | 6570 | 21200 | 25200 | 18580 | 18580 | 18580 |
| V₃₀/V₁₂(f) | 0.47 | 0.38 | 0.47 | 0.60 | 0.60 | 0.60 |
| Amount of nonionic surfactant | 3 | 3 | 3 | 3 | 3 | 3 |
| Amount of oily component | 3 | 8 | 8 | 3 | 3 | 3 |
| C(n)/C(a) | 1.20 | 0.43 | 0.43 | 0.43 | 0.43 | 0.43 |
| C(i)/C(n) | 0.33 | 0.33 | 0.33 | 0.33 | 0.33 | 0.33 |
| Emulsion stability | Δ | ◯ | ◯ | ⊚ | ⊚ | ⊚ |
| Hair dyeing performance | ⊚ | ⊚ | ⊚ | ⊚ | ⊚ | ⊚ |
| Second agent | | | | | | |
| Stearyl alcohol | 1 | 1 | 1 | 1 | 1 | 1 |
| Cetanol | 4 | 4 | 4 | 4 | 4 | 4 |
| POE(30) cetyl ether | 1 | 1 | 1 | 3 | 1 | 1 |
| POE(2) cetyl ether | 0.5 | 0.5 | 0.5 | 1 | 0.5 | 0.5 |
| Stearyltrimethylammonium chloride | 4 | 4 | 4 | 1 | 1 | 1 |
| Vaseline | 2 | 2 | 2 | 2 | 12 | |
| Microcrystalline wax | | | | | | 5 |
| Phenoxyethanol | 0.2 | 0.2 | 0.2 | 0.2 | 0.2 | 0.2 |
| Hydroxyethanediphosphonic acid | 0.15 | 0.15 | 0.15 | 0.15 | 0.15 | 0.15 |
| Tetrasodium hydroxyethanediphosphonate | 0.3 | 0.3 | 0.3 | 0.3 | 0.3 | 0.3 |
| 35% hydrogen peroxide | 15.7 | 15.7 | 15.7 | 15.7 | 15.7 | 15.7 |
| Purified water | Proper | Proper | Proper amount | Proper amount | Proper amount | Proper amount |
| | amount | amount | | | | |
| Total | 100 | 100 | 100 | 100 | 100 | 100 |
| Viscosity at 30 rpm (mPa · s) after 3 days | 7640 | 7640 | 7640 | 5470 | 4310 | 4220 |
| Viscosity at 12 rpm (mPa · s) after 3 days | 12650 | 12650 | 12650 | 12810 | 8980 | 8780 |
| V₃₀/V₁₂(s) | 0.60 | 0.60 | 0.60 | 0.43 | 0.48 | 0.48 |
| (V₃₀/V₁₂(f))/(V₃₀/V₁₂(s)) | 0.78 | 0.63 | 0.78 | 1.40 | 1.25 | 1.25 |
| (V₃₀/V₁₂(s))/(V₃₀/V₁₂(f)) | 1.29 | 1.60 | 1.28 | 0.71 | 0.80 | 0.80 |
| Equal amount discharge properties | X | X | X | X | X | X |

### Industrial Applicability

According to the first embodiment of the present invention, an aerosol-type hair cosmetic material composition capable of lessening a residual amount in a pouch container is provided.

According to the second embodiment of the present invention, a second agent excellent in stability of hydrogen peroxide even under irradiation with sunlight is provided. That is, a second agent suitable for filling in a double structure container in which its residual amount is viewable is provided. In addition, an aerosol-type hair cosmetic material composition and an aerosol-type hair cosmetic material product, each of which is constituted to include above stated second agent, are provided.

According to the third embodiment of the present invention, a hair cosmetic material in which the generation of an oxygen gas to be caused due to contact between a first agent and a second agent, each of which has leaked into the inside of an outer container from a bag-like body in a double structure container, is reduced is provided.

According to the fourth embodiment of the present invention, a hair cosmetic material which is capable of performing equal amount discharge of a first agent and a second agent by a double structure container which is provided with a specified discharge mechanism, and has rheology properties with which equal amount discharge properties can be kept with time is provided.

1. -5. (canceled)

6. A hair cosmetic material composition for dispensing as an aerosol and, that is an oxidation hair dyeing agent or a hair bleaching agent, comprising a first agent containing an alkali agent and a second agent containing an oxidizing agent, wherein
the first agent and the second agent are contained in a double structure container in which a first inner container filled with the first agent and a second inner container filled with the second agent, each of which is independently provided, are accommodated in a same outer container, a space between the outer container and each of the inner containers is a propellant filling space filled with, a propellant, and the container is configured to simultaneously discharge the first agent and the second agent;
each of the first inner container and the second inner container is a respective pouch container formed by stick adhering together peripheries of sheet materials having a single-layer or multilayer structure including a resin layer, and an innermost layer of the pouch container is a polyolefin resin layer;
the agent filled in each of the pouch containers contains a nonionic surfactant and a higher alcohol and satisfies a condition of a content ratio of the nonionic surfactant to the higher alcohol of 0.13 to 0.6 on a mass basis; and
a content of higher alcohol having 14 to 18 carbon atoms to the entire higher alcohol content in each of the first agent and the second agent is 0.7 or more on a mass ratio basis.

7.-14. (canceled)
